(19) United States
(12) Patent Application Publication
(10) Pub. No.: US 2018/0371408 A1
(43) Pub. Date: Dec. 27, 2018

(54) CELL CULTURES AND METHODS OF USE

(71) Applicant: UNIVERSITY OF GEORGIA RESEARCH FOUNDATION, INC., Athens, GA (US)

(72) Inventor: Xiaozhong Yu, Watkinsville, GA (US)

(21) Appl. No.: 16/014,324

(22) Filed: Jun. 21, 2018

Related U.S. Application Data
(60) Provisional application No. 62/523,342, filed on Jun. 22, 2017.

(51) Int. Cl. C12N 5/076; C12N 5/00

(52) U.S. Cl. CPC C12N 5/061; C12N 5/0062; C12N 2533/50; C12N 2503/02; C12N 2320/10; C12N 2502/04; C12N 2510/04

(57) ABSTRACT

Provided herein is a composition that includes spermatogonial cells, Sertoli cells, and Leydig cells, and an optional protein matrix. In one embodiment, the cells are immortalized. In one embodiment, the cells in the composition are present at fractions that mimic a mouse testis at approximately 5 days postnatal. Also provided is a method for producing a cell culture that includes the cells of the composition, and methods of using the composition. In one embodiment, the composition can be used to detect compounds that alter the status of a cell in the composition, such as reduce viability of a cell.

### CROSS-REFERENCE TO RELATED APPLICATIONS

This application claims the benefit of U.S. Provisional Application Ser. No. 62/523,342, filed Jun. 22, 2017, which is incorporated by reference herein.

### GOVERNMENT FUNDING

This invention was made with government support under R21 OH 010473 awarded by the CDC, and R43 ES 027374 awarded by the National Institutes of Health. The government has certain rights in the invention. (37 CFR 401.14(f) (4))

### SUMMARY

The inventor has found that use of immortalized cell in a cell culture model described herein results in an unexpected increase in the reproducibility and sensitivity compared to the same cell culture model based on primary cells (Yu et al., 2005, Toxicol Sci, 84(2):378-93). Cytotoxicity (LC50) data generated from the co-culture system described herein are more consistent with the in vivo animal's reproductive toxicity as compared to a primary cell culture system (Yu et al., 2005, Toxicol Sci, 84(2):378-93). The use of immortalized cells significantly reduced source of variations in obtaining primary cells, and reduced the operation time, which significantly increases the power of sensitivity in detecting the potential toxicity of compounds.

Provided herein is a composition including cells and a protein matrix. The cells include spermatogonial cells, Sertoli cells, and Leydig cells. In one embodiment, the cells are immortalized. The spermatogonial cells can be present at 70-90%, the Sertoli cells can be present at 10-20%, and the Leydig cells can be present at 1-10%, where the spermatogonial cells, Sertoli cells, and Leydig cells add up to at least 100% of the cells in the composition.

The protein matrix includes a protein mixture that can represent an extracellular matrix. In one embodiment, extracellular matrix molecules can include collagen, fibronectin, laminin, vitronectin, tenascin, entactin, thrombospondin, elastin, gelatin, fibrillin, merosin, anchorin, chondronectin, link protein, bone sialoprotein, osteocalcin, osteopontin, epinectin, hyaluronectin, undulin, epiligrin, Kalinin, glycosaminoglycans, proteoglycans, a chemoattractant, a cytokine, a growth factor, or a combination thereof.

The spermatogonial cells, Sertoli cells, and Leydig cells can be rodent cells. An example of a spermatogonial cell is a C18-4 cell, and example of a Sertoli cell is a TM3 cell, and an example of a Leydig cell is a TM4 cell.

Also provided is a method for producing a cell culture. In one embodiment, the method includes combining cells and a protein matrix in a container to result in a cell culture, and incubating the cell culture under conditions suitable for maintaining viability of the cells. The cells include spermatogonial cells, Sertoli cells, and Leydig cells. In one embodiment, the cells are immortalized. In one embodiment, the spermatogonial cells are present at 70-90%, the Sertoli cells are present at 10-20%, the Leydig cells are present at 1-10%, and the spermatogonial cells, Sertoli cells, and Leydig cells add up to 100% of the total cells.

The protein matrix includes a protein mixture that can represent an extracellular matrix. In one embodiment, extracellular matrix molecules can include collagen, fibronectin, laminin, vitronectin, tenascin, entactin, thrombospondin, elastin, gelatin, fibrillin, merosin, anchorin, chondronectin, link protein, bone sialoprotein, osteocalcin, osteopontin, epinectin, hyaluronectin, undulin, epiligrin, Kalinin, glycosaminoglycans, proteoglycans, a chemoattractant, a cytokine, a growth factor, or a combination thereof.

Further provided are methods of using the composition. In one embodiment, the composition is present in a container, such as a well of a multi-well plate. The method includes contacting the cells with a compound to form a mixture, and incubating the mixture under conditions suitable for maintaining viability of the cells in the absence of the compound. The method also includes determining the status of cells.

In one embodiment, the status includes changes of cell cycle, DNA damage, nuclear shape, cell proliferation, and/or cytoskeleton. In one embodiment, the status includes cell viability. The method can further include determining whether the compound affects that status of the spermatogonial cells, the Sertoli cells, the Leydig cells, or a combination thereof. A compound can alter the status of cells, e.g., reduce cell viability, or have no detectable effect. In one embodiment, determining includes measuring neutral red uptake capacity of the cells.

In another embodiment, the method including contacting a composition described herein with a compound and analyzing viability of the cells, where a reduction of viability indicates the compound is a toxic compound.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The term “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

### BRIEF DESCRIPTION OF THE FIGURES

FIGS. 1A-E show a comparison between the testicular co-culture model (FIG. 1A and FIG. 1B) and single cell culture models, including spermatogonia (FIG. 1C), Leydig cells (FIG. 1D) and Sertoli cells (FIG. 1E). The testicular cell co-culture model consisted of three cell types: Spermatogonial cells (C18-4), Leydig cells (TM3), and Sertoli cells (TM4); the morphological images of co-culture are shown in FIG. 1A (48 h) and FIG. 1B (72 h). Single cell cultures of spermatogonial (C18-4, FIG. 1C), Leydig (TM3, FIG. 1D) and Sertoli cell culture (TM4, FIG. 1E) are shown at 48 h and 72 h. Cells were fixed with 4% paraformaldehyde, stained with antibodies, and automated multi-channel images were acquired using ArrayScan VTI with 40× magnification (ThermoScientific, MA). Multi-channel images show the morphology, including nuclear (Hoechst 33342, blue), cytoskeleton F-actin (phalloidin, green) and mitotic marker phosphor-histone H3 (pink). All experiments included three replicates, and were repeated three times. White arrows in FIG. 1A indicate mitotic cells, and white arrows in FIG. 1D and FIG. 1E indicate dividing cells with an F-actin “contract ring.” Scale bars: 50 μm.

FIGS. 2A-D show single-cell-based quantification of F-actin cytoskeletal structure. The multi-channel images were automatically captured using an Arrayscan VTI HCS reader with HCS Studio 2.0 Morphology Explorer BioApplication module (ThermoScientific, MA). Cytoskeletal rearrangement analysis was conducted in the images obtained from the co-culture model (FIG. 2A), and automatic segmentation of the nuclei images cells (blue line, FIG. 2B) and cellular outline (yellow line, FIG. 2C) were conducted. The localization and orientation of F-actin fibers were determined (FIG. 2C, green). A statistical summary of F-actin fibers was identified from bar charts and scatter plots (FIG. 2D). Actin fibers greater than a threshold length were identified and labeled with green, and the fiber intensity over 100 pixels were highlighted with red overlay (FIG. 2D), indicating F-actin bundle formation across cells in the co-culture model.

FIGS. 3A-D show single-cell-based high content analysis (HCA) of cell population (FIG. 3A), nuclear morphology (FIG. 3B) and F-actin cytoskeleton (FIG. 3C, FIG. 3D) in the testicular cell co-culture model and single spermatogonia culture model. The single-cell-based analysis of the cell populations in the co-culture as compared with the spermatogonial culture model is shown in FIG. 3A. The scatter plots of nuclear area versus nuclear total intensity at 48 or 72 h post-inoculation denote the cell sub-populations, as shown in the color-code chart. Quantifications of nuclear morphology changes, including nuclear number, area, and shapes (P2A and LWR) are shown in FIG. 3B. Quantifications of F-actin fiber distribution and variation of fiber intensity (the first-order texture) are shown in FIG. 3C, including the geometric mean of F-actin Fiber count (Spot Fiber Count), average and total intensity of fibers, variability of the intensity distribution (VarInten), arrangement and alignment of the fibers inside the cell (FiberAlign1 and FiberAlign2), geometric means of kurtosis (KurtIntenCh3), Skewness (SkewIntenCh3), and entropy (EntropyIntenCh3). Quantifications of F-actin spatial arrangements (the second-order texture) are shown in FIG. 3D. The second-order texture measures of F-actin reflected the spatial arrangements of the different pixels, including the maximum probability (MaxCoocIntenCh3), angular second moment (ASMCoocIntenCh3), entropy (EntropyCoocIntenCh3) and contrast (ContrastCoocIntenCh3). Data were presented as geometric mean of each well, and the linear line fit across the two-time-point was conducted.

Statistical analysis was conducted by one-way ANOVA followed by Tukey-Kramer multiple comparison (*p≤0.05 and **p≤0.001). The shaded area reflects the 95% confident intervals.

FIGS. 4A-F show cadmium treatment-induced destruction of F-actin and γH2AX expression in the testicular co-culture model. The representative images show the alteration of F-actin distribution and γH2AX expression levels in control (FIG. 4A), 0.05 (FIG. 4B), 0.25 (FIG. 4C), 0.50 (FIG. 4D), and 1.0 (FIG. 4E) μM. The in vitro co-culture was estabolised from the testicular cell lines—including Spermatogonial stem cells, Sertoli cells, and Leydig cells—with ECM overlay for 24 hours, and then treated with cadmium for 48 h. Multi-parametic analysis shows nuclear (Hoechst 33342, blue), cytoskeleton F-actin (phalloidin, green), and primary phospho-γH2AX followd with secondary Dylight 650 conjugated antibody (red). The images show a representative field from 49 fields captured for each well. High-content analysis of F-actin and γH2AX expression was conducted, and the percentage of the cell number, fluorescence intensity of f-actin, and γH2AX over the control were calculated (FIG. 4F). Data were presented as mean±SD, n=6. Three replicates in two separate experiments were included. Statistical analysis was conducted by 1-way ANOVA followed by Tukey-Kramer multiple comparison (*P<0.05, **P<0.01). Scale bars: 50 μm.

FIGS. 5A-B show immunofluorescence of the testicular cell co-culture model with cell-type-specific protein markers at 48 h (FIG. 5A) and 72 h (FIG. 5B) post-inoculation. Representative images show the three cell types of testicular cells in the co-culture model. Spermatogonial cells (C18-4) are labeled with a specific mouse germ cell nuclear antigen (GCNA1, pink staining in nucleus); Leydig cells are labeled with steroidogenic acute regulatory protein (StAR, red staining in cytoplasm, black arrow); and Sertoli cells are labeled with neither GCNA1 nor StAR (white arrow).

FIGS. 6A-D show comparison of cell viability of the tested compounds for 48 h exposure in the co-culture model. The in vitro co-culture model was estabolised from the testicular cell lines—including Spermatogonial stem cells, Sertoli cells, and Leydig cells—with ECM overlay for 24 hours, and then treated with 32 compounds for 48 h. Cell viability was assessed using a NR dye uptake assay. Data are presented as mean±SD of five replicates. The compounds were sorted into four groups based on the highest concentrations tested. Statistical analysis was conducted by one-way ANOVA followed by Tukey-Kramer multiple comparison. The compounds in FIG. 6A caused a statistically significant decrease in viability with all concentrations at 24 and 48 h (p<0.05). The compounds (DBP, DEHP, and BBP) in FIG. 6B caused a statistically significant decrease in viability at 200 μM (p<0.05). The compounds in FIG. 6C caused a statistically significant decrease in viability at 200 μM (p<0.05). No statistically significant changes were observed with the compounds in FIG. 6D at the concentrations below 1 mM, except HU. The data represent the mean of three independent experiments (four technical replicates for each experiment). Bars represent the standard deviation of the means.

FIG. 7 shows comparison of the relative toxicity of 32 tested compounds using a radar plot. The axis values were calculated as Log₂IC50 of individual compound using a culture model subtracted by the average of Log₂IC50 from all tested culture models. C18-4, TM3, and TM4 indicate spermatogonial, Leydig, and Sertoli cell cultures, respectively.

FIG. 8 shows hierarchical cluster analysis of ICs values of 32 tested compounds in the testicular co-culture model based on cell viability assay. Non-supervised two-dimensional hierarchical clustering analysis of IC20, IC50, and IC85 was conducted using the average linkage and elucidation dissimilarity method. IC20, IC50 and IC85 were Log10-transformed prior to analysis. Gradient color indicates the relative level of the log-transformed IC values. Since the IC values for the low toxicity chemicals could not be derived through calculation, the maximum dose of 5000 μM was selected. The toxicity rankings of different categories of chemicals were assessed.

FIG. 9 shows linear regression between in vivo reproductive toxicity (rLOAEL values) and IC50 values from the in vitro co-culture or single cell culture models at 48 h. Equations and R² are listed for each panel of regression plot. The co-culture at 48 h had the highest R² value (0.532).

FIG. 10 shows ML-based high-content and phenotypic analysis in the co-cultures. The diagram illustrates the four steps of the ML process. First, object identification and segmentation were conducted on multi-channel (nuclei, cytoskeleton, and γ-H2AX) images using CellProfiler. Second, over 200 quantitative features were extracted, including the size, shape, intensity, texture of the nuclei, intensity, and texture of F-actin, and intensity of γ-H2AX in a single cell. The orange-blue gradient represents the various features values (one per column) for a single cell. Then, a small training sample was developed by users with the manual classification of a specific phenotype in CellProfiler Analyst. Then, the ML algorithm was trained to discriminate between different phenotypes based on multiple features of the classified cells. Finally, the ML inferred classification rules from its training set to score all cells in the experiment and calculate the cell number in each class.

FIGS. 11A-C show cell viability determined by NR uptake assay in the co-cultures treated with BPA, BPS, BPAF, and TBBPA. Co-cultures were treated with various concentrations of BPA and BPS (25, 50, 100, 200 and 400 μM), and BPAF and TBBPA (2.5, 5, 10, 25 and 50 μM) for 24 (FIG. 11A), 48 (FIG. 11B), and 72 h (FIG. 11C). Cells treated with the vehicle (0.05% DMSO) were used as negative controls (0 μM). Data were expressed as mean±SD, n=8. Four replicates in two independent experiments were included. Statistical analysis was conducted by one-way ANOVA followed by Tukey-Kramer multiple comparisons (*P<0.05)

FIGS. 12A-G show characteristic changes of nuclear morphology and cell number in the co-cultures. Co-cultures were treated with various concentrations of BPA and BPS (5, 10, 25, 50 and 100 μM), and BPAF and TBBPA (1, 2.5, 5, 10, and 15 μM) for 24, 48, and 72 h. Cells treated with vehicle (0.05% DMSO) were used as negative controls (0 μM). The nuclei were stained with Hoechst 33342, and images were automatically acquired with 20× and 40× objective lenses, and 49 fields per well were obtained. FIG. 12A shows representative images (40×) of controls and cells treated with BPA and BPS (5, 10, and 100 μM), BPAF and TBBPA (5, 10, and 15 μM) for 48 h. Arrows indicate the multinucleated cells. Scale bar=50 μm. FIG. 12B shows the quantification of the absolute nuclear area (μm²), LWR for nuclear roundness, and P2A for nuclear smoothness. FIG. 12C-E show the quantification of cell number in each condition. FIG. 12F shows the representative images (20×) of multinucleated cell classification in control and the cells treated with BPA, BPS, BPAF, and TBBPA (5 μM) for 48 h, and quantification of multinucleated cell number using ML-based HCA. Using CellProfiler Analyst, multinucleated cells were automatically recognized and labeled with blue coloring. Non-multinucleated cells are labeled with orange coloring. Scale bar=100 μm. Data were presented as mean±SD, n=16. Four replicates in 4 independent experiments were included. Statistical analysis was conducted by one-way ANOVA followed by Tukey-Kramer multiple comparison (*P<0.05). FIG. 3G shows the Spearman correlation analysis of total F-actin intensity and total γ-H2AX intensity between non-multinucleated and multinucleated cells at a single cell level (*P<0.05). The shaded area indicates the 95% confidence interval. Arrows indicate the multinucleated cells with positive γ-H2AX foci staining in the nuclear and aberrant cytoskeleton distribution in the cytoplasm. Scale bar=50 μm.

FIGS. 13A-C show characteristic changes of the DNA synthesis and the cell population mitosis (M) phase in the co-culture. Co-cultures were treated with various concentrations of BPA and BPS (5, 10, 25, 50, and 100 μM), and BPAF and TBBPA (1, 2.5, 5, 10, and 15 μM) for 24, 48, and 72 h. Cells treated with vehicle (0.05% DMSO) were used as negative controls (0 μM). The nuclei were stained with Hoechst 33342 (blue). Cells were incubated with 5-bromo-2′-deoxyuridine (BrdU, 40 μM) for 3 h prior to cell fixation, and then stained with mouse anti-BrdU antibody and anti-mouse DyLight 488 for detection of BrdU incorporation (green). FIG. 13A shows the representative images (20×) of controls and the cells treated with BPA and BPS (100 μM), BPAF (5 and 15 μM), and TBBPA (15 μM) for 24 h. Arrows indicate the multinucleated cells with active DNA synthesis. Scale bar=100 μm. FIG. 13B shows the quantification of BrdU-positive cells. FIG. 13C shows the representative images (20×) of ML-based mitotic cell classification in control and cells treated with BPA and BPS (100 μM), and BPAF and TBBPA (15 μM) for 48 h; the image of nuclei in prometaphase, metaphase, anaphase, and late anaphase; and the quantification of cells in M phase. Using CellProfiler Analyst, cells in M phase were automatically recognized and labeled with blue coloring. Cells not in M phase are labeled with orange coloring. Scale bar=100 μm. Data were presented as mean±SD, n=8. Four replicates in two independent experiments were included. Statistical analysis was conducted by one-way ANOVA followed by Tukey-Kramer multiple comparisons (*P<0.05).

FIGS. 14A-E show characteristic changes of cytoskeleton and DNA damage response in the co-culture change of co-cultures treated with BPA, BPS, BPAF, and TBBPA. Co-cultures were treated with various concentrations of BPA and BPS (5, 10, 25, 50, and 100 μM), and BPAF and TBBPA (1, 2.5, 5, 10, and 15 μM) for 24, 48, and 72 h. Cells treated with vehicle (0.05% DMSO) were used as negative controls (0 μM). The nuclei were stained with Hoechst 33342 (blue), F-actin with Phalloidin staining (green), and γ-H2AX with a combination of primary anti-γ-H2AX and secondary Dylight 650 conjugated antibody (red). FIG. 14A shows the representative image (40×) of co-cultures treated with BPA and BPS (100 μM), BPAF (5 and 15 μM) and TBBPA (15 μM) for 48 h. Scale bar=50 μm. FIG. 14B demonstrates the quantification of log-transformed F-actin total intensity. FIG. 14C shows the representative images (20×) of ML-based classification of cells with stretching F-actin filaments in vehicle controls for 24, 48, and 72 h, and quantification of cells with stretching F-actin filaments in vehicle controls for 24, 48, and 72 h. Using CellProfiler Analyst, cells with stretching F-actin filaments were automatically recognized and labeled with blue coloring. Cells without stretching F-actin filaments are labeled with orange coloring. Scale bar=100 μm. Linear regression fit across multiple doses was performed. The shaded area indicates 95% confidence. FIG. 14D shows quantification of cells with stretching F-actin filaments in co-cultures. FIG. 14E shows the quantification of positive γ-H2AX cells. Data were presented as mean±SD, n=8. Four replicates in two independent experiments were included. Statistical analysis was conducted by one-way ANOVA followed by Tukey-Kramer multiple comparison (*P<0.05).

FIG. 15 shows HCA of cell cycle of co-cultures treated with BPA, BPS, BPAF and TBBPA. Co-cultures were treated with various concentrations of BPA and BPS (5, 10, 25, 50, and 100 μM), and BPAF and TBBPA (1, 2.5, 5, 10, and 15 μM) for 24, 48, and 72 h. Cells treated with vehicle (0.05% DMSO) were used as negative controls (0 μM). FIG. 15 shows the quantification of the percentage of each cell cycle stage, including sub-G1, G0/1, S, and G2/M phase. Data were presented as mean±SD, n=8. Four replicates in two independent experiments were included. Statistical analysis was conducted by one-way ANOVA followed by Tukey-Kramer multiple comparison (*P<0.05).

The following detailed description of illustrative embodiments of the present disclosure may be best understood when read in conjunction with the following drawings.

### DETAILED DESCRIPTION OF ILLUSTRATIVE EMBODIMENTS

Provided herein are compositions that include testicular cells and methods for making and using the compositions. A composition includes spermatogonial cells, Sertoli cells, and Leydig cells. The cells are in vitro (e.g., the cells are present in an artificial environment, such as a test tube or culture dish). The cells useful herein are cells that are capable of long term culture in tissue culture medium. A population of cells having a uniform genetic makeup, e.g., one type of cell in a culture, is also referred to herein as a cell line. Typically, a cell useful herein has the characteristic of being immortalized, and can be maintained in long term cultures.

Spermatogonial cells, also referred to as gonocytes, can have morphological features of type A spermatogonia, and express germ cell-specific genes such as GFRA1, Dazl and Ret, and stem cell specific genes such as piwi12 and prame11. In one embodiment, whether a cell is a spermatogonial cell can be determined using typical markers of mouse germ cell nuclear antigen (GCNA1). Spermatogonial cells can be obtained from any mammal. In one embodiment the cells are obtained from a rodent, such as a mouse or rat. Methods for obtaining spermatogonial cells include the STAPUT method that utilizes gravity sedimentation on a 2%-4% bovine serum albumin (BSA) gradient (Dym et al., 1995, Biol Reprod., 52:8-19; Hofmann et al., 2005, Stem Cells, 23(2):200-210). In one embodiment a spermatogonial cell is immortalized. As used herein an immortalized cell is one that does not undergo senescence and instead can continue to undergo division. Methods for immortalizing a cell, such as a spermatogonial, Sertoli, or Leydig cell, are known in the art and include, but are not limited to expression of certain viral genes. An example of a useful spermatogonial cell line is the C18-4 cell line, a cell line established by transfecting mouse spermatogonial stem cells with a plasmid allowing the expression of the SV40 large T antigen under the control of a ponasterone A-driven promoter (Hofmann et al., 2005, Stem Cells, 23(2):200-210).

Sertoli cells express follicle stimulating hormone, androgen receptor, and progesterone receptor. In one embodiment, whether a cell is a Sertoli cell can be determined by identifying the presence of SOX9, a Sertoli cell specific nuclear protein. For instance, anti-SOX9 antibody can be used to determine if SOX9 is present. In one embodiment the cells are obtained from a rodent, such as a mouse or rat. Methods for obtaining Sertoli cells are known (Chang et al., 2011, Biotechniques, 51(5): 341-344). In one embodiment a Sertoli cell is immortalized. Methods for immortalizing a Sertoli cell are known (Hofmann et al., 1992, Exp Cell Res. 201(2):417-35). An example of a useful Sertoli cell line is the TM4 cell line, available from the ATCC® as CRL-1715™.

Leydig cells express androgen receptor and progesterone. In one embodiment, whether a cell is a Leydig cell can be determined using steroidogenic acute regulatory protein (StAR) or identifying the presence of 3β-HSD, a Leydig cell specific protein. For instance, anti-3β-HSD antibody can be used to determine if 3β-HSD is present. In one embodiment the cells are obtained from a rodent, such as a mouse or rat. Methods for obtaining Leydig cells are known (Chang et al., 2011, Biotechniques, 51(5): 341-344). Methods for immortalizing a Leydig cell are known (Hofmann et al., 1992, Exp Cell Res. 201(2):417-35). An example of a useful Leydig cell line is the TM3 cell line, available from the ATCC® as CRL-1714™.

The spermatogonial cells in a composition make up from 70% to 90% of the cells in the composition (e.g., 70%, 71%, 72%, 73%, 74%, 75%, 76%, 77%, 78%, 79%, 80%, 81%, 82%, 83%, 84%, 85%, 86%, 87%, 88%, 89%, or 90%). The Sertoli cells in a composition make up from 10% to 20% of the cells in the composition (e.g., 10%, 11%, 12%, 13%, 14%, 15%, 16%, 17%, 18%, 19%, or 20%). The Leydig cells in a composition make up from 1% to 10% of the cells in the composition (e.g., 1%, 2%, 3%, 4%, 5%, 6%, 7%, 8%, 9%, or 10%). In one embodiment, the spermatogonial cells, Sertoli cells, and Leydig cells are present in the composition at fractions that mimic mouse testis at approximately 5 days postnatal, for instance 80%, 15%, and 5%, respectively (e.g., at a ratio of 16:3:1). In one embodiment, the spermatogonial cells, Sertoli cells, and Leydig cells make up essentially all the cells in the composition, thus the fractions of these three cell types add up to 100%. In one embodiment, the amount of each cell type in a composition can mimic the fractional percent present in the mouse testis at 5 days postnatal, e.g., 80% spermatogonial cells, 15% Sertoli cells, and 5% Leydig cells. In one embodiment, the composition can include one or more additional cells types. The one or more additional cells types can be present at, for instance, no more than 0.1% to 1% of the total number of cells.

A composition disclosed herein also includes a protein matrix. In one embodiment, a protein matrix represents an extracellular matrix. As used herein, the terms “extracellular matrix” and “ECM” are used interchangeably and refer to a natural or artificial composition that includes extracellular molecules typically secreted by cells and providing structural and/or biochemical support for cells. In some embodiments, a protein matrix includes proteins from solubilized basement membrane extracted from a tissue, such as testicular tissue. Examples of ECM molecules include, but are not limited to, collagen, fibronectin, laminin, vitronectin, tenascin, entactin, thrombospondin, elastin, gelatin, fibrillin, merosin, anchorin, chondronectin, link protein, bone sialoprotein, osteocalcin, osteopontin, epinectin, hyaluronectin, undulin, epiligrin, Kalinin, glycosaminoglycans, proteoglycans, chemoattractants, cytokines, growth factors, and a combination thereof (Ingber et al., US Published Patent Application 20170158997). One example of a useful protein matrix is a gelatinous protein mixture secreted by Engelbreth-Holm-Swarm (EHS) mouse sarcoma cells. This gelatinous protein mixture is available from Corning Life Sciences and BD Biosciences under the trade name MATRIGEL (Hughes et al., 2010, Proteomics, 10(9):1886-1890), and from Trevigen, Inc. under the trade name CULTREX BME.

The amount of protein matrix present in a composition can vary. In one embodiment, an amount of protein matrix used is sufficient to cover the surface of a container holding the composition. In another embodiment, an amount of protein matrix used is sufficient to produce a three-dimensional gel. Examples of concentrations of a protein matrix include, but are not limited to, at least 10 micrograms/ml (μg/ml), at least 50 μg/ml, or at least 75 μg/ml, and no greater than 200 μg/ml, no greater than 175 μg/ml, no greater than 150 μg/ml, or no greater than 125 μg/ml. In one embodiment, the amount of protein matrix used is at least 75 μg/ml to no greater than 125 μg/ml, such as 100 μg/ml.

In one embodiment, when present in a container, e.g., a well of a multi-well plate, the composition can include F-actin connecting cells in a three-dimensional structure. In one embodiment, after incubation for 48 hours a composition can include bundles of F-actin stretching out to other cells and forming cord-like structures.

### Methods of Making

Also provided are methods for making a composition described herein. A composition can be produced by combining cells and a protein matrix in a suitable container and incubating the cell culture under conditions suitable for maintaining the viability of the cells. Suitable conditions include, but are not limited to, 37° C., 5% CO₂, and a standard medium for culturing rodent cells. In one embodiment, the three types of cells (spermatogonial cells, Sertoli cells, and Leydig cells) are combined to result in a composition described herein and then added to a suitable container. The protein matrix described herein can be added to the container before, with, or after addition of the cells. Examples of a suitable container includes, but is not limited to, standard cell culture containers such as a multi-well plate, e.g., a 96-well plate, and other containers used in tissue culture. The total number of cells added to a container can be adjusted to result in 70% to 80% confluence after overnight incubation. For instance, 1.5×10⁴ cells can be used to inoculate a well of a 96-well plate. A cell culture can be used in a method described herein after overnight incubation, but other incubation times before use in a method are possible.

### Methods of Use

Further provided are methods for using a composition described herein. In one embodiment, a method includes providing a composition of cells described herein, and contacting cells in the composition with a compound, also referred to as a test compound, to form a mixture. The cells are typically present in a container, such as a well of a multi-well plate. At the time the compound is added the cells can be present in the container at a level of confluence of at least 50% (50 percent of the surface is covered by the cells), at least 60%, at least 70%, at least 80%, at least 90%, or at least 100% (the surface is completely covered by the cells) confluence. In one embodiment, the cells are at 70% to 80% confluence.

The compound can be one that is known to be or suspected of being a toxicant. As used herein, a “toxicant,” “testicular toxin,” and “reproductive toxicant” are used interchangeably and refer to a compound that can be toxic to a male reproductive system. A compound can be a chemical compound, including, for instance, an organic compound, an inorganic compound, a protein, or a metal. The compound can be a member of a library of many compounds, or the method can be used to specifically test one or more specific compounds or specific classes of compounds. Examples of libraries include, but are not limited to, libraries from the National Toxicology Program or the Environmental Protection Agency. In one embodiment, a compound is one that does not require metabolic activation.

In one embodiment, a compound is assessed at concentrations that are physiologically relevant to a mammalian system. Such concentrations can be determined by the skilled person for each specific compound examined. In one embodiment, the compound is assessed in concentrations routinely found, or expected to be found, in mammalian tissue or systems (e.g., from routine exposure). In another embodiment, the compound is assessed in concentrations routinely found in the environment. In one embodiment, the compound is assessed at a concentration of 1 nM to 100 mM. In one embodiment, the concentration of the compound is assessed at from 1 μM to 100 mM. In one embodiment, the compound is at a concentration of at least 1 nM, at least 10 nM, at least 100 nM, at least 1 μM, at least 5 μM, at least 10 μM, at least 20 μM, or at least 50 μM. In one embodiment, the compound is at a concentration of no greater than 80 μM, no greater than 100 μM, no greater than 200 μM, no greater than 500 μM, no greater than 750 μM, no greater than 0.1 mM, no greater than 0.5 mM, no greater than 1 mM, no greater than 5 mM, no greater than 10 mM, no greater than 20 mM, no greater than 50 mM, no greater than 75 mM, or no greater than 100 mM. A compound can be assessed at different concentrations, e.g., different doses. Higher and lower concentrations may also be useful, depending upon the specific compound being assessed. Higher concentrations can be used for compounds that have limited to poor solubility. In one embodiment, an initial dose-response curve can be established and used to adjust a dose range of a compound. The compound can be dissolved in a solvent, e.g., an aqueous or organic solvent, prior to use.

The method also includes incubating the mixture under conditions suitable for maintaining viability of the cells in the absence of the compound. Such conditions include the use of standard media and methods for maintaining cultured rodent cells, such as cultured mouse cells, including TM3, TM4, and C18-4 cells.

The status of cells in the mixture is determined a suitable period of time after the compound is added, such as 12 hours, 24 hours, 48 hours, or 72 hours after addition of the compound. In one embodiment, the viability of the cells is determined. Cell viability can be determined using essentially any method. In one embodiment, cell viability is determined by measuring neutral red uptake capacity of cells. Neutral red is retained inside the lysosomes of viable cells, while the dye is not retained by dead cells. Dye retention is proportional to the number of viable cells, and can be measured based on a neutral red absorbance value. Cells treated with solvent alone can be used as a control group with cell viability set as 100%. After incubation with the compound, e.g., 12 hours, 24 hours, 48 hours, or 72 hours, the medium can be replaced with a medium containing neutral red at an appropriate concentration. Following incubation with the dye, cells can be washed, neutral red eluted with a suitable solution, such as a combination of acetic acid and ethanol, and absorbance values measured at 540 nm. Cell viability can be expressed as a percentage of the mean of solvent controls after subtracting a background control. In one embodiment, a compound is considered to be a toxicant for cells of the cell culture when there is a statistically significant reduction of cell viability compared to the solvent control. In one embodiment, a compound is considered to be a toxicant for cells of the cell culture when the fold change over the control is at least 0.1, at least 0.2, at least 0.3, at least 0.4, at least 0.5, at least 0.6, at least 0.7, at least 0.8, or at least 0.9. Optionally, the inhibitory concentration (IC) of a compound can be determined, including the IC₂₀, IC₅₀ (half maximal inhibitory concentration), and/or IC₈₅.

In another embodiment, the cell status evaluated can be changes of cell cycle, DNA damage, nuclear shape, cell proliferation, and/or cytoskeleton. Cytoskeleton can include F-actin cytoskeletal structure, such as intensity and distribution of actin fibers. Methods for evaluating these and other variables include, but are not limited to, single-cell based high-content analysis (Liang et al., 2017, Toxicol Sci, 155(1), 43-60).

A compound can affect one cell type in a composition, two cell types in a composition, or all cell types in a composition. Thus, in one embodiment, the method further includes determining whether the compound affects spermatogonial cells, Sertoli cells, Leydig cells, or a combination thereof.

A compound that alters the status of one or more cell types in the composition, e..g, reduces viability, is considered a possible toxicant, and optionally can be further tested using other model systems available to further evaluate whether the compound is a male reproductive toxicant. It is also expected that in some embodiments compounds will be identified that do not affect cell viability.

### Exemplary Embodiments

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### An Animal-Free in Vitro Three-Dimensional Testicular Cell Co-Culture Model for Evaluating Male Reproductive Toxicants

### Abstract

Primary testicular cell co-culture model has been used to evaluate testicular abnormalities during development, and was able to identify the testicular toxicity of phthalates. However, the primary testicular cell co-culture model has disadvantages in employing animals for the isolation of testicular cells, and the complicated isolation procedure leads to inconsistent results. We developed an in vitro testicular co-culture model from rodent testicular cell lines, including spermatogonial cells, Sertoli cells, and Leydig cells with specified cell density and Extracellular

Matrix (ECM) composition. Using comparative high-content analysis of F-actin cytoskeletal structure between the co-culture and single cell culture models, we demonstrated a three-dimensional structure of the co-culture, which created an in vivo-like niche, and maintained and supported germ cells within a three-dimensional environment. We validated this model by discriminating between reproductive toxicants and non-toxicants among 32 compounds in comparison to the single cell culture models. Furthermore, we conducted a comparison between the in vitro (IC50) and in vivo reproductive toxicity testing (lowest observed adverse effect level on reproductive system, rLOAEL) We found the in vitro co-culture model could classify the tested compounds into four clusters, and identify the most toxic reproductive substances, with high concordance, sensitivity, and specificity of 84%, 86.21%, and 100%, respectively. We observed a strong correlation of IC50 between the in vitro co-culture model and the in vivo testing results. Our results suggest that this novel in vitro co-culture model may be useful for screening testicular toxicants and prioritize chemicals for further assessment in the future.

### Introduction

Reproductive and developmental disorders caused by exposure to environmental chemicals are a prominent health issue worldwide (Jenardhanan et al., 2016; Leung et al., 2016). Animal testing for evaluating potential reproductive toxicity is one of the most complicated, time-consuming and expensive processes when examining complex endpoints. Testing under the current guidelines requires a large number of animals, ranging from 560-6,000 animals per chemical or drug (OECD, 2016). The implementation of the new European Registration, Evaluation, Authorization and Restriction of Chemical (REACH) program requires toxicological information to be submitted for about 30,000 existing chemicals. Although REACH promotes limiting vertebrate animal testing as far as possible, the lack of suitable alternatives will probably increase the use of animals (Hareng et al., 2005; Hartung and Rovida, 2009; Hofstetter et al., 2013; Luijten et al., 2007; Parks Saldutti et al., 2013; Sauer, 2004; Scialli and Guikema, 2012). Moreover, every year, approximately 700 new chemicals are introduced into the market, which imposes a great burden on reproductive and developmental toxicity testing. The Interagency Coordinating Committee on the Validation of Alternative Methods (ICCVAM) developed recommendations for minimum procedural standards and testing methods for the validation of in vitro estrogen receptor (ER) and androgen receptor (AR) binding and transcriptional activation (TA) assays (Casey, 2016; ICCVAM, 2012). So far, there are no validated alternative tests that would cover different aspects of the reproductive cycle. Thus, it has become increasingly important to develop an in vitro test that can serve as an equally effective alternative to animal testing for reproductive toxicity. In 2007, the U.S. Environmental Protection Agency (EPA) launched a large-scale program, ToxCast, to investigate high-throughput, in vitro assays to prioritize substances for further in-depth toxicological evaluation, identify mechanisms of action, and develop predictive models for in vivo biological response (Houck et al., 2009). The focus of the ToxCast program was to generate an in vitro bioactivity profile for each chemical, and correlate this profile with the toxicity data from in vivo animal studies (Auerbach et al., 2016; Karmaus et al., 2016; Kavlock et al., 2012; Paul Friedman et al., 2016). There are more than 500 assays across nine assay technology platforms, including cell-free high throughput screening assays and cell-based assays in multiple human and rodent primary and derived cell lines. Although a small number of assays were found to be associated with the identified reproductive toxicants, and showed a certain level of predictive power for reproductive toxicity (Leung, et al., 2016; Martin et al., 2011; Reif et al., 2010), there is no in vitro model in the ToxCast program designed specifically for detecting reproductive toxicity.

Currently, in vitro reproductive screening models for testicular development and spermatogenesis are actively being developed (Hareng, et al., 2005; Hofmann et al., 2005a; Luijten, et al., 2007; Parks Saldutti, et al., 2013; Yu et al., 2008; Yu et al., 2009; Yu et al., 2005). In vitro culture systems have been used to evaluate testicular changes during normal development (Bilinska, 1989; Chapin et al., 1988; Gray, 1986; Griswold, 1998; Hadley et al., 1985; Lejeune et al., 1998). Sertoli/gonocytes co-cultures (SGC) are used to examine cell-cell interactions, as well as effects of hormones and growth factors on spermatogonia survival and proliferation in vitro (Mather et al., 1990; Orth and Jester, 1995). Human fetal testis xenografted into the renal subcapsular space was developed to study the effects of toxicants on human tissues (Spade et al., 2014a; Spade et al., 2014b). Production of post-meiotic spermatids in explained pieces of testis were observed in both mouse and rat models (Brannen et al, 2016; Sato et al., 2011). These advances provide evidence that Sertoli and Leydig cells, as well as peritubular myoid or endothelial cells, are all essential in supporting and maintaining spermatogenesis in the testis. Self-renewal and progeny production of spermatogonia are controlled by the neighboring differentiated cells and extracellular matrix (ECM), known as substrate in vivo niches, while Sertoli cells are required for successful differentiation of germ cells in vitro culture systems (Griswold, 1998). The ECM Matrigel-based primary testicular cell model was reported to form a testicular-like multilayered architecture that mimics in vivo characteristics of seminiferous tubules (Harris et al., 2015; Wegner et al., 2013; Wegner et al., 2014; Yu, et al., 2008; Yu, et al., 2009; Yu, et al., 2005). As previously demonstrated, this model differentially responds to various phthalate exposures with changes in several cellular pathways, showing sensitive response to testicular toxicity (Harris, et al., 2015; Yu, et al., 2009). However, this in vitro primary testicular cell co-culture model has the disadvantage of employing animals for the isolation of testicular cells, and the complicated isolation procedure leads to inconsistent results (Wegner, et al., 2013). Therefore, in this study, we developed an in vitro testicular cell co-culture model from rodent testicular cell lines using spermatogonial cells (C18-4), Sertoli cells (TM4), and Leydig cells (TM3). We tested this animal-free in vitro testicular co-culture model with 32 compounds and compared their cytotoxicities with any single cell culture of spermatogonia, Sertoli cell or Leydig cells, and further conducted a comparison between the in vitro (IC50 of cell viability) and in vivo reproductive toxicity testing (lowest observed adverse effect level on the reproductive system). We observed that the in vitro co-culture model could classify the tested compounds into four clusters, and identified the most toxic reproductive substances, which had high concordance, sensitivity, and specificity values of 84%, 86.21%, and 100%, respectively. We observed a strong correlation of IC50 between this in vitro testicular co-culture model and the in vivo testing results. We have demonstrated that this novel in vitro co-culture model may be useful in screening testicular toxicants in a wide concentration range, and will help prioritize chemicals for future assessment.

### Materials and Methods

### Chemicals and Reagents

Dulbecco's modified Eagle's medium (DMEM), antibiotics (penicillin and streptomycin), fetal bovine serum (FBS), 0.25% trypsin/EDTA, and ethanol were purchased from GE Healthcare Life Sciences (Logan, Utah.). Nu-Serum culture supplement (Nu-serum) and Extracellular Matrix (ECM) Matrigel were from BD BioScience (Redford, Mass.). Glacial acetic acid was obtained from Merck (Darmstadt, Germany).

Both recognized reproductive toxicants and non-reproductive toxic compounds were selected for testing, as listed in Table 1. We selected 32 compounds, and obtained their in vivo toxicities by manually searching the literature and public sources, such as the LOAEL values provided in the ToxCast database (Chapin and Stedman, 2009; CIRM, 2008; Moorman et al., 2000). Most of compounds were purchased from Sigma-Aldrich (St. Louis, Mo.), Fisher Scientific (Gaithersburg, Md.), Chem Service (West Chester, Penn.) and GFS Chemicals (Powell, Ohio.), as indicated. Some compounds were graciously provided by the National Toxicology Program (NTP).

TABLE 1
List of Compounds Tested

| | | | | | | | In vivo* |
|---|---|---|---|---|---|---|---|
| | | | Molecular | Purity | | | Reproductive |
| Chemical Name | Abbreviation | CAS # | Weight | (%) | Application | Provider | Toxicity |
| Sodium arsenite | As | 7784-46-5 | 129.91 | 90 | Pesticides | GFS chemicals | + |
| Boric acid | BA | 10043-35-3 | 61.83 | 99.5 | Insecticide | Sigma | + |
| Benzyl butyl phthalate | BBP | 85-68-7 | 312.36 | 98 | Plasticizer | Sigma | + |
| Benzophenone-3,3′,4,4′- | BEN | 2421-28-5 | 322.23 | 96 | Solvent | Sigma | + |
| tetracarboxylic dianhydride | | | | | | | |
| 2,2-Bis(4-hydroxy-3- | BPA | 80-05-7 | 256.34 | 99 | Plasticizer | Sigma | + |
| methylphenyl)propane | | | | | | | |
| 4,4′- | BPAF | 1478-61-1 | 336.23 | 97 | Fire-retd | Sigma | NA |
| (Hexafluoroisopropylidene)diphenol | | | | | plasticizer | | |
| 4,4′-Sulfonyldiphenol | BPS | 80-09-1 | 250.27 | 98 | Flame | Sigma | NA |
| | | | | | retardant | | |
| Cadmium chloride | Cd | 10108-64-2 | 183.32 | 99.99 | Dye | Sigma | + |
| chlorpyrifos | CHL | 2921-88-2 | 350.59 | 97 | Pesticides | Chemservice | + |
| Cyclophosphamide | CYC | NA | 279.1 | 95 | Cancer | TCI | + |
| Dibutyl phthalate | DBP | 84-74-2 | 278.34 | 99 | Solvent | Sigma | + |
| Dioctyl phthalate | DEHP | 117-81-7 | 390.56 | 99.5 | Plasticizer | Sigma | + |
| Diethylphthalate | DEP | 84-66-2 | 222.24 | 99 | Plasticizer | Sigma | − |
| Diethylstilbestrol | DES | 56-53-1 | 268.35 | 99 | Nonsteroidal | Fisher | + |
| | | | | | estrogen | | |
| Valproic acid sodium salt | VPA | 1069-66-5 | 166.19 | 98 | Anticonvulsant | Sigma | + |
| Zearalenone | ZEA | 17924-92-4 | 318.36 | 99 | Pesticides | Sigma | + |

*In vivo toxicity of these compounds were based on ToxCast database as well as literature search. “+”, “−” and “NA” indicates the confirmed animal reproductive toxicants, non-reproductive toxicants or no data available, respectively.

### Cell Culture and Treatment

Mouse Leydig cells (TM3) and Sertoli cells (TM4) were purchased from ATCC. These cells were isolated from pre-pubertal mouse gonads (Mather, 1980; Mather and Phillips, 1984). TM3 cells specifically express androgen receptor and progesterone. TM4 cells specifically express follicle stimulating hormone, androgen receptor and progesterone receptor (Mather, 1980; Mather and Phillips, 1984). The mouse spermatogonial cell line C18-4 was established from germ cells isolated from the testes of 6-day-old Balb/c mice. This cell line shows morphological features of type A spermatogonia, and expresses germ cell-specific genes such as GFRA1, Dazl and Ret, and stem cell specific genes such as piwi12 and prame11. It proved to be an ideal cell model for studying the early phase of spermatogenesis, although the functional transplantations were not conducted to prove the stem cell nature (Hofmann, et al., 2005a; Hofmann et al., 2005b). The spermatogonial cells were maintained in DMEM medium composed of 5% FBS, and 100 U/ml streptomycin and penicillin in a 33° C., 5% CO2 humidified environment in a sub-confluent condition with passaging every 3-4 days. Leydig cells and Sertoli cells were cultured in DME/F12 medium supplemented with 100 U/ml streptomycin and penicillin, 5% horse serum, and 2.5% FBS, and maintained at 37° C. with 5% CO₂. For both the single cell culture models and testicular co-culture model, the cells were inoculated with 1.5×10⁴ per well in a 96-well plate, cultured overnight at 70-80% confluence, and treated with various concentrations of the testing compounds. For the testicular cell co-culture model, the cellular composition of fractions mimicked mouse testis around 5 days postnatal, and was 80%, 15% and 5% for gonocyte, Sertoli cell, and Leydig cell, respectively. Spermatogonial cells, Sertoli cells, and Leydig cells were mixed in this defined proportion and seeded into a 96-well plate in a DMEM/high glucose medium at 33° C., supplemented with 5% Nu-serum. ECM Matrigel (Corning, N.Y.) was added to each well for a final concentration of 100 μg/ml, and the plates were gently swirled to ensure dispersal of Matrigel after addition. Cells were cultured overnight and treated with various concentrations of testing compounds in the doses and time periods indicated.

### Assessment of Cell Morphology

All cultures were viewed with an Olympus inverted microscope equipped with phase-contrast optics (Olympus, Tokyo, Japan) at intervals during the culture to assess their general appearance. Images were captured and digitized with a Nikon Camera. To further examine the morphology of the cultured cells, a multi-parametric high-content analysis (HCA) was applied to quantify the F-actin cytoskeleton, nuclear shape, and cell proliferation. The spermatogonial cells were identified using typical markers of mouse germ cell nuclear antigen (GCNA1) (Developmental Studies Hybridoma Bank, http://dshb.biology.uiowa.edu, Iowa, IA)(Enders and May, 1994). Leydig cells were identified using steroidogenic acute regulatory protein (StAR)(Clark et al., 1994) (kindly gifted by Dr. Douglas M. Stocco, Texas Tech University). Sertoli cells were stained with SOX9 (Developmental Studies Hybridoma Bank, Iowa, IA). After culture for 48 and 72 h, cells were washed with phosphate buffered saline (PBS), fixed with 4% formaldehyde for 20 min at room temperature, and then washed with PBS three times. After permeabilization in Triton/PBS solution containing 0.1% Triton X-100 (TX-100) for 15 min, cells were blocked for 30 min in 3% bovine serum albumin (BSA, Sigma) in PBS with 0.1% TX-100, and then incubated with primary phospho-histone H3 antibody (1:200, Thermo Scientific) or phospho-γH2AX (γH2AX, Millipore-Sigma) in PBS and 0.1% TX-100 over night at 4° C. After washing with PBS/Tween-20 twice, cells were incubated with goat anti-mouse Dylight 650, goat anti-rabbit Dylight 550 (Thermo Scientific, MA), and Hoechst 33342 (Molecular Probes, OR) in PBS/BSA solution for 90 min at room temperature. Prior to image acquisition, cells were stained for 30 min at room temperature with Alexa Fluor 488 Phalloidin (Cell Signaling, MA) for F-actin staining. The multichannel images were automatically captured using an Arrayscan VTI HCS reader with a Studio 2.0 Target Activation BioApplication module (Thermo Scientific, MA). Thirty-six fields per well were acquired at 40× magnification using a Hamamatsu ROCA-ER digital camera in combination with 0.63× coupler and Carl Zeiss microscope optics for automatic image acquisition. Channel 1 (Ch1) applied the BGRFR 386_23 for Hoechst 33342, which was used for an auto-focus channel, and the objects (nuclei) were identified. For F-actin and γH2AX or Phospho-Histone H3 staining, Ch2 applied the BGRFR 485_20 for F-actin and Ch3 applied the BGRFR 650_13 for γH2AX or Phospho-Histone H3.

A four-channel assay was conducted to assess the cell-type specific marker staining for GCNA1 and StAR. Channel 1 was applied the BGRFR 386_23 for Hoechst 33342, Ch2 applied the BGRFR 485_20 for F-actin, Ch3 applied the BGRFR 650_13 for GCNA1, and Ch4 for StAR. For negative control, the primary antibody was omitted, and was stained with the secondary antibody only, indicating that the primary antibodies used were due to primary antibody specificity and not to unspecific binding of the secondary antibody to the cells.

Single-cell based High-content Analysis (HCA) provided multi-parameter phenotypic profiling characterization, including number, nuclear area, shape, and intensity, as previously reported (Liang et al., 2017). Nuclear shape measurement included P2A and LWR parameters. P2A, which evaluates nucleus smoothness, is a shape measurement based on the ratio of the nuclear perimeter squared to 4π*nucleus area (perimeter²/4π*nucleus area). LWR (length-width ratio), which evaluates nucleus roundness, measures the ratio of the length to the width of the nucleus. Total intensity was defined as the total pixel's intensities within a cell in the respective channel; the average intensity was defined as the total pixels' intensities divided by the area of a cell in the respective channel. With 36 fields of each well, at least 4000 cells were analyzed per well, and single-cell based data for each channel were exported for further statistical analysis. The experiments were performed with at least four biological replicates and repeated at least twice.

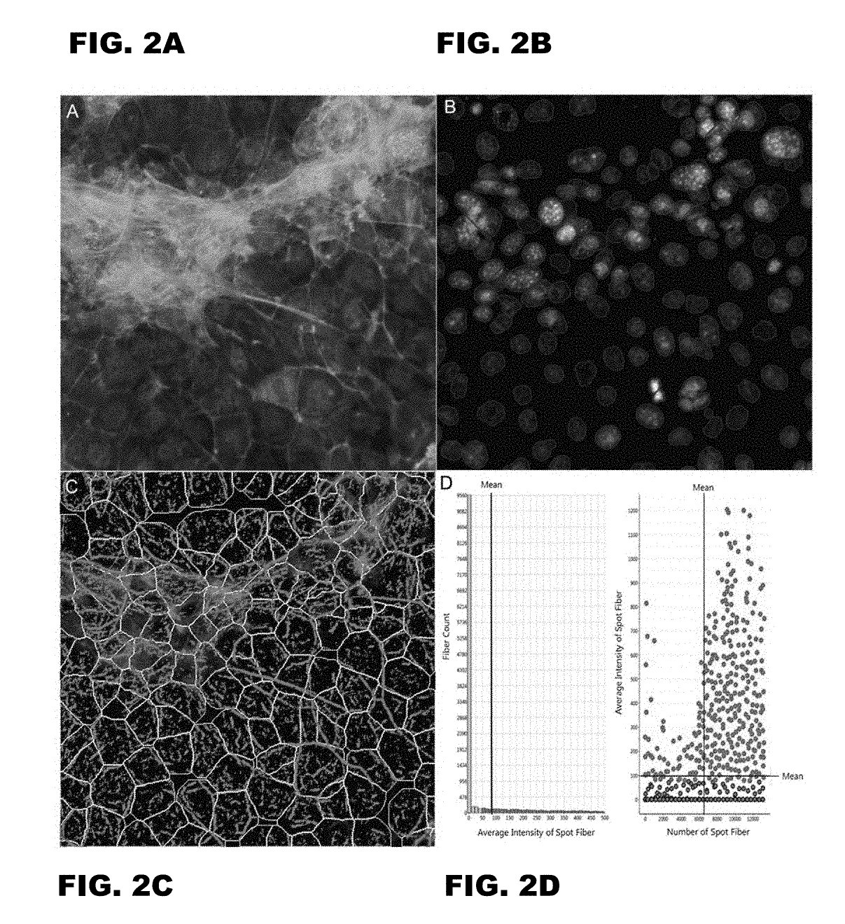

For quantitative analysis of three-dimensional F-actin cytoskeleton, we applied the Cytoskeletal Re-arrangement Assay in the Morphology Explorer Bioapplication (ThermoScientific, MA) to determine the number, dimensions and alignment—as well as the texture—of actin fibers based on the manufacturer's guideline (ThermoFisher, 2010). Briefly, for identification of fibers, pixels with high intensities were selected, and the threshold level controlled by the Assay Parameter Detection was set at a value of 1 (FIG. 2). There are two levels of texture measurements of F-actin. The first-order texture measures are based on the pixel intensity distribution, including mean (AvgIntentCh3) or total intensity of an object (TotalIntenCh3), variability of the intensity distribution (VarIntenCh3), skewness (SkewIntenCh3), kurtosis (KurtIntenCh3), entropy (EntropyIntenCh3), and the variation of surface area density (DiffIntenDensityCh3). FiberAlign1 reflects the arrangement and alignment of the fibers inside the cell. FiberAlign2 reflects the angle of each individual fiber's orientation with the axis of the image. The second-order texture measures are intensity-independent spatial arrangements of the different pixels. The co-occurrence matrix is calculated based on the number of occurrences of a pixel with a certain intensity being adjacent to a pixel of another specific intensity. Four parameters were reported, including maximum probability (MaxCoocIntenCh3), contrast (ContrastCoocIntenCh3), entropy (EntropyCoocIntenCh3), and angular second moment ASMCoocIntenCh3.

### Neutral Red (NR) Dye Uptake Assay

Cell viability was determined by measuring the capacity of cells to take up NR (Repetto et al., 2008b; Yu, et al., 2009; Yu, et al., 2005). NR is retained inside the lysosomes of viable cells, while the dye cannot be retained if the cells die. Dye retention is proportional to the number of viable cells, and can be measured based on NR absorbance value. Cells were seeded to 96-well plates, and treated with different compounds at four doses with five replicates. Cells treated with the vehicle (0.05% DMSO) were used as the background group with cell viability set as 100%. After 24 h or 48 h, the medium was replaced with a medium containing NR dye (50 μg/ml, 200 μl per well). Following 3 h incubation, the supernatants were removed, the cells were washed with PBS twice, and NR was eluted with 100 μl of a 0.5% acetic acid/50% ethanol solution. The plate was gently rocked on a plate shaker, and absorbance values were measured at 540 nm with a Synergy HT microplate reader (BioTek, VT). Cell viability was expressed as a percentage of the mean of vehicle controls after subtracting the background control. The initial testing concentrations of these compounds were determined based on the published cytotoxicity data. After examination of the initial dose-response curve from the co-culture model, the dose ranges were adjusted. For those compounds with less cytotoxicity, the highest concentrations tested were 50 mM and 5 mM in the water-soluble or DMSO vehicle, respectively.

### In Vivo Reproductive Toxicity Data and Comparison

The U.S. EPA's ToxCast program reviewed the in vivo animal studies and established the Toxicity Reference Database (ToxRefDB) (available on the workd wide web at actor.epa.gov/actor/home.xhtml). Reproductive lowest observed adverse effect levels (rLOAEL) from in vivo studies were generated, and reflected reproductive toxicities (Martin, et al., 2011). The endpoints for determining rLOAEL of in vivo studies include, but are not limited to, primary fertility, early offspring survival, offspring weight, longer-term offspring survival, and other systemic toxicities of offspring. As previously reported, in vivo reproductive toxicants were defined as having achieved an rLOAEL lower than 500 mg/kg/day (Martin, et al., 2011). Compounds such as BPS, BPAF, and TBBPA with insufficient in vivo reproductive toxicity data were marked as “NA” for “no available information.” A concordance analysis was performed to assess the degree of agreement of chemical positive or negative response between the NR assay and in vivo evaluation, or the percentage of cell viability from the NR assay that matched the calls from those in the literature. In addition, sensitivity (%) was calculated based on the formula: 100×(the proportion of chemicals with a positive result in an NR assay that were positive based on the literature calls). Similarly, specificity (%) was derived from the formula: 100×(the proportion of chemicals with a negative result in the NR assay that were negative based on the literature).

### Statistical Analysis

All data obtained from the HCS Studio™2.0 BioApplication were exported, and further analysis was conducted using the JMP statistical analysis package (SAS Institute, NC). The parameters from the single-cell based imaging were quantified, and the geometric mean for each well was determined. Data were presented as geometric mean±standard deviation (SD). Statistical significance was determined using one-way ANOVA followed by a Tukey-Kramer all pairs comparison. The cell viabilities were calculated as the arithmetic mean percentages of treated versus the respective control. The data represented the average±standard deviation of five replicates. IC20, IC50, and IC85 values were calculated using the logistic 4P models in the Sigmoid Curves fit in the JMP. Non-supervised Hierarchical Clustering analyses of dose-response curves (IC20, IC50 and IC85) of tested compounds were used to determine the binary category based on the average linkage and elucidation distance correlation coefficients using MeV software (Chittenden et al., 2012). Correlation of IC50 values against the published in vivo reproductive LOAEL data was calculated. The degree of correlation was examined based on the r value and the regression coefficient (R²). The Pearson correlation coefficient between IC50 values and rLOAEL were also calculated. Statistical analysis was conducted using JMP software (SAS Inc. Cary, N.C.).

### Results

### Construction of Co-Culture Model Using Testicular Cell Lines and Overview of Morphology

### Morphological Comparisons of the in Vitro Culture Models

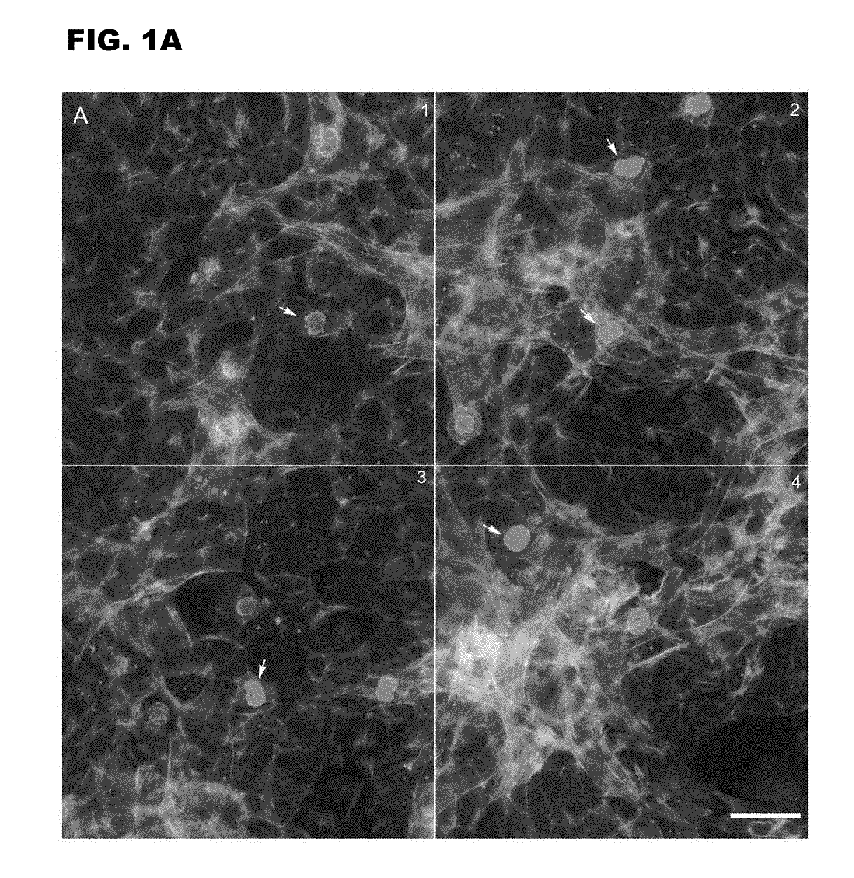

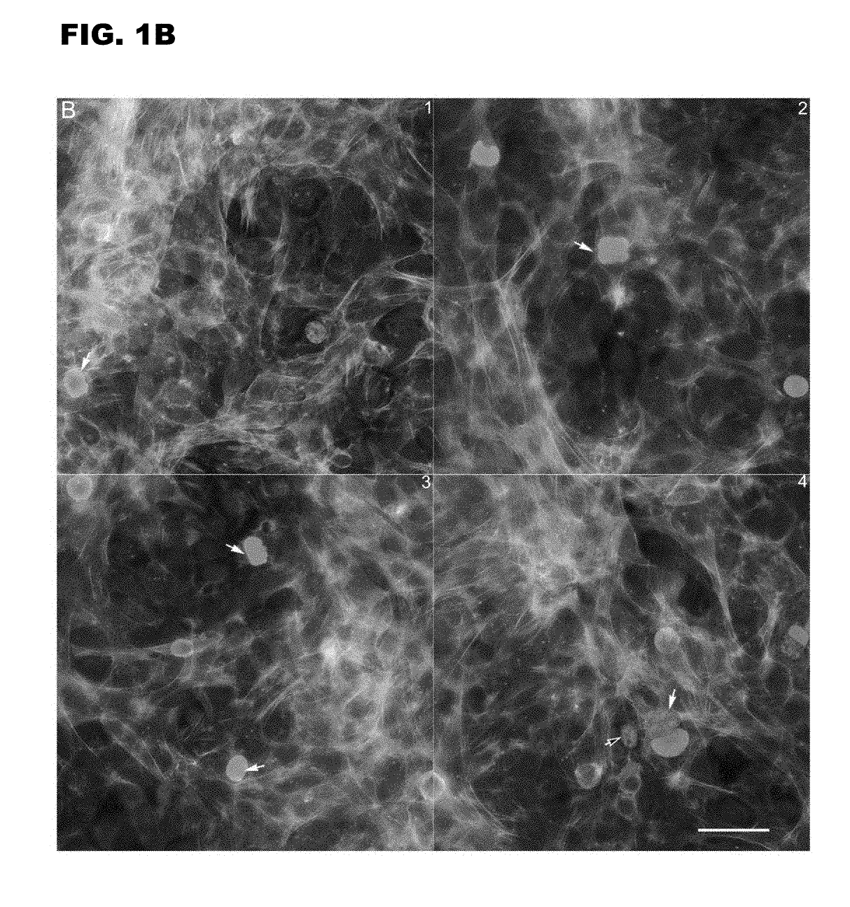

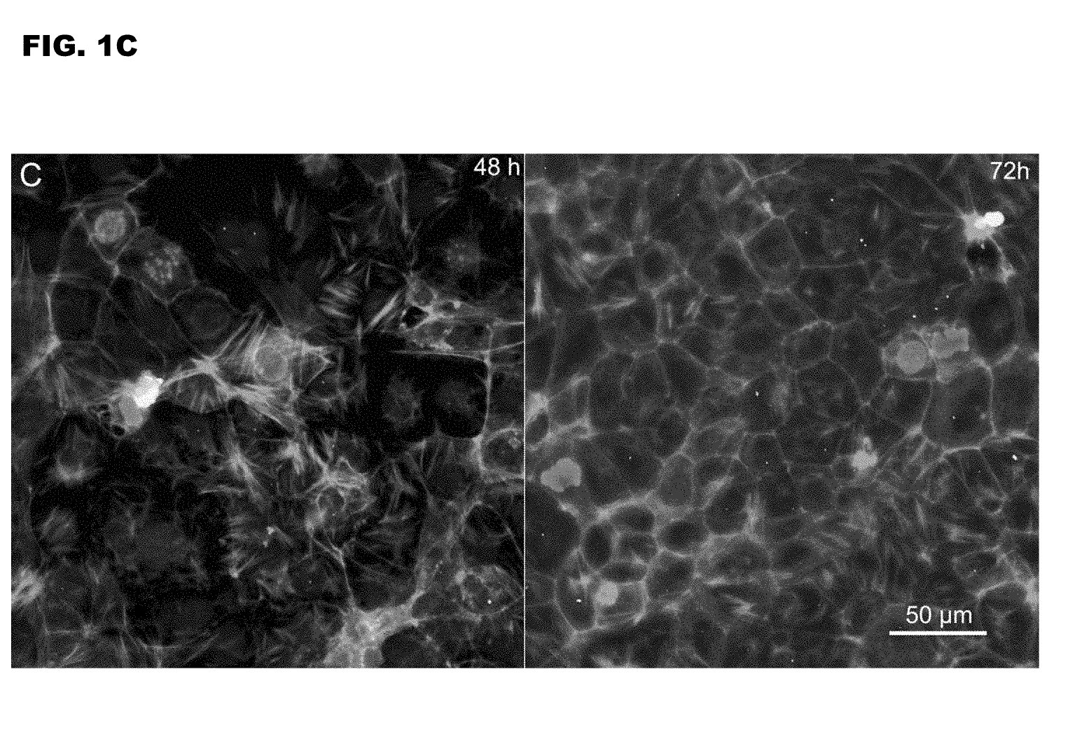

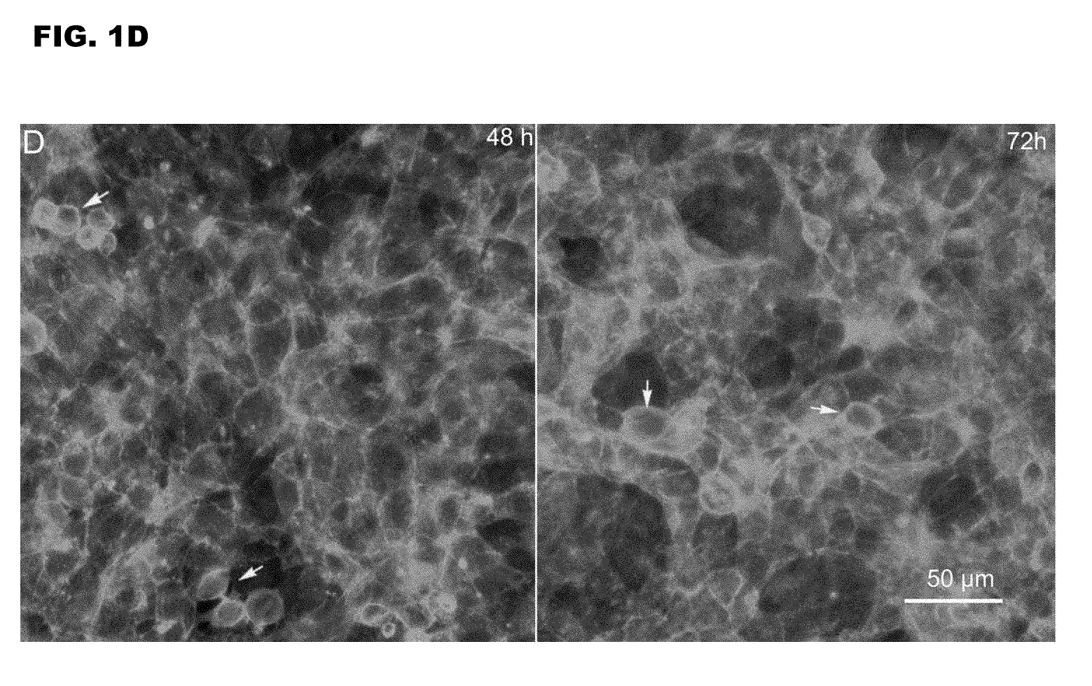

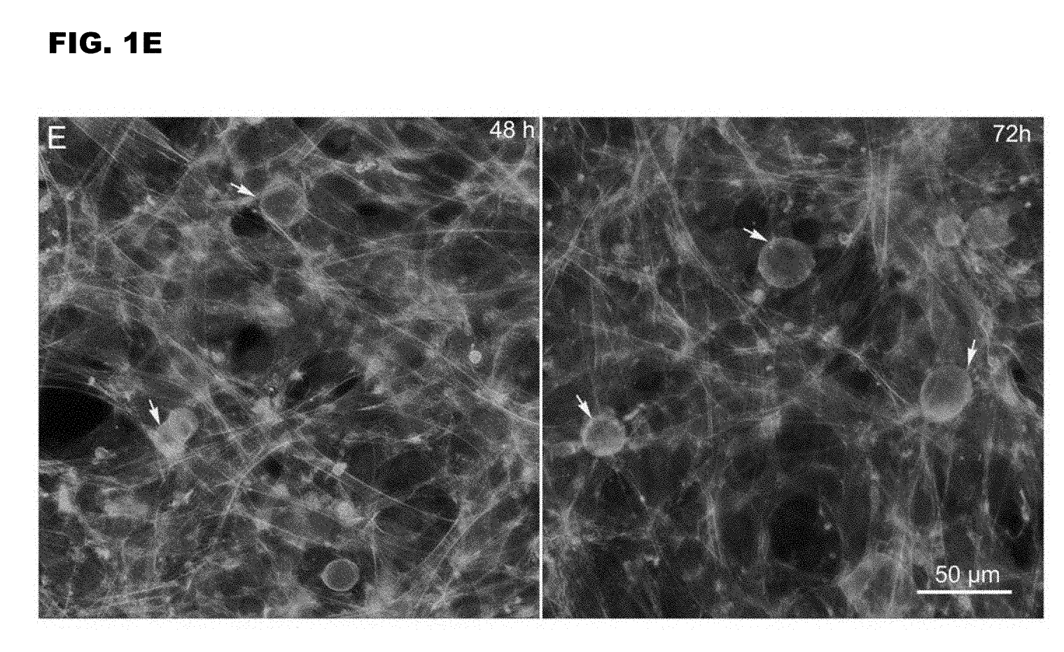

We established a testicular co-culture model using spermatogonial, Sertoli, and Leydig cells, based on the cellular composition of 5-day postnatal mouse testis. The testicular cells in a defined proportion were seeded in a 96-well plate in DMEM supplemented with 5% Nu-serum with an overlay of ECM. In order to examine whether the co-cultured cells grew into biomimetic and three-dimensional structures, we examined F-actin cytoskeleton along with cell proliferation markers (FIG. 1). As illustrated in FIG. 1A (48 h) and B (72 h), representative four-field images per well were shown to reflect the overall structure of nuclear (Hoechst 33322, blue), F-actin cytoskeleton (phalloidin, green), and mitotic status of cells (phosphorylated Histone H3, red). Overall, distinct F-actin cytoskeletal structures, including intensity and distribution of actin fibers, were observed in the co-culture model (FIG. 1A and 1B), as compared with the single cell culture models, including spermatogonial cells (FIG. 1C), Sertoli cells (FIG. 1D) and Leydig cells (FIG. 1E). We found unique cord-like multiple-layer structures with actively dividing cells in the co-culture (FIG. 1A and B), and observed mesh-like assembly and thicker bundles of F-actin filaments (green) across multiple cells in the co-culture model at 72 h post inoculation. These cellular structures observed in the co-culture model were noticeably different from any type of single cell cultures (FIG. 1C-E). As shown in Figure IC, the spermatogonial cells expressed strikingly dense cortex F-actin, which was localized at the cell boundary, but not notably in the cytoplasm. No obvious differences in the mitotic counts by phosphorylated Histone H3 staining were found between the co-culture and single spermatogonial cell culture models (white arrows). Like the spermatogonial cells, Leydig cells formed diffuse F-actin fibers without a distinct boundary between cells (FIG. 1D). Cells undergoing cell division with re-organization of cytoskeletal structure showed a distinct F-actin “contract ring” (FIG. 1D, white arrows). In contrast to the spermatogonia and Leydig cells, the fine F-actin fibers in the Sertoli cells stretched through the cytosol and interconnected across multiple cells. We also observed these “contract rings” in the dividing Sertoli cells (FIG. 1E, white arrows).

### Quantitative Analysis and Comparisons of Cytoskeleton

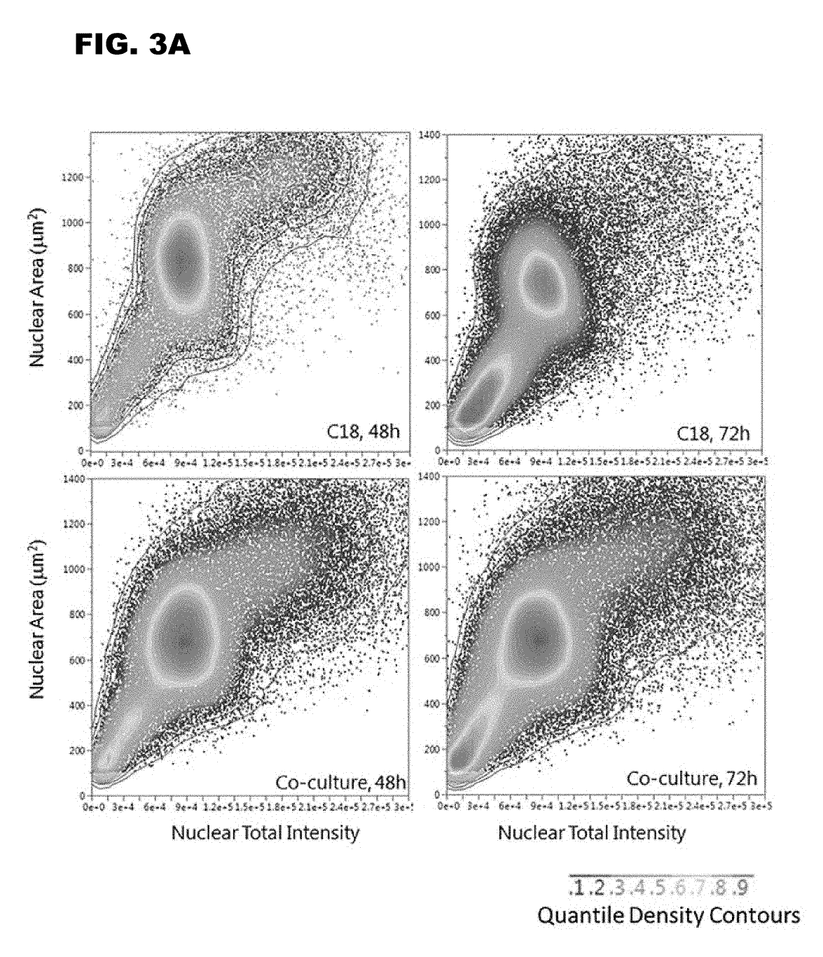

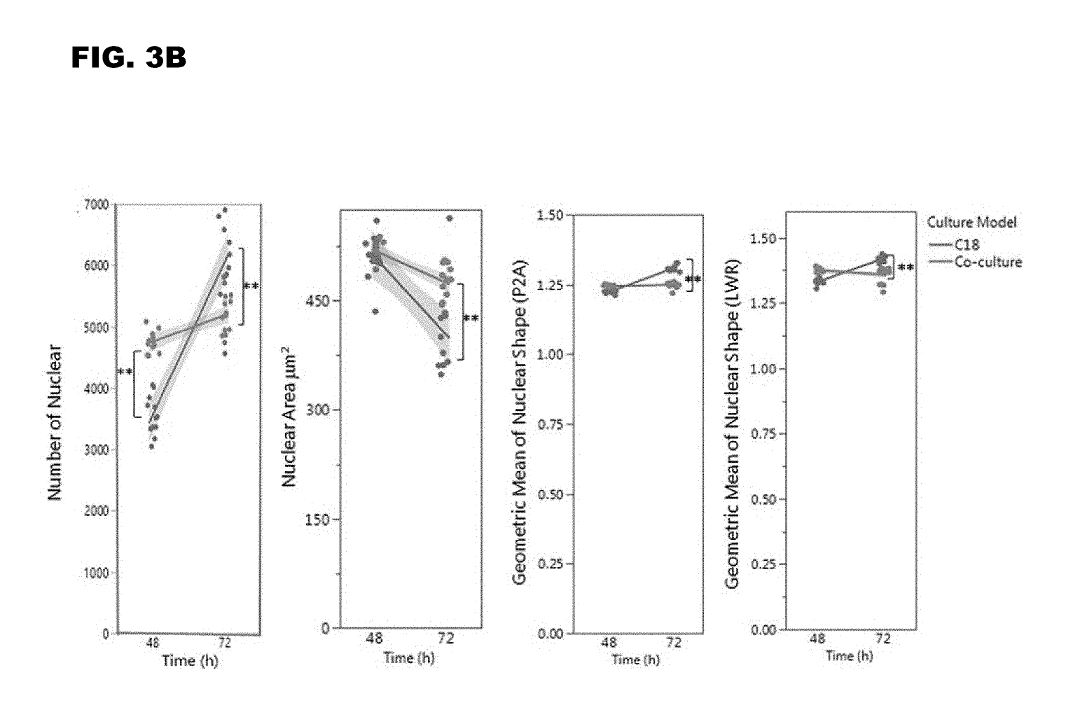

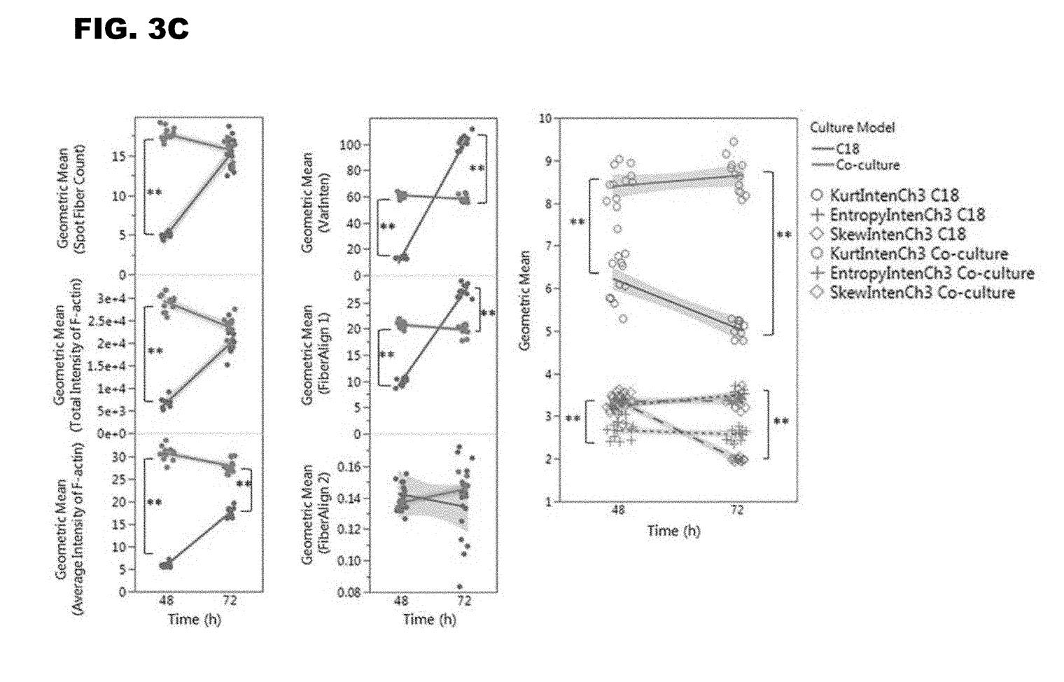

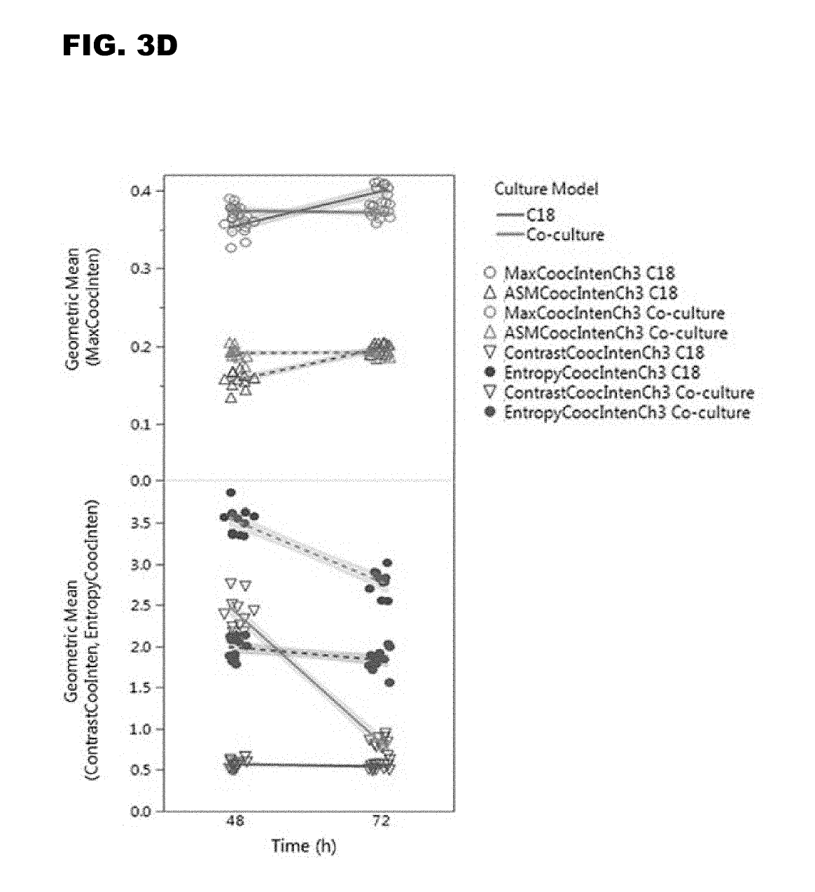

We further quantitatively characterized F-actin cytoskeleton using a Cytoskeletal Re-arrangement algorithm to demonstrate the three-dimensional structure in the co-culture. FIG. 2 illustrates the quantification of F-actin based on the images obtained from the co-culture model. Automatic segments of the nuclear (B, blue outline) and cell outlines (C, yellow outline) were conducted, and the location and orientation of F-actin fibers were determined. Actin fibers greater than a threshold length (1.6) were identified (D, green) and the fiber intensity over 50 pixels were highlighted with red. The overlay of red and green indicates that the actin filaments are organized into higher order structures, forming actin bundles across the cells in the co-culture model. FIG. 3A shows the scatterplot of nuclear area and total intensity of the nuclear staining, reflecting the distribution of cell populations in the co-culture as compared with the single cell culture condition. In contrast to the spermatogonia) cell culture, we observed an increase in the population with a smaller nuclear size in the co-culture, reflecting the existence of Sertoli and Leydig cells. Quantification of nuclear morphology (FIG. 3B) showed a larger number of cells in the co-culture at 48 h, with a slight increase at 72 h. In the single cell culture, the number of cells was lower as compared to the co-culture at 48 h, and increased significantly at 72 h. The average nuclear area was similar at 48 h between the two types of cultures, and decreased significantly at 72 h, especially for the single culture (FIG. 3B). There was no difference in nuclear shapes at 48 h, but significantly higher ratios of P2A (nucleus smoothness) and LWR were observed at 72 h in the single cell culture as compared to the co-culture (FIG. 3B). As shown in FIG. 3C, the distribution and arrangement of F-actin fibers were quantified (first-order texture measures), showing the spatio-temporal changes of F-actin in the co-culture model and single spermatogonia culture at 48 h and 72 h. The geometric mean of F-actin fiber count (SpotFiberCountCh3), both the average and total intensity of fibers (AvgIntenCh3/TotalIntenCh3), were higher in the co-culture as compared to a single cell culture at 48h, and maintained a similar level at 72 h (FIG. 3D). Significant increases in fiber counts (SpotFiberCountCh3), along with average and total intensity of fibers (AvgIntenCh3/TotalIntenCh3), were observed at 72 h after seeding, and reached at a similar level in the co-culture model. We observed higher variability of the intensity distribution (VarIntenCh3), arrangement, and alignment of the fibers inside the cell (FiberAlign1Ch3) in the co-culture than those in the single cell culture at 48 h; however, these parameters increased significantly in the single cell culture at 72 h, while maintaining similar levels in the co-culture (FIG. 3C). There were no significant differences in the measure of FiberAlign2Ch3 between the two types of cultures at both time-points. Regardless of the time-points, higher geometric means of kurtosis (KurtIntenCh3) were observed in the co-culture as compared to the single cell culture. The geometric mean of Skewness (SkewIntenCh3) was similar between the two types of culture at 48 h, and significantly decreased in the single cell culture at 72 h. The geometric mean of entropy (EntropyIntenCh3) was lower in the co-culture at both 48 and 72 h time-points. The second-order texture measures of F-actin reflecting the spatial arrangements of the different pixels are shown in FIG. 3D. Slightly lower maximum probability (MaxCoocIntenCh3) and angular second moment (ASMCoocIntenCh3) were observed in the single cell culture at 48 h, but MaxCoocIntenCh3 slightly increased at 72 h. As compared to the single cell culture, the co-culture model had lower levels of entropy (EntropyCoocIntenCh3) and contrast (ContrastCoocIntenCh3) at both time-points. Higher levels of entropy (EntropyCoocIntenCh3) and contrast (ContrastCoocIntenCh3) were observed at 48 h in the single cell culture, and then decreased at 72 h. All together, these parametric measurements of F-actin fibers showed distinct three-dimensional features of cytoskeleton in the co-culture. These higher order F-actin structures may enhance the cell-cell interactions of Sertoli, Leydig, and germ cells in the co-culture model, and improve the overall in vitro biological functionality.

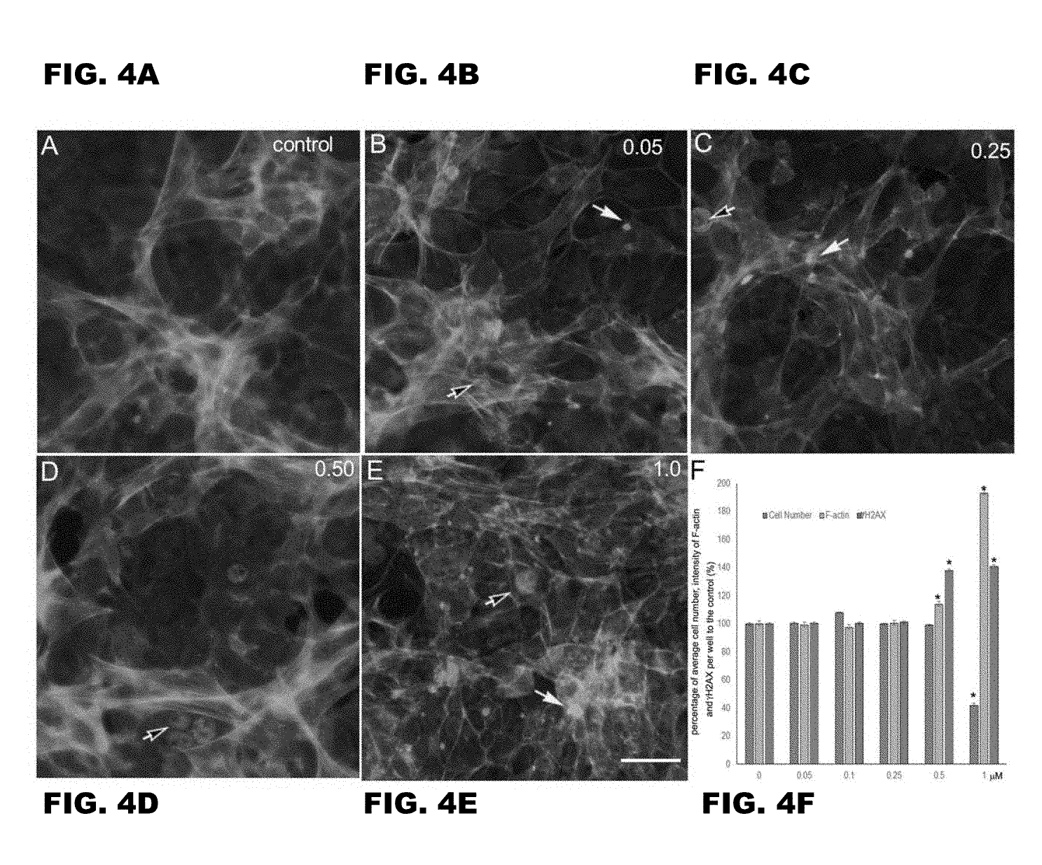

To assess the validity of toxicity testing using this co-culture model, we applied it to examine the effect of cadmium, a known reproductive toxicant, using a multi-parametric high-content assay. FIG. 4 illustrates the morphological changes of the co-culture with cadmium treatment at 0.05, 0.25, 0.50 and 1.0 μM for 48 h. We examined changes in nuclear morphology, cytoskeleton, and early DNA damage markers using γH2AX staining. Cadmium treatment induced significant morphological disruptions, including destruction of cytoskeletal structure and decrease of cell proliferation (cell number). Following exposure to cadmium, actin fibers were found to be truncated and depolymerized (annotated by white arrows). F-actin fibers were retrieved into the cell cytosol instead of stretching out across the cytosol as shown in the control cells. Further, cadmium significantly induced phosphorylation of γH2AX foci formation (black arrows) in a dose-dependent response (FIG. 4D).

### Characterization of Multiple Cell Types in the Co-Culture

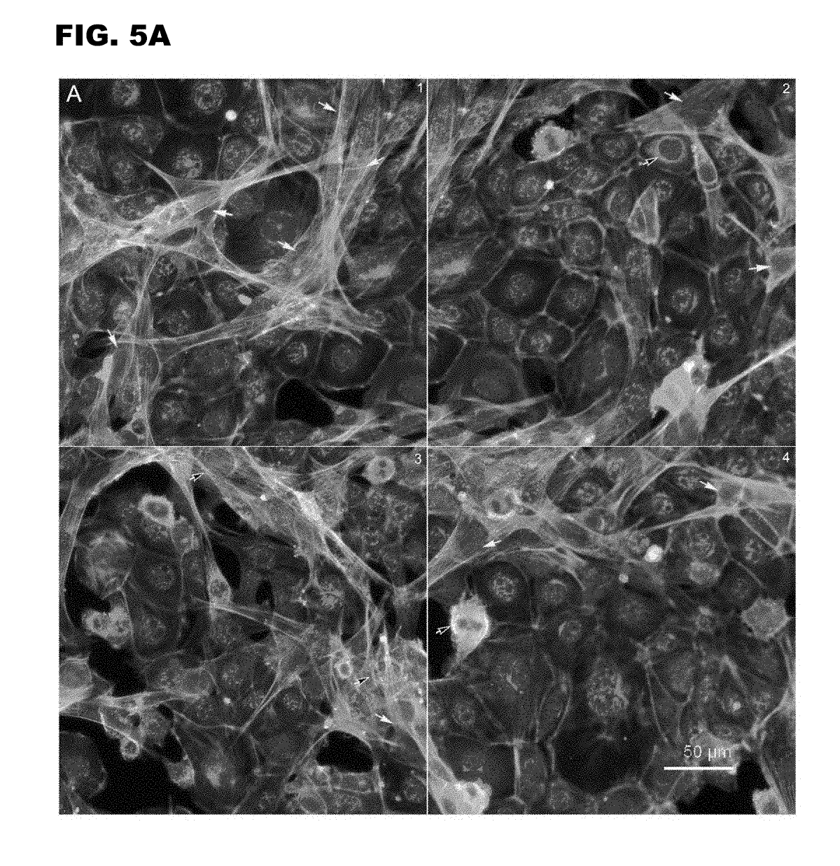

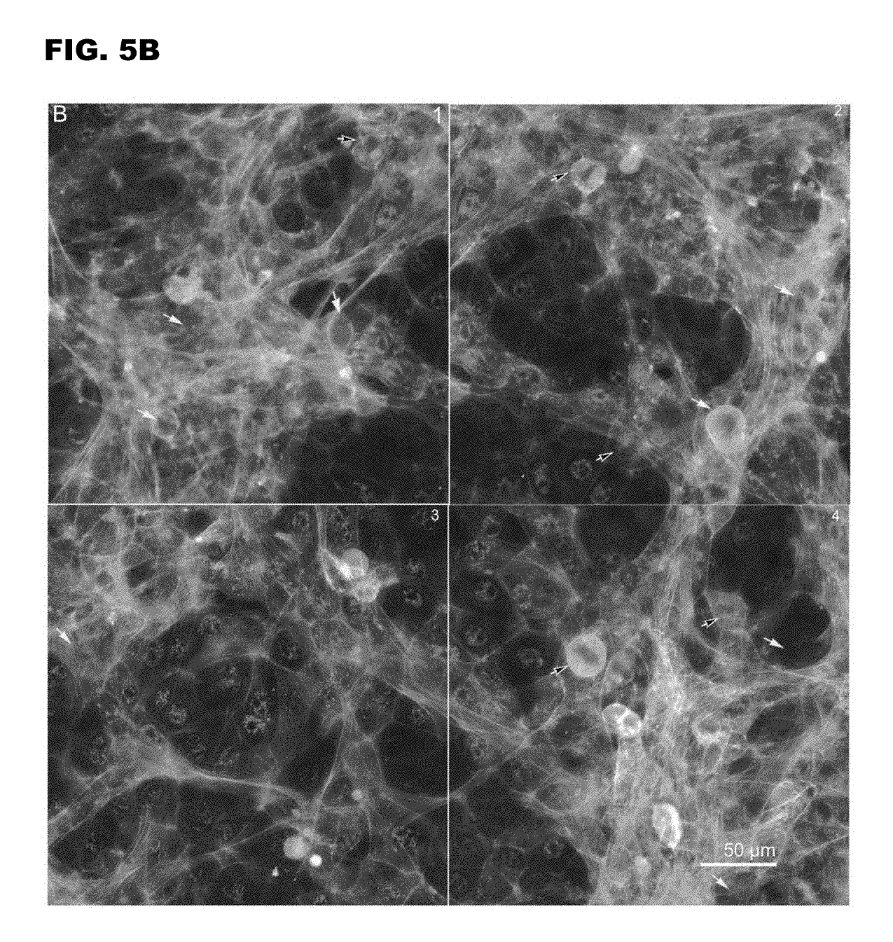

To further identify the composition of the micro-niches of the co-culture model, we applied cell-type specific protein markers to characterize the testicular cells in the co-culture. Germ cell nuclear antigen 1 (GCNA1) is a continually expressed protein specific to spermatogonial cells. Leydig cells were identified as having an antibody against steroidogenic acute regulatory protein (StAR). SOX9 is proposed to be a specific marker for Sertoli cells, but we found it also expressed in C18-4 and TM3 cells. As shown in FIG. 5, the cells with GCNAlnuclear staining (pink) were spermatogonial cell. The cells with blue nucleus and reddish StAR staining in the cytoplasm are Leydig cells (black arrows). The cells expressing neither GCNA1 nor StAR staining are indicated as Sertoli cells (white arrows). In general, these cells had thicker bundles of F-actin stretching out to other cells, and formed cord-like structures at 48 h (FIG. 5A). At 72 h (FIG. 5B) post inoculation, fine F-actin fibers formed a three-dimensional cytoskeletal network throughout the cells. The spermatogonial cells with nuclear GCNA1 pink staining were located within these cord-like structures.

### Comparison of Cell Viability of the Testicular Co-Culture Model by 32 Chemicals

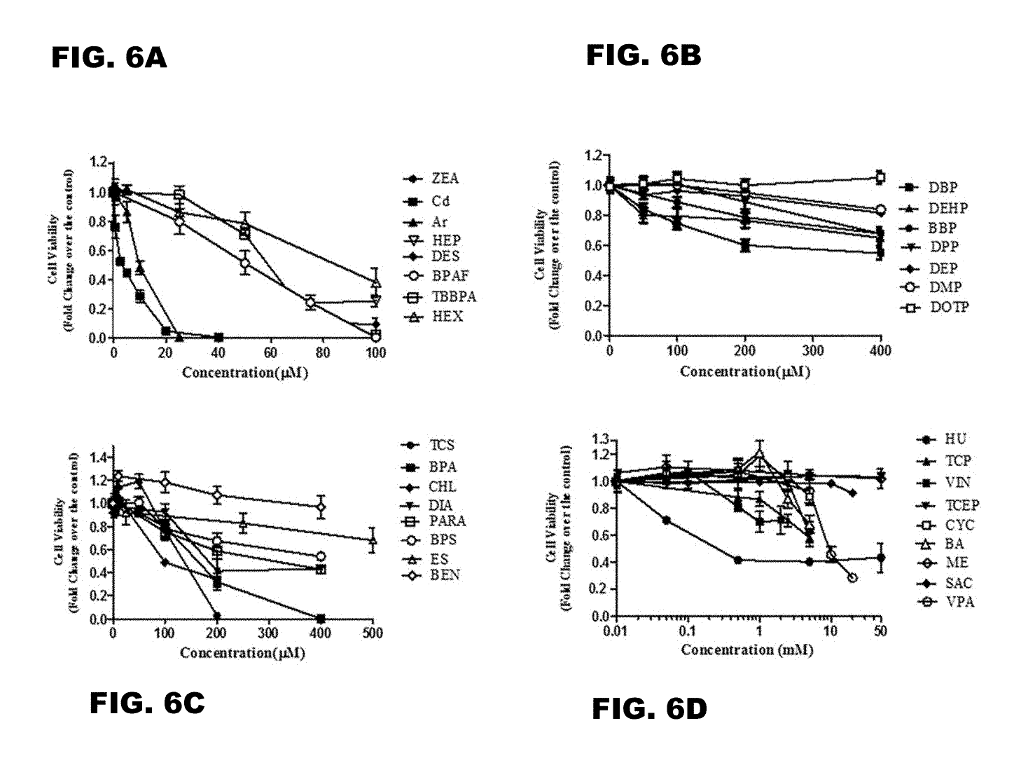

As the first step to validate whether this in vitro co-culture model can be used to screen testicular toxicants, we compared the cytotoxicity with known reproductive toxicants (Table 1). We applied the NR uptake assay to examine dose-dependent responses with those selected compounds in the co-culture model, as well as a single cell culture model for 24 and 48 h, including spermatogonial cells (C18), Sertoli cells, and Leydig cells. FIG. 6 shows dose-dependent cytotoxicity in response to 32 testing compounds in the co-culture model after 48 h treatment. These compounds were organized into four charts based on their highest concentrations of testing chemicals (FIG. 6A, B, C, and D). FIG. 6A includes compounds whose concentrations tested highest at 100 μM, including ZEA, Cd, As, HEP, DES, BPAF, TBBPA, HEX, and ZEA. Arsenic (As) and cadmium (Cd) were most toxic, followed by HEP, DES, BPAF, TBBPA and HEX. FIG. 6B shows the dose-dependent cytotoxicity of phthalate esters. Male developmentally toxic phthalate esters, including DBP, DEHP, BBP, and DPP, induced a dose-dependent decrease of cell viability after treatment for 48 h, while non-toxic phthalate esters, including DEP, DMP, and DOTP, induced no or slight decrease of cell viability. FIG. 6C shows a group of compounds, including TCS, CHL, DIA, BPA, PARA, BEN, and ES, with the highest concentration tested ranging from 100 μM to 500 μM. TCS, CHL, DIA, BPS, BPA, ES, and BEN induced a significant dose-dependent decrease of cell viability (FIG. 6C). The compounds listed in FIG. 6D are the least cytotoxic, with the highest concentrations ranging from 5 mM to 50 mM. The treatments with compounds HU, VIN, TCEP, BA, and VPA had statistically significant decreases in cell viability, while CYC, TCP, ME, and SAC did not cause a significant decrease in cell viability across all concentrations.

### IC50 Values of 32 Compounds Tested Using the Co-Culture or Single Culture Models

IC50 values of the compounds treated in the co-culture or single cell culture models at 24 and 48 h are summarized in Table 2. As indicated there, IC50 values for 24 h treatment were generally higher than those for 48 h treatment. The IC50 values for Cd, ZEA, As, HEP, DES, TBBPA, TCS, HEX, and CHL were mostly ≤100 μM. The IC50 values of chemicals in the second and third groups (FIG. 6B and C) were mostly ≤500 μM. For chemicals in the fourth group (FIG. 6D), the toxicities were too low to derive IC50 values from the simulation; therefore, the highest concentrations tested were used.

TABLE 2
IC₅₀ Values of Tested Compounds among Various Cell Culture Models

| | IC50 (μM) | | | | | | | |
|---|---|---|---|---|---|---|---|---|
| | Co-culture | | Spermatogonial Cell | | Leydig Cell | | Sertoli Cell | |
| Chemical | 24 h | 48 h | 24 h | 48 h | 24 h | 48 h | 24 h | 48 h |
| ZEA | 7.2 | 4.1 | 3.8 | 2 | 3.4 | 2.5 | 3.1 | 2.5 |
| Cd | 11.3 | 4.2 | 2.1 | 2.1 | 16 | 8.5 | 14.6 | 9.5 |
| As | 15 | 10 | 6.9 | 6.8 | 20.3 | 16.1 | 12 | 6.9 |
| HEP | 19.7 | 11.4 | 109 | 20 | 2.8 | 0.6 | 3 | 2.7 |
| DES | 51.1 | 29 | 25.5 | 18 | 22.37 | 5.57 | 25.9 | 24.1 |
| TBBPA | 70 | 58 | 74.9 | 55 | 70 | 60 | 60 | 38 |
| TCS | 121 | 76.5 | 62.7 | 57.3 | 135 | 80 | 81 | 72.7 |
| BPAF | 78 | 50 | 70 | 58.5 | 30 | 20 | 55.4 | 22.3 |
| HEX | 83.1 | 82 | 22 | 5 | 69 | 59 | 55 | 44 |
| CHL | 91.5 | 89 | 118 | 96 | 142 | 120 | 212 | 120 |
| HU | 1000 | 200 | 1000 | 300 | 600 | 200 | 396 | 150 |
| BPA | 210 | 186 | 190 | 184.5 | 88 | 80.5 | 89 | 79 |
| DIA | 270 | 222 | 303 | 200 | 286 | 250 | 256 | 227 |
| BPS | 574 | 341 | 505 | 357 | 435 | 400 | 211 | 74.3 |
| DPP | 550 | 352.5 | 400 | 372 | 400 | 244 | 400 | 400 |
| DBP | 437 | 387.5 | 400 | 371 | 400 | 400 | 400 | 400 |
| PARA | 469 | 400 | 342.65 | 307.2 | 400 | 400 | 294 | 117.5 |
| TCP | 5000 | 499 | 2929 | 1867 | 5000 | 600 | 298 | 163 |
| BBP | 885 | 538.9 | 400 | 400 | 400 | 400 | 400 | 400 |
| DEHP | 681 | 400 | 527 | 400 | 400 | 400 | 400 | 400 |
| BEN | 400 | 400 | 400 | 400 | 400 | 400 | 400 | 400 |
| ES | 1000 | 240 | 1000 | 260 | 1000 | 927 | 1000 | 220 |
| VIN | 4000 | 3600 | 1788 | 560 | 2000 | 2000 | 2000 | 2000 |
| BA | 5000 | 5000 | 5000 | 3582 | 5000 | 5000 | 5000 | 5000 |
| TCEP | 5000 | 5000 | 5111 | 3678 | 5000 | 5000 | 5000 | 5000 |
| CYC | 5000 | 5000 | 5000 | 5000 | 5000 | 5000 | 5000 | 5000 |
| VPA | 20000 | 8850 | 20000 | 6000 | 20000 | 9481 | 20000 | 7606 |
| SAC | 20000 | 20000 | 20000 | 20000 | 20000 | 20000 | 20000 | 20000 |
| ME | 50000 | 50000 | 50000 | 50000 | 50000 | 50000 | 50000 | 50000 |
| DEP | 400 | 400 | 400 | 400 | 400 | 400 | 400 | 400 |
| DMP | 400 | 400 | 400 | 400 | 400 | 400 | 400 | 400 |
| DOTP | 400 | 400 | 400 | 400 | 400 | 400 | 400 | 400 |

IC50 indicates the half maximal inhibitory concentration. Cell viabilities from Neutral Red dye uptake assay were calculated as the mean value of optical density (OD) of treatment group by the control. IC50 were derived from dose-response curves with StatPlus using survival analysis and the probit method. For chemicals that the cell viability did not achieve 50% decrease at the highest concentration, the highest concentration tested was assigned as IC50.

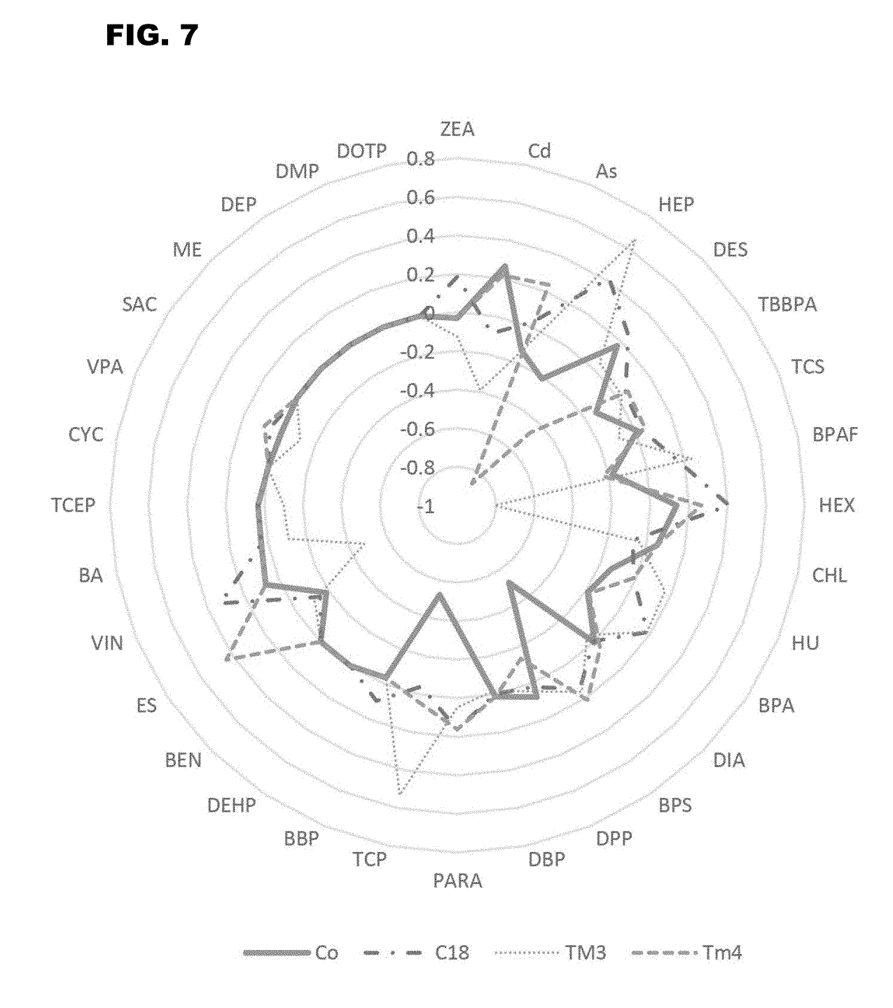

Cell type-specific difference of cytotoxicity was observed by comparing IC50 values from different cell types treated with the same compound using a radar plot, a multivariate graphical method, as shown in FIG. 7. The plot was generated based on log2 transformation IC50 values of individual compounds tested in a certain cell culture model that subtracted the average of log2 IC50 from all tested culture models. Points inside the circle indicate increased levels of toxicity (relative low IC50 values); points outside indicate less toxicity. A radar graph consists of axis lines that start in the center of a circle and extend to its periphery, and IC50 values are proportional to the radius of the graph, allowing for a direct comparison of the toxicity across cell culture conditions tested. Our results show that Sertoli cells were most sensitive to BPS and TCP (FIG. 7). Leydig cells were most sensitive to HEP and spermatogonial cells were most sensitive to HEX and VIN. Depending on the test compound, cytotoxicity obtained from the co-culture model was different from the single cell culture models, and in general, the IC50 values of the co-culture were close to the mean values of IC50 of the single cell models used (Table 2).

### Non-Supervised Cluster Analysis of IC50 Values for Co-Culture Model

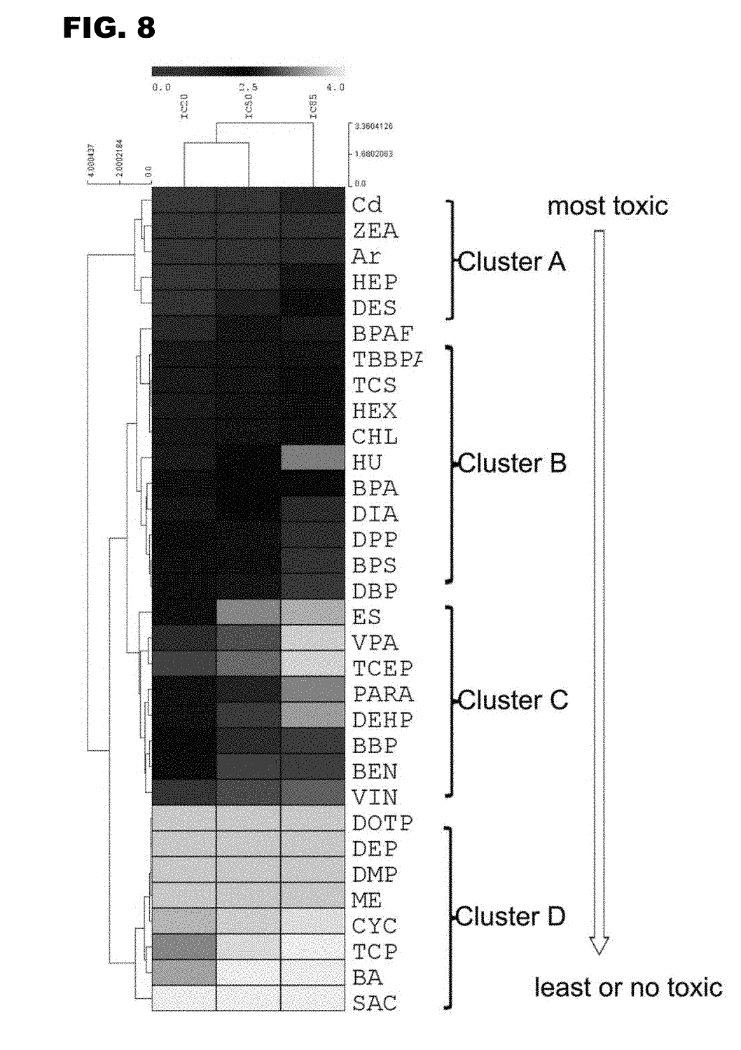

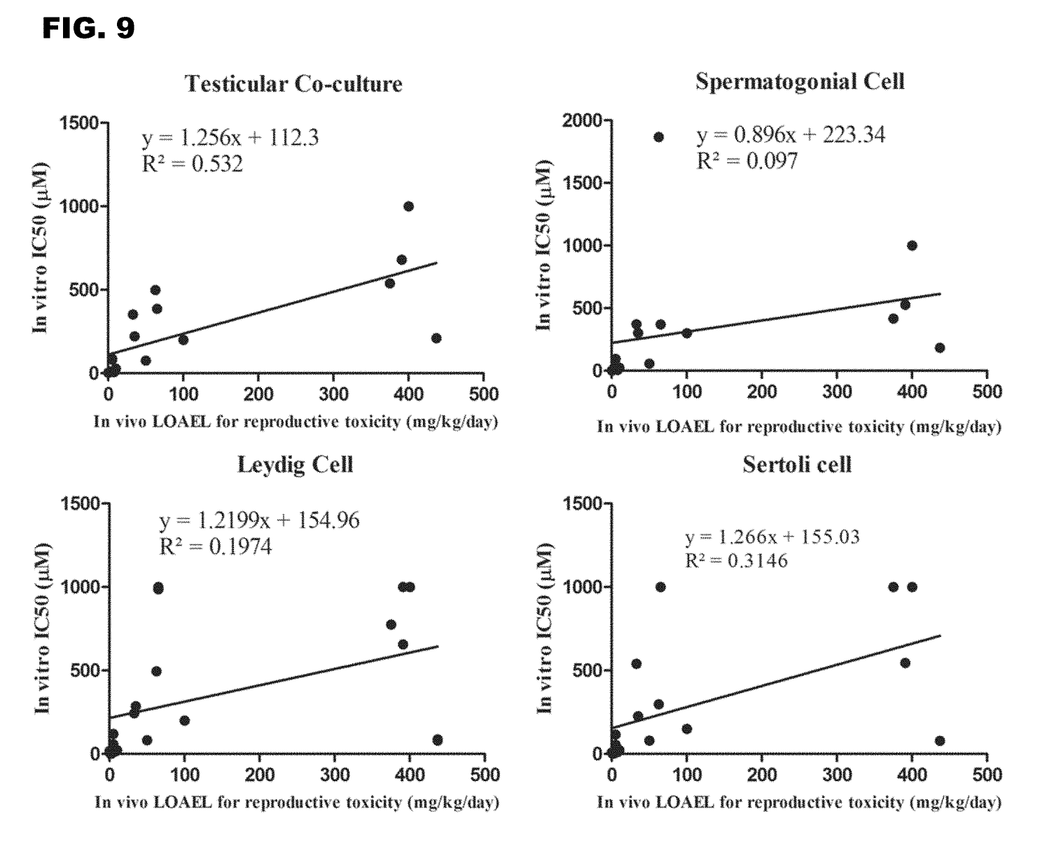

To compare the testicular toxicity among all tested chemicals using our model, a non-supervised two-dimensional hierarchical cluster analysis of IC20, IC50, and IC85 values was employed using the co-culture model for 48h treatment (FIG. 8). Based on IC20, IC50, and IC85 value obtained from cell viability, the cluster analysis organizes the IC values into discrete groups based on patterns of similarity or dis-similarity. FIG. 8 illustrates the relative degree of cell cytotoxicity in the tested compounds, which shows the cell viability in a dose-dependent manner. The chemicals at the top (cluster A) were the most toxic, and those at the bottom (cluster D) were the least toxic. Cluster A includes Cd, ZEA, As, HEP, DES; cluster B includes TBBPA, CHL, TCS, HEX, BPAF, DBP, DPP, BPA, DIA, and HU; cluster C includes BBP, BEN, VIN, PARA, DEHP, VPA, and ES; cluster D includes non-toxic phthalate esters DOTP, DEP, DMP, ME, and negative control SAC, as well as TCP, CYC and BA, which showed testicular toxicity in vivo.

Correlation Between IC50 Values from in Vitro Toxicity and in Vivo Reproductive LOAEL Values

The rLOAEL values were extracted from the published literature and indicate the lowest effective doses for reproductive toxicity in vivo. Among 32 tested compounds, 17 of them had reported in vivo rLOAEL values, and the correlation between IC50 values from in vitro and reproductive LOAEL values was examined, and determined to what extent an in vitro IC50 could predict an in vivo rLOAEL. As shown in FIG. 9, R² values for the co-culture model, spermatogonial cells, Leydig cells and Sertoli cells for 48 h treatment were 0.53, 0.1, 0.19, and 0.31, respectively. The co-culture model had the highest R² value when compared to any single cell culture model. Table 3 shows that Pearson's correlation coefficient and the r-values of the co-culture model, spermatogonial cell, Leydig cell, and Sertoli cell culture models, at 24 h treatment were 0.66, 0.16, 0.21, and 0.48, respectively; at 48h exposure, r-values were 0.73, 0.31, 0.44, and 0.56. The co-culture model displayed the highest r-value of any single cell types at both 24 h and 48 h treatments.

TABLE 3
Comparison of Correlation Coefficient among the Culture Models

| | Correlation Coefficient r | | | |
|---|---|---|---|---|
| | | Spermatogonia | | |
| Time | Co-culture | Cell | Leydig Cell | Sertoli Cell |
| 24 h | 0.6557 | 0.1632 | 0.2082 | 0.4832 |
| | (p = 0.0109) | (p = 0.5611) | p = 0.4070 | p = 0.0422 |
| 48 h | 0.7296 | 0.3114 | 0.4443 | 0.5609 |
| | (p = 0.0009) | (p = 0.2237) | (p = 0.0497) | p = 0.0192 |

### Assessment of Concordance, Sensitivity and Specificity of the Co-Culture Model

The concordance, sensitivity, and specificity were calculated for the co-culture model. Among 32 tested compounds, BPS, BPAF, and TBBPA—the analogues of BPA, tested positive in vitro and were extrapolated to be reproductive toxicants in vivo. Since there were no available in vivo data regarding their reproductive toxicity, they were not included in the calculation. Based on the results of cluster analysis, compounds in the cluster A, B and C were defined as positive reproductive toxicants, and Cluster D was defined as negative. TCP, BA, CYC, and ME in Cluster D were negative in vitro but positive in vivo models. At 48 h, the co-culture model displayed high concordance, sensitivity, and specificity, with values of 84%, 86.21%, and 100%, respectively (Table 4).

TABLE 4
Concordance, sensitivity and specificity
of the in vitro co-culture model

In vivo animal study

| | | Positive | Negative | Total |
|---|---|---|---|---|
| In vitro co-culture | Positive | 21 | 0 | 21 |
| model | Negative | 4 | 4 | 8 |
| Total | | 25 | 4 | 29 |

Sensitivity: 21/25 = 84%;
Concordance: (21 + 4)/(25 + 4) = 86.21%;
Specificity: 4/4 = 100%

### Discussion

Sertoli cells, Leydig cells, and peritubular myoid or endothelial cells all play critical roles in supporting and maintaining spermatogenesis in the testis. Damage of any type to these cells will result in testicular dysfunction. Single cell culture models of germ cells, Sertoli cells, and Leydig cells have been previously used to examine testicular toxicity (Bilinska, 1989; Chapin, et al., 1988; Gray, 1986; Griswold, 1998; Hadley, et al., 1985; Lejeune, et al., 1998; Mather, et al., 1990; Orth and Jester, 1995; Yang et al., 2003). Spermatogonial C18-4 germline cells exhibited the morphological features of spermatogonial cells and expressed germ cell-specific proteins (Hofmann, Braydich-Stolle et al. 2005). This cell line was used as an in vitro cell model to evaluate testicular toxicity (Hofmann, et al., 2005a; Hofmann, et al., 2005b; Kokkinaki et al., 2009; Liang, et al., 2017; Oatley and Brinster, 2008), determine testicular signaling pathways (Golestaneh et al., 2009; He et al., 2008; Zhang et al., 2013), and characterize the molecular mechanisms of reproductive toxicity of nanoparticles (Braydich-Stolle et al., 2005; Braydich-Stolle et al., 2010; Lucas et al., 2012). In our previous study, we developed automated multi-parametric high-content analysis (HCA) using this cell line as an in vitro model to examine the effects of Bisphenol A and its analogs on changes of cell cycle, DNA damage, and cytoskeleton. The testis has a diverse cell population, which researchers are increasingly finding useful in generating the in vitro models needed to capture the complexity of in vivo conditions. Therefore, the reconstituted co-culture models with different somatic and germ cells have become increasingly important. Primary testicular cell co-culture models have been used to evaluate testicular abnormalities during development, and have been able to identify the testicular toxicity of phthalates. However, the disadvantage of the primary testicular cell co-culture model is in employing animals for the isolation of testicular cells, and the complicated isolation procedure leads to inconsistent results. Therefore, in this study, we developed an in vitro testicular co-culture model from rodent testicular cell lines, including spermatogonial cells, Sertoli cells, and Leydig cells with specified cell density and Extracellular Matrix (ECM) composition.

Cytoskeletal proteins are known to have numerous roles, such as determination of cell shape, cell motility, maintenance of cell junctions, and intracellular trafficking to maintain normal function and morphology (Fletcher and Mullins, 2010). Spermatogenesis is a complicated process, resulting in the production of mature sperm from primordial germ cells. During spermatogenesis, significant structural and biochemical changes take place in the seminiferous epithelium of the testis, and testis-specific actin cytoskeleton plays an important role in the acquisition of mature sperm functionality during spermatogenesis and motility during fertilization (Lie et al., 2012; Lie et al., 2010). It has been shown that Sertoli cells promote the development of germ cells (Griswold, 1998; Miryounesi et al., 2013), and co-culture of germ cells with Sertoli cells in vitro could induce germ cell differentiation. Through qualitative and quantitative comparison of F-actin cytoskeletal structure between the co-culture model and single cell culture models, we found that addition of Sertoli and Leydig cells to C18-4 spermatogonia cells significantly altered their in vitro cellular structures (FIG. 1). We found that each testicular cell has its unique cytoskeletal structures (FIG. 1C-E). Two types of actin fibers were observed in C18-4 cells, including evenly distributed fine fibers inside the cytoplasm, and dense cortex F-actin on the inner boundary of the plasma membrane. Leydig cells formed diffuse F-actin fibers without a distinct boundary between cells (FIG. 1D). As demonstrated in both F-actin morphology and quantitative analysis, the actin filaments in the co-culture were organized into higher-order structures, forming actin bundles or three-dimensional networks. Through immunostaining with cell-type specific markers, we found F-actin fibers stretching across the Sertoli cell cytosol and interconnected other cells (FIG. 5). These stretching F-actin bundles, which were organized into thicker bundles, helped to form the observed cord-like structures and created an in vivo-like niche to support spermatogonial cells within a three-dimensional environment (FIG. 5). These observations suggest that the co-culture model formed a 3D cellular structure that may better resemble the in vivo physiological interactions, and achieve a better complex biological network essential for the testicular function. As revealed in our previous study, alteration of the F-actin cytoskeleton was a sensitive indicator for the cellular effects of Bisphenol A (Liang, et al., 2017). Our current results suggest that HCA-based quantitative cytoskeleton analysis with cell-type specific markers could be used as a sensitive assay to examine the effects of compounds on the testis.

As the first step to validate this in vitro co-culture model, we selected 32 compounds and applied a simple neutral red uptake assay to examine whether this in vitro co-culture model could identify the reproductive toxicants. The NR uptake assay is one of the most sensitive and reliable cytotoxicity tests (Ceridono et al., 2012; Repetto et al., 2008a). In addition, we compared the cytotoxicity from the individual cell lines, including spermatogonia C18-4, TM3, and TM4, to examine whether the in vitro co-culture model was more closely correlated with the in vivo results. We utilized the in vivo animal studies conducted by NIEHS/NTP, in which a group of experts reviewed the R/D toxicity of chemicals and identified 45 compounds as reproductive and developmental toxicants (Moorman, et al., 2000). We also drew upon the EPA's ToxCast program, which compiled the Toxicity Reference Database (ToxRefDB) including thousands of studies using a standardized approach (Auerbach, et al., 2016; Karmaus, et al., 2016; Kavlock, et al., 2012; Paul Friedman, et al., 2016). Among the 32 compounds selected for testing in the present study, 24 were confirmed in vivo reproductive toxicants and 4 of them were confirmed negative controls. Additionally, three compounds that are structure analogs of BPA, BPAF, BPS and TBBPA, were also included without in vivo information regarding their reproductive toxicity.

Using non-supervised cluster analysis of cytotoxicity, we found that the in vitro co-culture model was able to discriminate compounds into distinguishing clusters and allow the ranking of chemical toxicity. Compared with published in vivo studies, our in vitro results were consistent overall, with in vivo toxicity data with a concordance of 86.2% and specificity of 83.3%. The reproductive LOAEL values of Cd, ZEA, As, DES, and HEP in rats were 0.088, 1, 8, 10, and 3 mg/kg/day, respectively (Martin, et al., 2011; Seiler and Spielmann, 2011). DEP, DOTP, and DMP—the least toxic group—are well known as developmental non-toxic phthalates (Gray et al., 2000; Yu, et al., 2009). SAC is a sweetener and was used as a negative reference compound. By comparing the current model with our previous primary cells co-culture model (Yu, et al., 2009), we found that both our cell line model and the primary co-culture model can distinguish the toxic phthalates (DEHP, DBP, BBP, and DPP) from the non-toxic phthalates (DEP, DMP and DOTP). We observed a higher cell death in the current cell line model than that of the previous primary cell model at the same concentrations for cadmium treatment (Yu, et al., 2005). Cluster analysis can help to predict the toxicity level of new chemicals based on the cluster in which the chemical resides. TBBPA, BPS, and BPAF are analogues of BPA, and emerged as alternatives for BPA. So far, there is still insufficient in vivo toxicological data regarding their reproductive toxicity. Based on the results of their positions in the cluster analysis, as well as their structure similarity to BPA, these compounds are predicted to be reproductive toxicants. This prediction was reinforced by the current ongoing in vivo study from the National Toxicology Program (NTP), which showed that BPAF exposure uniquely impaired pregnancies and sexual development in rats (Sutherland. et al., 2017).

Through comparing the cytotoxicity data from the co-culture model and the single cell culture models, we found the co-culture model had the highest correlation with the in vivo data. Our results suggest that the co-culture model thus offered a better predictive power than the single cell culture models. Different cell types displayed different sensitivities to the same chemical, as indicated by IC50 values (Table 2 and FIG. 7). Sertoli cells, Leydig cells, and spermatogonial cells in the testis play differential roles in regulation of spermatogenesis. The use of single cell culture models may not reflect the in vivo responses, but could help elucidate the cell type-specific effect as well as the underlying mechanisms of toxicants. For example, the Leydig cell model is often used to investigate the effect of chemicals on steroidogenesis (Forgacs et al., 2012). In our current study, we found both Leydig cells and Sertoli cells were more sensitive to HEP as compared to spermatogonia and co-culture models, suggesting that Leydig cells and Sertoli could be a sensitive target for HEP. In fact, HEP is reported to induced testicular toxicity targeting the Sertoli cells (James et al., 1980) and significant decrease of inhibin B, an biomarker for Sertoli cell, was reported (Erdos et al., 2013). The lower IC50 values of TCP and BPS in the Sertoli cell model indicated that Sertoli cells appeared to be the target for these compounds. These findings are linked with previous studies, which reported that TCP damaged the blood-testis barrier (Sertoli cells), elicited subsequent damage to germ cells, and caused germ cell loss (Chapin et al., 1991). There are increasing concerns about the potential toxic effects of bisphenol analogs such as BPS and BPAF (Liang et al., 2016). There is lack of toxicological data of BPS, and it is unclear whether BPS targets the Sertoli cells in vivo animal. Thus, comparing toxicity data from various single cell culture models might allow us to evaluate cell-type specific responses in the testis, but might not correlate well with the in vivo condition. The co-culture model, which enables cell-cell communication among various cell types, should be a better system than any single cell type to screen testicular toxic chemicals.

Although our current in vitro co-culture provided valuable information on the potential toxicity of chemicals, it also demonstrated the limitations commonly shared by in vitro cell based assays. For example, chemicals that require metabolic activation, such as TCP, BA, CYC, and ME, will not be predicted as reproductive toxicants using this model. ME has been reported to induce testicular atrophy and disrupt spermatogenesis via the metabolism of Ethylene glycol monomethyl ether (EGME) (Foster et al., 1984; Foster et al., 1983; Starek-Swiechowicz et al., 2015; van der Laan et al., 2012). It was found that ME undergoes metabolic activation to appropriate methoxyacetic acid (MAA) via EGME (Takei et al., 2010). MAA primarily affects tissues with rapidly dividing cells and high rates of energy metabolism in the testes, leading to apoptosis of primary spermatocytes. Spermatogenesis is a multi-step complex process. Our current in vitro co-culture model captured an early stage of spermatogenesis and will probably not capture all the toxicologically-vulnerable processes in the testis. Chemicals such as boric acid would likely cause male reproductive toxicity through nonmolecular interactions, and lead to damage of developing spermatids (Chapin and Ku, 1994; Jewell et al., 1998). Similarly, VIN treatment from gestation day 15 to postnatal week 4 at the concentration of 7.2 and 72 mg/kg/day has induced abnormal spermatozoa with nuclear and acrosomal defects (Veeramachaneni et al., 2006). Therefore, further efforts are needed in order to differentiate spermatogonia in the co-culture model to produce post-meiotic spermatids as observed in explanted pieces of testis (Brannen, et al., 2016; Sato, et al., 2011). Thus, it is unlikely that any individual in vitro model is sufficient as a final decision point for male reproductive toxicity; rather, a tiered approach using a series of models containing multiple endpoint analysis and concentration response curves is essential to building a robust screening platform to improve the prediction accuracy of in vitro assays.

Animal studies could provide credible evidence to predict the likely effect of chemical exposure on human outcomes. However, the toxicity testing from the animal studies predicted toxicity in humans with only about 50-70% accuracy (Chapin et al., 2013; Olson et al., 2000). Our current results represent an improvement over previous attempts to predict reproductive toxicity responses. The doses selected for these compounds were initially determined based on the literature and adjusted to ensure derivation of the IC values from the dose-response curves (FIG. 6). The in vitro assay can be optimized to have better relevance with in vivo. For example, the concentration obtained from the in vitro should reflect the plasma concentration attained at the lowest dose that produces toxicity in humans. In order to fully use these in vitro data, we need to develop a physiological based toxicokinetic model (PBTK) to extrapolate the in vivo exposure as well as differences in the metabolism.

In summary, by utilizing testicular cell lines we constructed a testicular cell co-culture model, demonstrated the formation of a three-dimensional cytoskeleton structure, and were able to distinguish testicular toxic compounds from non-toxic chemicals. Moreover, the toxicity in the co-culture model at 48 h was found to have the highest correlation with rLOAEL in vivo. The calculation of concordance, sensitivity, and specificity further supported the reliability of this model. Our results suggest that our in vitro co-culture model may be useful in screening testicular toxicants in a wide concentration range and prioritizing chemicals for further assessment. Furthermore, the exploitation of high-content imaging and quantitative techniques provides deep insight into the molecular and cellular mechanisms. In future research, we will include more compounds to further validate this in vitro co-culture model, and establish and examine more endpoints that correlate with different adverse outcomes of in vivo reproductive toxicity.

### REFERENCES

Auerbach, S., Filer, D., Reif, D., Walker, V., Holloway, A. C., Schlezinger, J., Srinivasan, S., Svoboda, D., Judson, R., Bucher, J. R., et al. (2016). Prioritizing Environmental Chemicals for Obesity and Diabetes Outcomes Research: A Screening Approach Using ToxCast High Throughput Data. Environ Health Perspect doi: 10.1289/ehp.1510456.

Bilinska, B. (1989). Interaction between Leydig and Sertoli cells in vitro. Cytobios 60(241), 115-26.

Brannen, K. C., Chapin, R. E., Jacobs, A. C., and Green, M. L. (2016). Alternative Models of Developmental and Reproductive Toxicity in Pharmaceutical Risk Assessment and the 3Rs. ILAR J 57(2), 144-156.

Braydich-Stolle, L., Hussain, S., Schlager, J. J., and Hofmann, M. C. (2005). In vitro cytotoxicity of nanoparticles in mammalian germline stem cells. Toxicological Sciences 88(2), 412-419.

Braydich-Stolle, L. K., Lucas, B., Schrand, A., Murdock, R. C., Lee, T., Schlager, J. J., Hussain, S. M., and Hofmann, M. C. (2010). Silver Nanoparticles Disrupt GDNF/Fyn kinase Signaling in Spermatogonial Stem Cells. Toxicological Sciences 116(2), 577-589.

Casey, W. M. (2016). Advances in the Development and Validation of Test Methods in the United States. Toxicol Res 32(1), 9-14.

Ceridono, M., Tellner, P., Bauer, D., Barroso, J., Alépée, N., Corvi, R., De Smedt, A., Fellows, M. D., Gibbs, N. K., Heisler, E., et al. (2012). The 3T3 neutral red uptake phototoxicity test: Practical experience and implications for phototoxicity testing—The report of an ECVAM—EFPIA workshop. Regulatory Toxicology and Pharmacology 63(3), 480-488.

Chapin, R. E., Boekelheide, K., Cortvrindt, R., van Duursen, M. B. M., Gant, T., Jegou, B., Marczylo, E., van Pelt, A. M. M., Post, J. N., Roelofs, M. J. E., et al. (2013). Assuring safety without animal testing: The case for the human testis in vitro. Reproductive Toxicology 39, 63-68.

Chapin, R. E., Gray, T. J., Phelps, J. L., and Dutton, S. L. (1988). The effects of mono-(2-ethylhexyl)-phthalate on rat Sertoli cell-enriched primary cultures. Toxicol Appl Pharmacol 92(3), 467-79.

Chapin, R. E., and Ku, W. W. (1994). The reproductive toxicity of boric acid. Environ Health Perspect 102 Suppl 7, 87-91.

Chapin, R. E., Phelps, J. L., Burka, L. T., Abou-Donia, M. B., and Heindel, J. J. (1991). The effects of tri-o-cresyl phosphate and metabolites on rat Sertoli cell function in primary culture. Toxicol Appl Pharmacol 108(2), 194-204.

Chapin, R. E., and Stedman, D. B. (2009). Endless possibilities: stem cells and the vision for toxicology testing in the 21st century. Toxicological sciences : an official journal of the Society of Toxicology 112(1), 17-22.

Chittenden, T. W., Howe, E. A., Taylor, J. M., Mar, J. C., Aryee, M. J., Gomez, H., Sultana, R., Braisted, J., Nair, S. J., Quackenbush, J., et al. (2012). nEASE: a method for gene ontology subclassification of high-throughput gene expression data. Bioinformatics 28(5), 726-8.

CIRM (2008). Stem Cells in Predictive Toxicology CIRM Workshop Report.

Clark, B. J., Wells, J., King, S. R., and Stocco, D. M. (1994). The purification, cloning, and expression of a novel luteinizing hormone-induced mitochondrial protein in MA-10 mouse Leydig tumor cells. Characterization of the steroidogenic acute regulatory protein (StAR). J Biol Chem 269(45), 28314-22.

Enders, G. C., and May, J. J., 2nd (1994). Developmentally regulated expression of a mouse germ cell nuclear antigen examined from embryonic day 11 to adult in male and female mice. Dev Biol 163(2), 331-40.

Erdos, Z., Pearson, K., Goedken, M., Menzel, K., Sistare, F. D., Glaab, W. E., and Saldutti, L. P. (2013). Inhibin B response to testicular toxicants hexachlorophene, ethane dimethane sulfonate, di-(n-butyl)-phthalate, nitrofurazone, DL-ethionine, 17-alpha ethinylestradiol, 2,5-hexanedione, or carbendazim following short-term dosing in male rats. Birth defects research. Part B, Developmental and reproductive toxicology 98(1), 41-53.

Fletcher, D. A., and Mullins, R. D. (2010). Cell mechanics and the cytoskeleton. Nature 463(7280), 485-92.

Forgacs, A. L., Ding, Q., Jaremba, R. G., Huhtaniemi, I. T., Rahman, N. A., and Zacharewski, T. R. (2012). BLTK1 murine Leydig cells: a novel steroidogenic model for evaluating the effects of reproductive and developmental toxicants. Toxicological sciences : an official journal of the Society of Toxicology 127(2), 391-402.

Foster, P. M., Creasy, D. M., Foster, J. R., and Gray, T. J. (1984). Testicular toxicity produced by ethylene glycol monomethyl and monoethyl ethers in the rat. Environ Health Perspect 57, 207-17.

Foster, P. M., Creasy, D. M., Foster, J. R., Thomas, L. V., Cook, M. W., and Gangolli, S. D. (1983). Testicular toxicity of ethylene glycol monomethyl and monoethyl ethers in the rat. Toxicol Appl Pharmacol 69(3), 385-99.

Golestaneh, N., Beauchamp, E., Fallen, S., Kokkinaki, M., Uren, A., and Dym, M. (2009). Wnt signaling promotes proliferation and stemness regulation of spermatogonial stem/progenitor cells. Reproduction 138(1), 151-162.

Gray, L. E., Jr., Ostby, J., Furr, J., Price, M., Veeramachaneni, D. N., and Parks, L. (2000). Perinatal exposure to the phthalates DEHP, BBP, and DINP, but not DEP, DMP, or DOTP, alters sexual differentiation of the male rat. Toxicological sciences : an official journal of the Society of Toxicology 58(2), 350-65.

Gray, T. J. (1986). Testicular toxicity in vitro: Sertoli-germ cell co-cultures as a model system. Food Chem Toxicol 24(6-7), 601-5.

Griswold, M. D. (1998). The central role of Sertoli cells in spermatogenesis. Semin Cell Dev Biol 9(4), 411-6.

Hadley, M. A., Byers, S. W., Suarez-Quian, C. A., Kleinman, H. K., and Dym, M. (1985). Extracellular matrix regulates Sertoli cell differentiation, testicular cord formation, and germ cell development in vitro. J Cell Biol 101(4), 1511-22.

Hareng, L., Pellizzer, C., Bremer, S., Schwarz, M., and Hartung, T. (2005). The integrated project ReProTect: a novel approach in reproductive toxicity hazard assessment. Reprod Toxicol 20(3), 441-52.

Harris, S., Hermsen, S. A., Yu, X., Hong, S. W., and Faustman, E. M. (2015). Comparison of toxicogenomic responses to phthalate ester exposure in an organotypic testis co-culture model and responses observed in vivo. Reprod Toxicol 58, 149-59.

Hartung, T., and Rovida, C. (2009). Chemical regulators have overreached. Nature 460(7259), 1080-1.

He, Z. P., Jiang, J. J., Kokkinaki, M., Golestaneh, N., Hofmann, M. C., and Dym, M. (2008). GDNF upregulates c-fos transcription via the Ras/ERK1/2 pathway to promote mouse spermatogonial stem cell proliferation. Stem Cells 26(1), 266-278.

Hofmann, M. C., Braydich-Stolle, L., Dettin, L., Johnson, E., and Dym, M. (2005a). Immortalization of mouse germ line stem cells. Stem Cells 23(2), 200-10.

Hofmann, M. C., Braydich-Stolle, L., and Dym, M. (2005b). Isolation of male germ-line stem cells; influence of GDNF. Dev Biol 279(1), 114-124.

Hofstetter, E., Spencer, J. W., Hiteshew, K., Coutu, M., and Nealley, M. (2013). Evaluation of recommended REACH exposure modeling tools and near-field, far-field model in assessing occupational exposure to toluene from spray paint. Ann Occup Hyg 57(2), 210-20.

Houck, K. A., Dix, D. J., Judson, R. S., Kavlock, R. J., Yang, J., and Berg, E. L. (2009). Profiling bioactivity of the ToxCast chemical library using BioMAP primary human cell systems. J Biomol Screen 14(9), 1054-66.

ICCVAM (2012). ICCVAM recommends in vitro test method for endocrine-disruptors. Altern Lab Anim 40(1), 11.

James, R. W., Heywood, R., and Crook, D. (1980). Quantitative aspects of spermatogenesis in rats and dogs after repeated hexachlorophene treatment. Toxicol Lett 5(6), 405-12.

Jenardhanan, P., Panneerselvam, M., and Mathur, P. P. (2016). Effect of environmental contaminants on spermatogenesis. Semin Cell Dev Biol 59, 126-140.

Jewell, W. T., Hess, R. A., and Miller, M. G. (1998). Testicular toxicity of molinate in the rat: metabolic activation via sulfoxidation. Toxicol Appl Pharmacol 149(2), 159-66.

Karmaus, A. L., Filer, D. L., Martin, M. T., and Houck, K. A. (2016). Evaluation of food-relevant chemicals in the ToxCast high-throughput screening program. Food and Chemical Toxicology 92, 188-96.

Kavlock, R., Chandler, K., Houck, K., Hunter, S., Judson, R., Kleinstreuer, N., Knudsen, T., Martin, M., Padilla, S., Reif, D., et al. (2012). Update on EPA's ToxCast Program: Providing High Throughput Decision Support Tools for Chemical Risk Management. Chemical Research in Toxicology 25(7), 1287-1302.

Kokkinaki, M., Lee, T. L., He, Z. P., Jiang, J. J., Golestaneh, N., Hofmann, M. C., Chan, W. Y., and Dym, M. (2009). The Molecular Signature of Spermatogonial Stem/Progenitor Cells in the 6-Day-Old Mouse Testis. Biology of Reproduction 80(4), 707-717.

Lejeune, H., Sanchez, P., and Saez, J. M. (1998). Enhancement of long-term testosterone secretion and steroidogenic enzyme expression in human Leydig cells by co-culture with human Sertoli cell-enriched preparations. Int J Androl 21(3), 129-40.

Leung, M. C., Phuong, J., Baker, N. C., Sipes, N. S., Klinefelter, G. R., Martin, M. T., McLaurin, K. W., Setzer, R. W., Darney, S. P., Judson, R. S., et al. (2016). Systems Toxicology of Male Reproductive Development: Profiling 774 Chemicals for Molecular Targets and Adverse Outcomes. Environ Health Perspect 124(7), 1050-61.

Liang, S., Yin, L., Shengyang Yu, K., Hofmann, M. C., and Yu, X. (2017). High-Content Analysis Provides Mechanistic Insights into the Testicular Toxicity of Bisphenol A and Selected Analogues in Mouse Spermatogonial Cells. Toxicol Sci 155(1), 43-60.

Liang, S., Yin, L., Shengyang Yu, K., Hofmann, M. C., and Yu, X. (2016). High-Content Analysis Provides Mechanistic Insights into the Testicular Toxicity of Bisphenol A and Selected Analogues in Mouse Spermatogonial Cells. Toxicological sciences: an official journal of the Society of Toxicology doi: 10.1093/toxsci/kfw178.

Lie, P. P., Cheng, C. Y., and Mruk, D. D. (2012). The biology of interleukin-1: emerging concepts in the regulation of the actin cytoskeleton and cell junction dynamics. Cell Mol Life Sci 69(4), 487-500.

Lie, P. P., Mruk, D. D., Lee, W. M., and Cheng, C. Y. (2010). Cytoskeletal dynamics and spermatogenesis. Philos Trans R Soc Lond B Biol Sci 365(1546), 1581-92.

Lucas, B. E., Fields, C., Joshi, N., and Hofmann, M. C. (2012). Mono-(2-ethylhexyl)-phthalate (MEHP) affects ERK-dependent GDNF signalling in mouse stem-progenitor spermatogonia. Toxicology 299(1), 10-9.

Luijten, M., de Vries, A., Opperhuizen, A., and Piersma, A. H. (2007). Alternative methods in reproductive toxicity testing: state of the art. RIVM report, 340720002.

Martin, M. T., Knudsen, T. B., Reif, D. M., Houck, K. A., Judson, R. S., Kavlock, R. J., and Dix, D. J. (2011). Predictive model of rat reproductive toxicity from ToxCast high throughput screening. Biol Reprod 85(2), 327-39.

Mather, J. P. (1980). Establishment and characterization of two distinct mouse testicular epithelial cell lines. Biol Reprod 23(1), 243-52.

Mather, J. P., Attie, K. M., Woodruff, T. K., Rice, G. C., and Phillips, D. M. (1990). Activin stimulates spermatogonial proliferation in germ-Sertoli cell cocultures from immature rat testis. Endocrinology 127(6), 3206-14.

Mather, J. P., and Phillips, D. M. (1984). Establishment of a peritubular myoid-like cell line and interactions between established testicular cell lines in culture. J Ultrastruct Res 87(3), 263-74.

Miryounesi, M., Nayernia, K., Dianatpour, M., Mansouri, F., and Modarressi, M. H. (2013). Co-culture of Mouse Embryonic Stem Cells with Sertoli Cells Promote in vitro Generation of Germ Cells. Iran J Basic Med Sci 16(6), 779-83.

Moorman, W. J., Ahlers, H. W., Chapin, R. E., Daston, G. P., Foster, P. M., Kavlock, R. J., Morawetz, J. S., Schnorr, T. M., and Schrader, S. M. (2000). Prioritization of NTP reproductive toxicants for field studies. Reprod Toxicol 14(4), 293-301.

Oatley, J. M., and Brinster, R. L. (2008). Regulation of spermatogonial stem cell self-renewal in mammals. Annu Rev Cell Dev Biol 24, 263-86.

OECD (2016). OECD Guidelines for the Testing of Chemicals.

Olson, H., Betton, G., Robinson, D., Thomas, K., Monro, A., Kolaja, G., Lilly, P., Sanders, J., Sipes, G., Bracken, W., et al. (2000). Concordance of the toxicity of pharmaceuticals in humans and in animals. Regul Toxicol Pharmacol 32(1), 56-67.

Orth, J. M., and Jester, W. F., Jr. (1995). NCAM mediates adhesion between gonocytes and Sertoli cells in cocultures from testes of neonatal rats. J Androl 16(5), 389-99.

Parks Saldutti, L., Beyer, B. K., Breslin, W., Brown, T. R., Chapin, R. E., Campion, S., Enright, B., Faustman, E., Foster, P. M., Hartung, T., et al. (2013). In vitro testicular toxicity models: Opportunities for advancement via biomedical engineering techniques. ALTEX 30(3), 353-77.

Paul Friedman, K., Watt, E. D., Hornung, M. W., Hedge, J. M., Judson, R. S., Crofton, K. M., Houck, K. A., and Simmons, S. O. (2016). Tiered High-Throughput Screening Approach to Identify Thyroperoxidase Inhibitors Within the ToxCast Phase I and II Chemical Libraries. Toxicological Sciences 151(1), 160-80.

Reif, D. M., Martin, M. T., Tan, S. W., Houck, K. A., Judson, R. S., Richard, A. M., Knudsen, T. B., Dix, D. J., and Kavlock, R. J. (2010). Endocrine profiling and prioritization of environmental chemicals using ToxCast data. Environ Health Perspect 118(12), 1714-20.

Repetto, G., del Peso, A., and Zurita, J. L. (2008a). Neutral red uptake assay for the estimation of cell viability/cytotoxicity. Nature protocols 3(7), 1125-31.

Repetto, G., del Peso, A., and Zurita, J. L. (2008b). Neutral red uptake assay for the estimation of cell viability/cytotoxicity. Nature Protocols 3(7), 1125-1131.

Sato, T., Katagiri, K., Gohbara, A., Inoue, K., Ogonuki, N., Ogura, A., Kubota, Y., and Ogawa, T. (2011). In vitro production of functional sperm in cultured neonatal mouse testes. Nature 471(7339), 504-7.

Sauer, U. G. (2004). Avoidance of animal experiments in the new EU Chemicals Regulation—opportunities and problems from the point of view of animal welfare. Altex 21(1), 9-14.

Scialli, A. R., and Guikema, A. J. (2012). REACH and reproductive and developmental toxicology: still questions. Syst Biol Reprod Med 58(1), 63-9.

Seiler, A. E., and Spielmann, H. (2011). The validated embryonic stem cell test to predict embryotoxicity in vitro. Nat Protoc 6(7), 961-78.

Spade, D. J., Hall, S. J., Saffarini, C. M., Huse, S. M., McDonnell, E. V., and Boekelheide, K. (2014a). Differential response to abiraterone acetate and di-n-butyl phthalate in an androgen-sensitive human fetal testis xenograft bioassay. Toxicological sciences: an official journal of the Society of Toxicology 138(1), 148-60.

Spade, D. J., McDonnell, E. V., Heger, N. E., Sanders, J. A., Saffarini, C. M., Gruppuso, P. A., De Paepe, M. E., and Boekelheide, K. (2014b). Xenotransplantation models to study the effects of toxicants on human fetal tissues. Birth defects research. Part B, Developmental and reproductive toxicology 101(6), 410-22.

Starek-Swiechowicz, B., Szymczak, W., Budziszewska, B., and Starek, A. (2015). Testicular effect of a mixture of 2-methoxyethanol and 2-ethoxyethanol in rats. Pharmacological reports: PR 67(2), 289-93.

Sutherland., V., McIntyre., B., Pelch., K., Waidyanatha., S., Conley., J. M., Gray., L. E., and Foster, P. M. A Comparison of In Vivo Reproductive and Developmental Toxicity (DART) Endpoints for Bisphenol AF and Bisphenol A2017, Baltimore, Md.

Takei, M., Ando, Y., Saitoh, W., Tanimoto, T., Kiyosawa, N., Manabe, S., Sanbuissho, A., Okazaki, O., Iwabuchi, H., Yamoto, T., et al. (2010). Ethylene glycol monomethyl ether-induced toxicity is mediated through the inhibition of flavoprotein dehydrogenase enzyme family. Toxicological sciences: an official journal of the Society of Toxicology 118(2), 643-52.

ThermoFisher (2010). Thermo Scientific Cellomics®Morphology Explorer V4 BioApplication Guide. In (Thermo Fisher Scientific Inc. 100 Technology Drive Pittsburgh, Penn. 15219.

van der Laan, J. W., Chapin, R. E., Haenen, B., Jacobs, A. C., and Piersma, A. (2012). Testing strategies for embryo-fetal toxicity of human pharmaceuticals. Animal models vs. in vitro approaches: a workshop report. Regul Toxicol Pharmacol 63(1), 115-23.

Veeramachaneni, D. N., Palmer, J. S., Amann, R. P., Kane, C. M., Higuchi, T. T., and Pau, K. Y. (2006). Disruption of sexual function, FSH secretion, and spermiogenesis in rabbits following developmental exposure to vinclozolin, a fungicide. Reproduction 131(4), 805-16.

Wegner, S., Hong, S., Yu, X., and Faustman, E. M. (2013). Preparation of rodent testis co-cultures. Current protocols in toxicology/editorial board, Mahin D. Maines Chapter 16, Unit 16 10.

Wegner, S. H., Yu, X., Kim, H., Harris, S., Griffith, W. C., Hong, S. H., and Faustman, E. M. (2014). Effect of dipentyl phthalate in 3-dimensional in vitro testis co-culture is attenuated by cyclooxygenase-2 inhibition. JTEHS 6(8), 161-169.

Yang, J. M., Arnush, M., Chen, Q. Y., Wu, X. D., Pang, B., and Jiang, X. Z. (2003). Cadmium-induced damage to primary cultures of rat Leydig cells. Reprod Toxicol 17(5), 553-60.

Yu, X., Hong, S., and Faustman, E. M. (2008). Cadmium-induced activation of stress signaling pathways, disruption of ubiquitin-dependent protein degradation and apoptosis in primary rat Sertoli cell-gonocyte cocultures. Toxicological sciences: an official journal of the Society of Toxicology 104(2), 385-96.

Yu, X., Hong, S., Moreira, E. G., and Faustman, E. M. (2009). Improving in vitro Sertoli cell/gonocyte co-culture model for assessing male reproductive toxicity: Lessons learned from comparisons of cytotoxicity versus genomic responses to phthalates. Toxicol Appl Pharmacol 239(3), 325-36.

Yu, X., Sidhu, J. S., Hong, S., and Faustman, E. M. (2005). Essential role of extracellular matrix (ECM) overlay in establishing the functional integrity of primary neonatal rat Sertoli cell/gonocyte co-cultures: an improved in vitro model for assessment of male reproductive toxicity. Toxicological sciences : an official journal of the Society of Toxicology 84(2), 378-93.

Zhang, Z. Z., Gong, Y. H., Guo, Y., Hai, Y. N., Yang, H., Yang, S., Liu, Y., Ma, M., Liu, L. H., Li, Z., et al. (2013). Direct transdifferentiation of spermatogonial stem cells to morphological, phenotypic and functional hepatocyte-like cells via the ERK1/2 and Smad2/3 signaling pathways and the inactivation of cyclin A, cyclin B and cyclin E. Cell Commun Signal 11.

### Example 2

### Machine Learning-Powered High Content Analysis to Characterize Phenotypes Associated with Toxicity of Bisphenol A and its Analogs Bisphenol S, Bisphenol AF, and Tetrabromobisphenol A in a Testicular Cell Co-Culture Model

High-content analysis (HCA) has emerged as a powerful tool for chemical toxicity profiling. A multi-parametric HCA in a spermatogonial cell line was previously developed to examine the testicular toxicities of Bisphenol A (BPA) and its Analogs: bisphenol S (BPS), bisphenol AF (BPAF), and tetrabromobisphenol A (TBBPA). Due to the complexity of high-dimensional and large-scale HCA data, there is an increased demand for effective computational strategies to characterize and quantify phenotypic effects at a single-cell level. Here we describe the development of a machine learning (ML)-based HCA pipeline and explore the complex phenotypic changes. This pipeline allowed us to characterize the toxicity of BPA and its Analogs in a testicular co-culture model using spermatogonial, Leydig and Sertoli cell lines. The use of a

ML-based phenotypic analysis allowed us to observe that the treatments of BPA or its Analogs resulted in the loss of spatial arrangement of the model's three-dimensional (3D) structure and an accumulation of cells in M phase arrest in a dose- and time-dependent manner. Furthermore, BPAF induced an accumulation of multinucleated cells, which was associated with an increase of DNA damage response, and impairment in cellular actin filaments. These results showed that BPAF and TBBPA exerted toxicity at lower doses than BPA and BPS on multiple endpoints in the co-culture model. In summary, we have developed a ML-based HCA approach in the testicular cell co-culture model that reflected complex phenotypic changes and characterized the testicular toxicities of BPA and its Analogs. This approach provided an in-depth analysis of multi-dimensional HCA data and unbiased quantitative analysis of the phenotypes of interest.

### Introduction

With recent advances in automated fluorescence microscopy and the development of quantitative image analysis software, high-content analysis (HCA) enables measurements of unbiased multi-parametric data on a single-cell level and provides both temporal and spatial measurements of various cellular events associated with adverse health outcomes (Buchser et al., 2004; Mattiazzi Usaj et al., 2016; Zanella et al., 2010). This approach has been used to prioritize chemical toxicity for further studies, to characterize adverse outcome pathways, and to develop predictive models for toxicity evaluation in humans (Elmore et al., 2014; Merrick et al., 2015; Shukla et al., 2010). The U.S. Environmental Protection Agency (EPA) initiated the ToxCast program using in vitro high-throughput and high-content screening (HTS and HCS) to profile and predict the toxicity of thousands of environmental chemicals (Krewski et al., 2010; Martin et al., 2011; Paul Friedman et al., 2016). While HCA provides large-scale, image-based data, the analysis of these data becomes a major bottleneck as the quantification of cellular phenotypes usually depends on manual parameter adjustments, which is both time- and labor-intensive and has low reproducibility among different experiments (Sommer and Gerlich, 2013). Thus, the demand for advanced computational strategies that explore the inherent structure of multidimensional data, and provide an unbiased assessment of a variety of phenotypes in large-scale image data sets. Supervised machine learning (ML) has emerged as a powerful approach in classifying cellular heterogeneity using non-linear multi-parametric algorithms for HCA data (Altschuler and Wu, 2010; Sommer and Gerlich, 2013). These algorithms are then able to learn from the small training datasets labeled with predefined classes and then automatically infer the rules to classify full datasets.. This application of machine learning has been applied in the examiniation of the dynamic changes of the genome and the proteome in single cell imaging (Chong et al., 2015; Neumann et al., 2010), its application in toxicology, however, has not fully been explored.

In a previous study, our lab established and validated a battery of HCA assays using spermatogonial cell line C18-4 and revealed the different testicular toxicities of Bisphenol A (BPA) and its Analogs: bisphenol S (BPS), bisphenol AF (BPAF) and tetrabromobisphenol A (TBBPA) (Liang et al., 2017b). BPA is a high production-volume chemical widely used in consumer products, thermal paper, medical devices, and dental sealants (Rochester, 2013). Exposure to BPA is widespread and occurs mainly through ingestion, inhalation and dermal exposures (Kang et al., 2006; Vandenberg et al., 2007). BPA has been detected in over 90% of urine samples from the general population in the United States. (Calafat et al., 2008; Lakind and Naiman, 2011). BPA is a well-studied endocrine disruptor, as it has been determined as a reproductive and developmental toxicant in animal models (Peretz et al., 2014; Richter et al., 2007; Rochester, 2013; vom Saal et al., 2007). Due to the concern of ubiquitous exposure to BPA and its potential adverse effects on humans, the U.S. Food, and Drug Administration (FDA) and European Chemical Agency (ECHA) have placed restrictions on the use of BPA (EU., 2016; FDA., 2013; FDA., 2012). The structural Analogs of BPA have been introduced into the market as a BPA substitute and share similar manufacturing applications to BPA. Although there is a general lack of production data for BPA Analogs, the usage of these chemicals is expected to rise globally. Due to high structural similarities, the Analogs could potentially exhibit estrogenic potencies and reproductive toxicities comparable with BPA. Emerging evidence indicates that BPA Analogs have been found in food and human urine samples and interacted with various physiological receptors (Driffield et al., 2008; Kitamura et al., 2005; Liao and Kannan, 2013; Liao et al., 2012; Stossi et al., 2014). However, toxicological data concerning BPA Analogs are still limited.

In this study, we applied a testicular cell co-culture model to examine the testicular toxicities of BPA and its Analogs, which provided a more closely approximated in vivo testicular microenvironment encountered with environmental exposures. The testicular cell co-culture model was able to recapitulate the multicellular complexity and organ-like structure, and mimic the physiology relevant to vivo. Sertoli and Leydig cells play critical roles in regulating and supporting spermatogenesis and maintaining the structure and functions of the testis (Haywood et al., 2003; Lui et al., 2003; Payne, 1990). The primary testicular cell co-culture model employed germ, Sertoli, and Leydig cells with an extracellular matrix (ECM) was reported to exhibit testicle-like multilayered architecture. This model demonstrated the differential metabolic capacity, inflammatory responses, and differential toxicogenomic responses to phthalate exposures (Harris et al., 2015; Harris et al., 2016a; Harris et al., 2016b; Yu et al., 2009; Yu et al., 2005). Recently, we developed an animal-free, in vitro testicular cell co-culture model utilizing mouse spermatogonial (C18-4), Sertoli (TM4), and Leydig (TM3) cell lines, which exhibited a unique three-dimensional (3D) in vivo structure compared to single cell cultures, and enabled us to classify the reproductive toxic substances with high specificity and sensitivity (Example 1).

The purpose of this study was to develop a supervised ML-based HCA to characterize phenotypes associated with the testicular toxicities of BPA and its structural Analogs: BPS, BPAF, and TBBPA in a testicular cell co-culture model. We measured a wide spectrum of adverse endpoints, which characterized individual cells from different phenotypes in testicular cells, including nuclear morphology, DNA synthesis, DNA damage, and cytoskeleton structure using the CellProfiler Analyst. Our lab has developed a phenotype recognition pipeline, comprised of image pre-processing, object detection, feature extraction, training data, and classification. We found that BPA and its Analogs induced phenotypic 3D structural changes and M phase arrest in a dose-dependent manner. BPAF induced an accumulation of multinucleated cells, which was associated with an increase of DNA damage response, and impairment of cellular actin filaments. Overall, through the implementation of machine learning paired with image-based HCA, we have demonstrated a new and effective means of classifying multiple toxic endpoints in the co-culture model treated with BPA and its Analogs. Therefore, this ML-based HCA approach provided an in-depth analysis of high-content datasets, powered up image-based multivariate data, and allowed rapid and objective high-throughput screening for future environmental toxicity testing.

### Materials and Methods

### Chemicals

Dulbecco's Modified Eagle Medium (DMEM), Modified Eagle's Medium/Nutrient Mixture F-12 (DME/F12), horse serum, and penicillin-streptomycin were purchased from GE Healthcare Life Sciences (Logan, Utah.). Fetal bovine serum (FBS), 4,4′-(propane-2,2-diyl) diphenol (BPA, ≥99%), 4,4′-sulfonyldiphenol (BPS, 98%), 2,2′,6,6′-Tetrabromo-4,4′-isopropylidenediphenol (TBBPA, 97%), and neutral red (NR) were purchased from Sigma-Aldrich (St Louis, Mo.). Nu-Serum was purchased from BD BioScience (Redford, Mass.). 4-[1,1,1,3,3,3-Hexafluoro-2-(4-hydroxyphenyl)propan-2-yl]phenol (BPAF, 98%) was purchased from Alfa Aesar (Ward Hill, Mass.). 5-Bromo-2′-deoxyuridine (BrdU, 99%) was purchased from Thermo Scientific (Waltham, Mass.). 4% Paraformaldehyde was purchased from Boston Bioproducts (Ashland, Mass.).

### Establishment of Testicular Cell Co-Culture Model and Treatment

The testicular cell co-culture model was established as reported in Example 1 Briefly, mouse spermatogonial cell line C18-4 was established via germ cells isolated from the testes of 6 day old Balb/c mice. This cell line was selected as it showed morphological features of type A spermatogonia, expressed testicular germ cell-specific genes, such as GFRA1, Dazl, Ret and stem cell-specific genes such as psiwi12 and prame11 (Hofmann et al., 2005). Mouse Leydig (TM3) and Sertoli (TM4) cell lines were purchased from ATCC, and these cells were isolated from prepubertal mouse gonads. C18-4 cells were maintained in DMEM composed of 5% FBS, and 100 U/ml streptomycin and penicillin in a 33° C., 5% CO2 humidified environment in a sub-confluent condition, and were passaged every 3-4 days TM3 and TM4 cells were cultured in DME/F12 composed of 1.25% FBS, 2.5% horse serum, and 100 U/ml streptomycin and penicillin at 37° C., 5% CO2 in a sub-confluent condition, and were passaged every 2-3 days. When the cells reached 70-80% confluence, a total of 1.5×10⁴ cells per well were inoculated into a 96-well plate. The percentages of spermatogonial, Sertoli, and Leydig cells in the co-culture model were 80%, 15%, and 5%, respectively. The co-cultures were maintained in DMEM composed of 2.5% Nu-serum, and 100 U/ml streptomycin and penicillin in a 33° C., 5% CO2 humidified environment. After, an overnight incubation period, the co-cultures reached 100% confluence and were then treated with various doses of BPA, BPS, BPAF, and TBBPA for the indicated doses and time periods.

### NR Dye Uptake Assay

Cell viability was determined through the Neutral Red uptake assay, which is based on the ability of viable cells to incorporate NR dye into their lysosomes, while the dye is not retained if the cell dies (Repetto et al., 2008). The co-cultures were treated with various doses of BPA, or BPS, (25, 50, 100, 200 and 400 μM), and BPAF or TBBPA (2.5, 5, 10, 25 and 50 μM) for 24, 48, and 72 h. The vehicle controls were set as the co-cultures treated with the vehicle (0.05% DMSO) After the treatments, the culture medium was replaced by fresh medium containing NR (50 μg/ml). After a 3 h incubation period, the co-cultures were washed with phosphate buffered saline (PBS), and the NR dye was eluted with 100 μl of a 0.5% acetic acid/50% ethanol solution. The plate was then gently shaken, and absorbance values were measured at 540 nm with a Synergy HT microplate reader (BioTek, VT). Cell viability was presented as a percentage of the mean of vehicle controls after subtracting background readings.

### Fluorescence Staining and Image Acquisition

To examine DNA synthesis, and conduct cell cycle analysis, the co-cultures were treated with various doses of BPA, or BPS (5, 10, 25, 50 and 100 μM), and BPAF or TBBPA (1, 2.5, 5, 10 and 15 μM) for 24, 48 and 72 h. BrdU incorporation, cell fixation, BrdU, and DNA staining followed the previously published protocol (Liang, et al., 2017b). the determination of DNA damage responses and cytoskeleton analysis was conducted after cell fixation. The cells were then permeabilized by a 0.1% Triton X-100 in PBS, and then incubated with a mouse-anti phosphor-histone H2AX (Ser139) (γ-H2AX) overnight at 4° C. After washing the cells with a BPS/BSA the cells were then incubated with goat anti-mouse Dylight 650, and Hoechst 33342 in a BPS/BSA solution for 90 minutes. Finally, prior to acquiring the images, the cells were stained with Alexa Fluor 488 Phallodin for 30 minutes to stain the F-actin (Liang, et al., 2017)

Multi-channel images were then automatically acquired using an Arrayscan™ VTI HCS reader (Thermo Scientific, MA) with the HCS Studio 2.0 Target Activation BioApplication module. Forty-nine fields per well were acquired at 20× and 40× magnification using a Hamamatsu ROCA-ER digital camera in combination with a 0.63× coupler and Carl Zeiss microscope optics in auto-focus mode. Image smoothing was conducted in order to reduce object fragmentation prior to the primary object identification that takes place in the first channel. Channel one (Ch1) applied the BGRFR 386_23 for Hoechst 33342 that was used for auto-focus, object identification, and segmentation. The border objects were then excluded. Channel two (Ch2) applied applied the BGRFR 549_15, which identified the BrdU staining and BGRFR 485_20 to identify the F-actin staining. BGRFR 650_13 was applied in Channel three (Ch3) and identified the γ-H2AX staining.

### High-Content Image Analysis

Multi-Channel images were analyzed using HCS Studio™2.0 TargetActivation BioApplication. Multiple parameters of nuclei were characterized in HCA, including nuclei number, nuclear area, shape, and total DNA intensity. Nuclear shape measurements included P2A, a ratio of the nuclear perimeter squared to 4it*nucleus area (perimeter²/4π*enuclear area) to evaluate nuclear smoothness, and LWR, a ratio of the nuclear length to width, to measure nuclear roundness. For a fairly round and smooth object, the values for P2A and LWR are around 1.0. Total intensity was defined as total pixel intensity within a cell in the respective channel. The total intensities of BrdU, γ-H2AX, and F-actin of the individual cells were also quantified. With forty-nine 20× images acquired from each well, at least 1,000 cells were analyzed per well, and single-cell based data were extracted for further analysis. The experiments were performed with at least four biological replicates and repeated twice.

### Machine Learning-Powered High Content Analysis

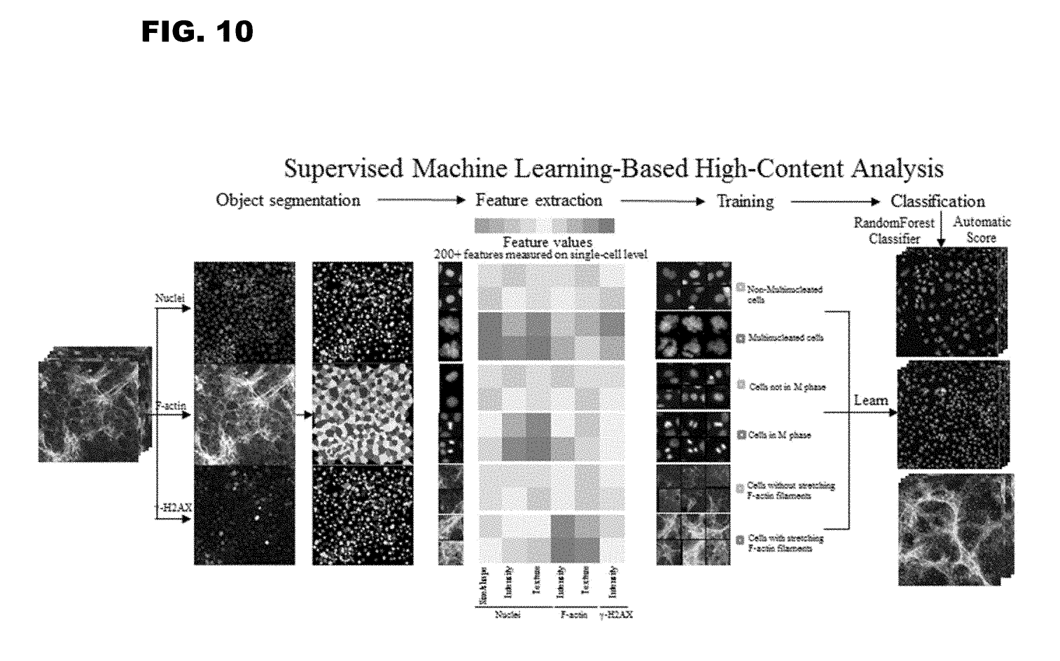

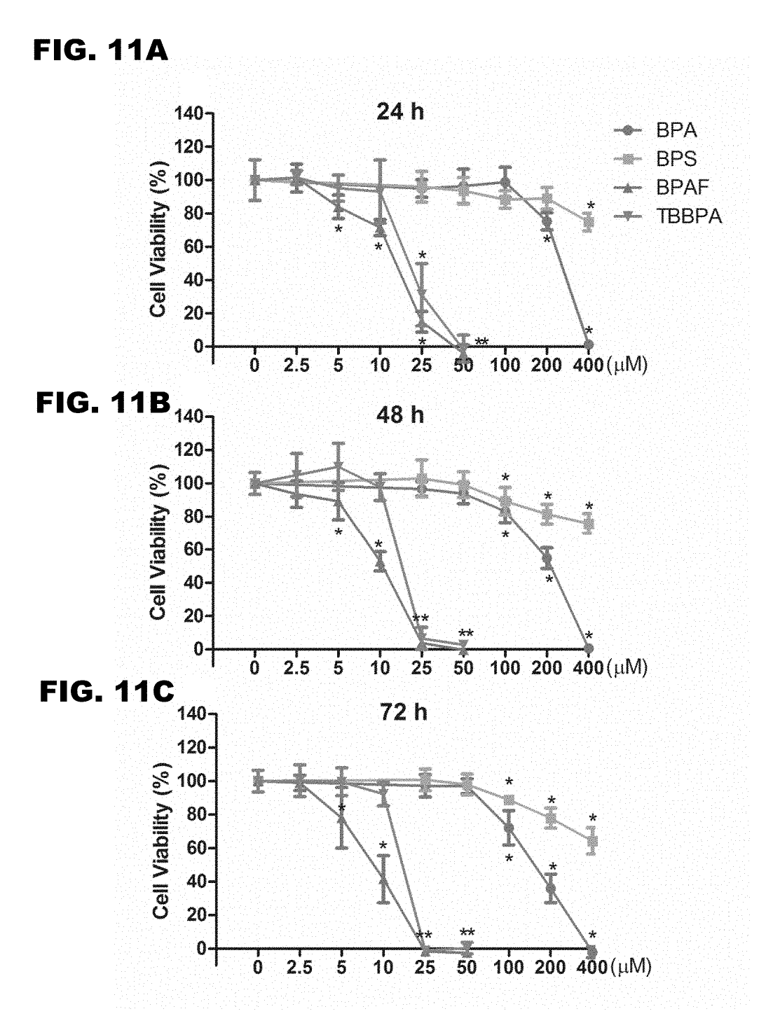

For ML-based HCA as illustrated in FIG. 10, the multi-channel images acquired from the Arrayscan™ VTI HCS reader were analyzed using the open-source software CellProfiler (Broad Institute, MA) (Carpenter et al., 2006). Our processing pipeline included cell segmentation, localization, and measurements of multiple features of single cells. This pipeline identified the nuclei from the Hoechst 33342 (nuclei) channel and used the nuclei as primary objects to assist in the identification of secondary objects, which included F-actin and γ-H2AX in each cell. The pipeline (which is freely available from the authors upon request) has the ability to measure over 200 cellular features, including size, shape, intensity, and texture of nuclei, intensity, and texture of F-actin, and intensity of γ-H2AX in a single cell. In order to measure the nuclear area and shape of a cell, multiple features such as object area, perimeter eccentricity and orientation had to be extracted. Object intensity was determined through measurements of various intensity statistics including the integrated intensity, the mean intensity, and the maximal or minimal pixel intensities within an object or on the object's edge. To quantify the object's texture, Haralick texture features derived from the co-occurrence matrix were employed to calculate the occurrence of pairs of pixels with specific intensity values and spatial relationships in an image. The texture features were described by homogeneity, local variation, randomness, and the contrast of object texture. Supervised ML was performed with the CellProfiler Analyst via the “RandomForest Classifier” algorithm (Broad Institute, MA) (Jones et al., 2008). RandomForest is an ensemble learning method that constructs decision trees with an averaged prediction, and is robust in high-dimensional data analysis with low bias (Breiman, 2001).

In the current study, we quantified three key phenotypic changes in the co-culture model. The first change we noticed was the induction of multinucleated cells, a unique toxicity marker for BPAF observed in spermatogonia cells in the previous study, was once again examined in the current co-culture testicular cell model. As depicted in FIG. 10, the multinucleated phenotype was identified as a cell with giant nuclei and irregular nuclear contour. The second change noticed was through HCA-based cell cycle analysis which revealed that BPAF significantly induced G2/M phase arrest; however, this DNA histogram could not detemine the exact M phase. Mitotic phenotypes were identified as cells with small condensed nuclei with a round shape (prometaphase), condensed nuclei with shallow concavities (metaphase), and nuclei with separated and aligned chromosomes (anaphase, telophase, and late telophase). Lastly, we observed the formation of 3D structure in the co-cultures at 48, 72, and 96 h at 100% confluency, and that the treatment of BPA or its Analogs resulted in the disturbance of these structures. Cells with phenotypic 3D structure have F-actin bundles stretching across their cytoplasm. During the training process, a small set of unclassified objects were manually sorted into two classes, including non-multinucleated cells or multinucleated cells, cells not in M phases or cells in M phase, and cells with or without stretching F-actin filaments. The maximum number of features was set as 20 in order to generate rules for phenotypic classification, and those features were automatically selected by the RandomForest classifier to capture the subtle differences between the classes. The phenotype classification was based on the images acquired by Arrayscan™ VTI HCS reader, and multiple features were measured by CellProfiler. The input features included values describing nuclei size and shape, DNA intensity and texture, F-actin intensity and texture, and γ-H2AX intensity at the single-cell level. For multinucleated cells and M phase cells, the training set was established by visual examination of the images in the nuclei channel. For a cell with stretching F-actin filaments, initial manual classification during the training process was based on multi-channel images, including nuclei, F-actin and γ-H2AX. The results were presented in terms of the number of cells in predefined classes and total cell number per image field. The field-based data were averaged for the well-based condition.

### Statistical Analysis

The data obtained from the HCS Studio·8 2.0 TargetActivation BioApplication, CellProfiler and CellProfiler Analyst were exported and further analyzed using the JMP statistical analysis package (SAS Institute, NC). To remove cell clumps, nuclei with areas larger than 1000 μM² were excluded. For each plate, the vehicle control showed consistent measurements for all endpoints tested. For intra-plate normalization, data were normalized to the overall scaling factors, which was the mean of medians of the vehicle controls in each plate. The single cell-based data were averaged for the well-based condition. BrdU-positive cells were set by the total intensity of BrdU in the control at over 25,000 pixels. γ-H2AX positive cells were set by the total intensity of γ-H2AX in the control at over 120,000 pixels. The median lethal concentrations (LC50) were calculated using a nonlinear regression curve fit on GraphPad Prism 5 (San Diego, Calif.). To examine the correlation between the cytoskeleton and DNA damage responses in multinucleated cells, the Spearman correlation analysis was conducted between the total intensity of F-actin and γ-H2AX for 24, 48, and 72 h using cell-based data. Data were presented as mean±standard deviation (SD). Statistical significance was determined using one-way ANOVA followed by Tukey-Kramer all pairs comparison. The p-value less than 0.05 denoted a significant difference compared to the vehicle control (*).

### Computational Resources

Both image processing workflows were tested on a desktop Windows 7 workstation (16 GB RAM) and CellProfiler pipelines were submitted for processing on a Linux cluster. Software versions used were CellProfiler version 2.1.2 v2015_08_05, CellProfiler Analyst 2.0 v2014_04_01, and ilastik version 0.5.12; links to Windows binary versions of these are available on the world wide web at cellprofile.org/published_pipelines.shtml.

### Results

### BPA and its Analogs Induced Time and Dose-Dependent Cytotoxicity in the Testicular Cell Co-Culture Model.

To determine the appropriate concentrations of BPA and its Analogs to be used in the HCA experiments, cell viability was measured by the NR uptake assay. FIG. 11 shows dose- and time-dependent decreases in cell viability in the co-culture models treated with BPA or its Analogs for 24 (A), 48 (B) and 72 h (C). BPA and BPS treatments significantly decreased cell viability starting at doses of 200 and 400 μM, respectively, for 24 h, and 100 μM for 48 and 72 h. BPAF and TBBPA significantly reduced cell viability starting at concentrations of 5 and 25 μM, respectively, for 24, 48, and 72 h. The LC50 values for 72 h time period were 8.5, 16.8, 150.2, and 625.8 μM for BPAF, TBBPA, BPA, and BPS, respectively. In the following HCA experiments, 100 μM was selected as the highest concentration dose for BPA and BPS treatments, and 15 μM for BPAF and TBBPA treatments.

### BPA and its Analogs Altered Nuclear Morphology and Cell Number in the Testicular Cell Co-Culture Model.

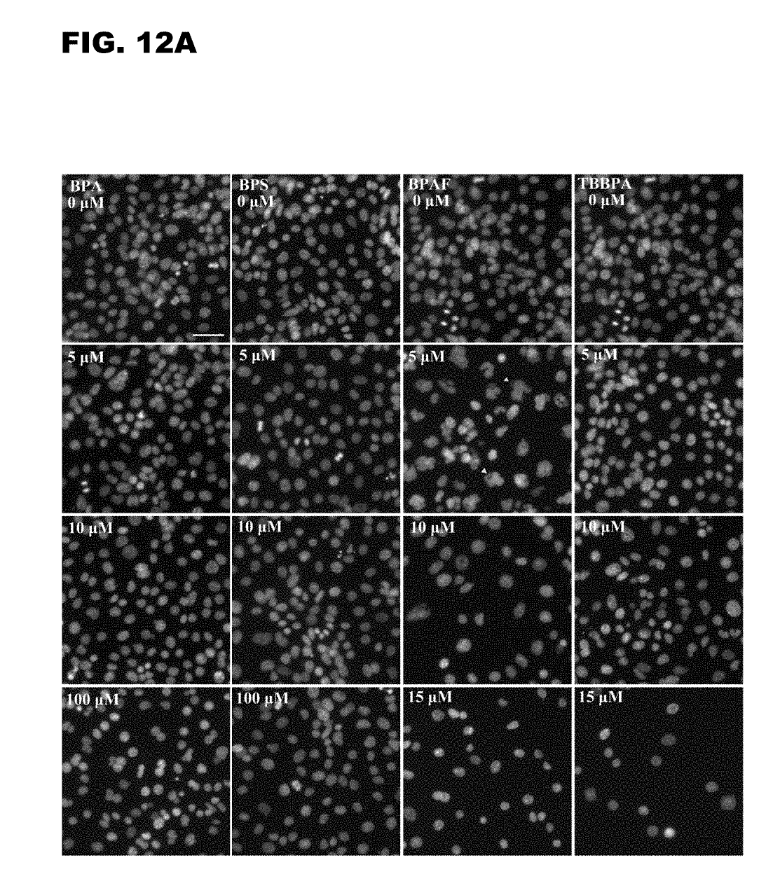

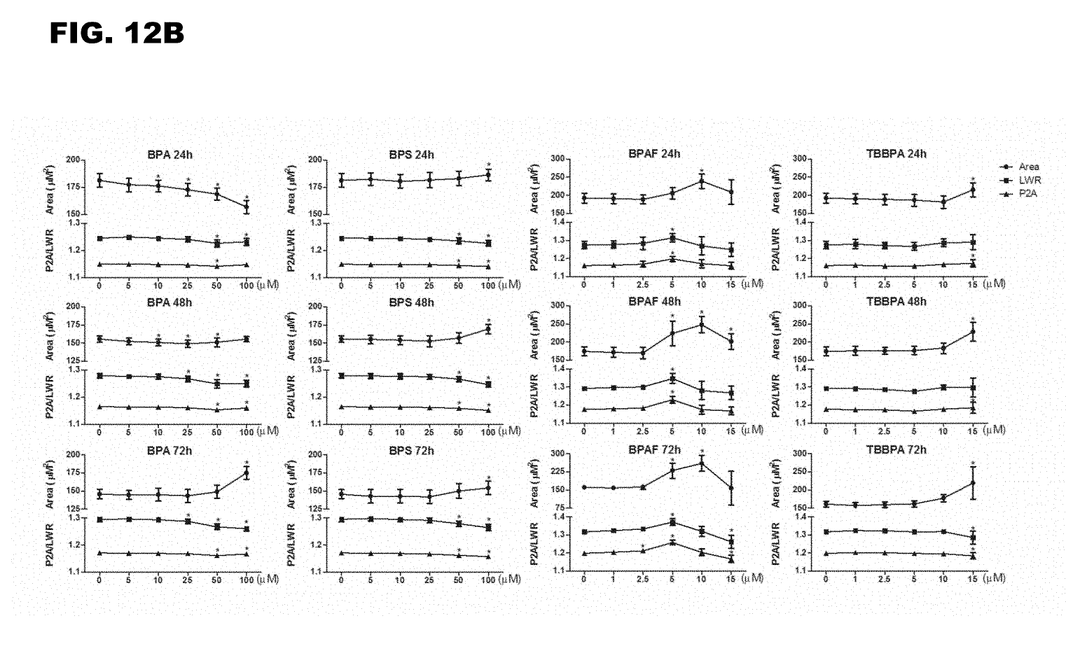

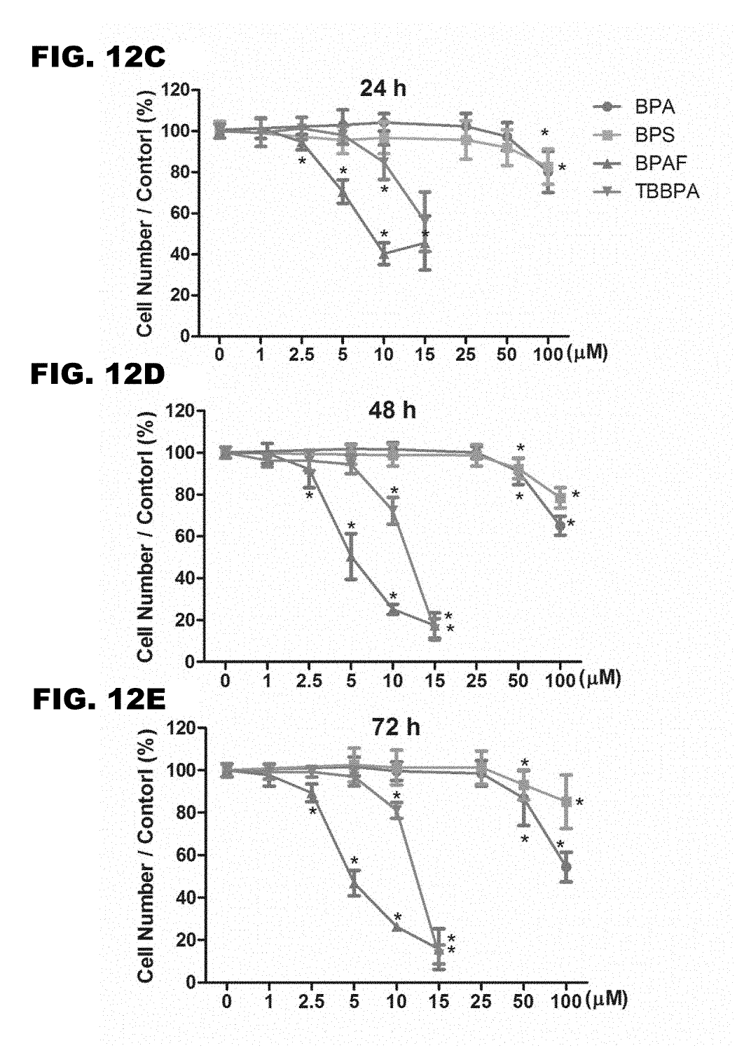

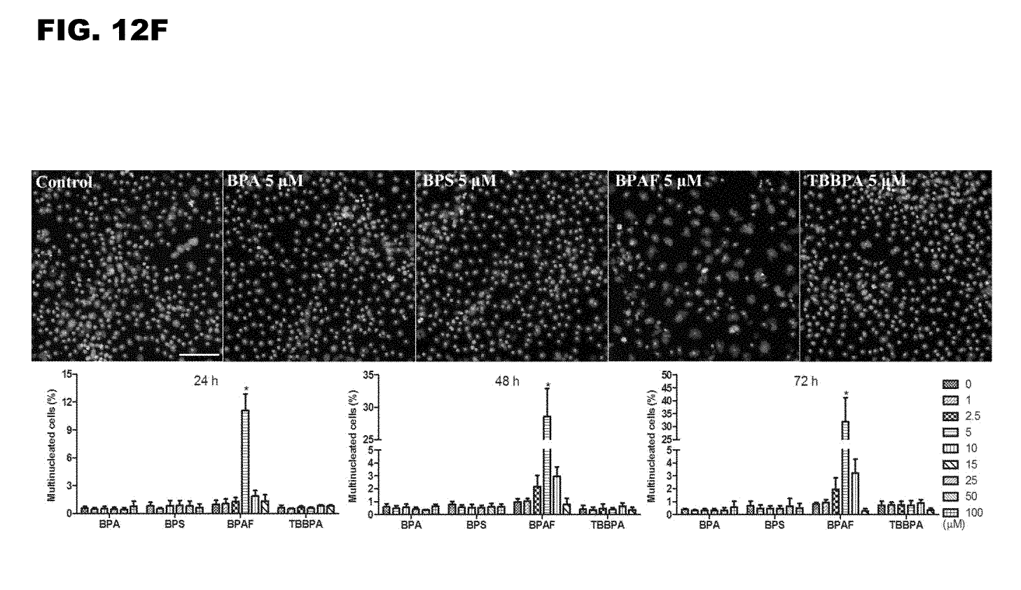

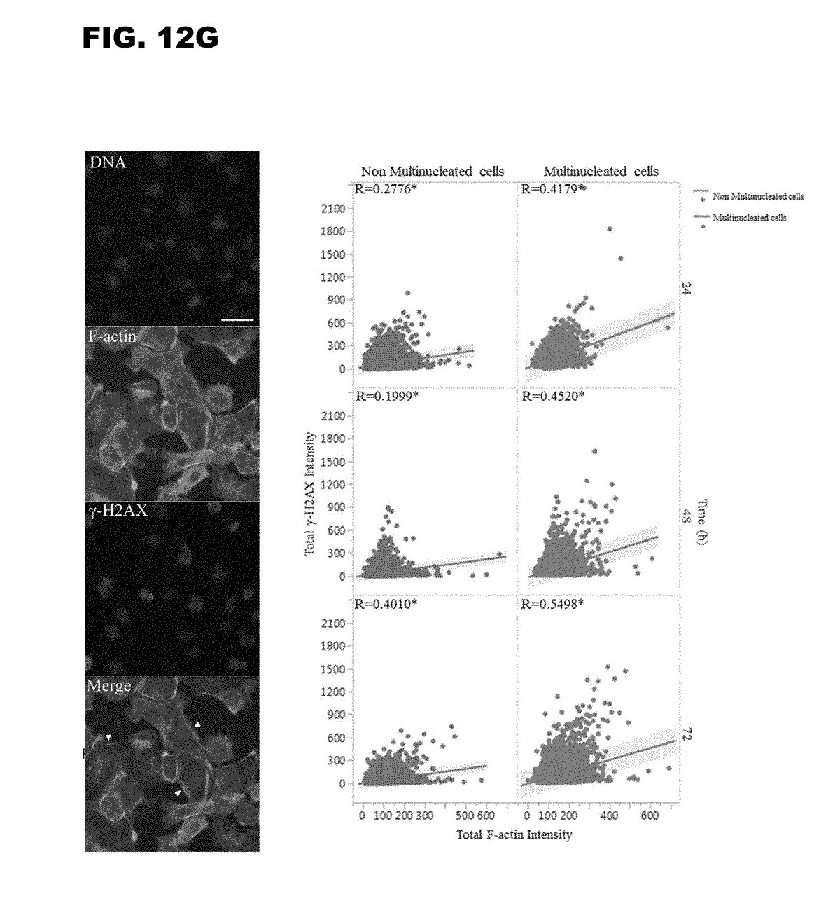

Nuclear morphology is considered to be a sensitive endpoint for detecting chemical toxicity in HCA assays (Martin et al., 2014; O'Brien et al., 2006). In our HCA, we measured multiple morphological parameters of nuclei, including nuclear area, roundness (LWR) and smoothness (P2A). FIG. 12A shows representative images of nuclear morphology after 48 h with or without treatments. Notable decreases in cell number were observed in all four chemical treatments. Additionally, multinucleation (denoted by the arrow) was observed in BPAF treatments at a dose of 5 μM (FIG. 11A). As shown in FIG. 11B, nuclear morphology was quantified in the co-cultures treated with BPA and its Analogs after 24, 48, and 72 h. Significant increases in nuclear area were observed in the co-cultures treated with BPAF at a dose of 10 μM for 24 h, 5 to 15 μM for 48 h, and 5 and 10 μM for 72 h; TBBPA at a dose of 15 μM for 24, 48, and 72 h; BPA at 100 μM for 24, 48 and 72 h; and BPS at a dose of 100 μM for 72 h. Significant decreases in nuclear area were observed in the co-cultures treated with BPA starting at 10 μM for 24 h, and 10 to 50 μM for 48 h. BPA treatment significantly reduced LWR starting at 50 μM for 24 h, 25 μM for 48 and 72 h, and reduced P2A at a dose of 50 μM for 24 h, and 50 and 100 μM for 48 and 72 h. BPS treatment significantly decreased LWR and P2A at doses of 50 and 100 μM for 24, 48, and 72 h. BPAF significantly increased LWR and P2A at a dose of 5 μM for 24 and 48 h, 2.5 and 5 μM for 72 h, and decreased LWR and P2A at a dose of 15 μM for 72 h. TBBPA treatment significantly increased P2A at a dose of 15 μM for 24 and 48 h, and decreased LWR and P2A at doses of 15 μM for 72 h. The differential nuclear area alterations in 50 and 100 μM BPA treatments might reflect 3D structure loss at 100 μM. The differential nuclear morphological changes of BPAF treatments at 5 and 15 μM could be explained by early adaptive response to low-dose treatment and loss of cellular homeostasis at high-dose treatment.

In FIG. 12C-E, BPA and BPS treatments significantly reduced cell number at a dose of 100 μM for 24 h, and at doses of 50 and100 μM for 48 and 72 h. BPAF treatment decreased cell number starting at 2.5 μM for 24, 48, and 72 h, while TBBPA reduced cell number starting at 10 μM for 24, 48, and 72 h. This data indicated that cell number counting in the HCA assay is more sensitive than the traditional NR uptake cytotoxicity assays.

To quantify the multinucleated cells in the co-cultures treated with the various compounds, we applied a supervised ML-based HCA of imaging data. In FIG. 12F, BPAF treatment significantly induced multinucleated cells at a dose of 5 μM for 24, 48, and 72 h, whereas BPA, BPS, and TBBPA treatments did not induce multinucleation in cells. In order to elucidate how multinucleation related to other pathological features, we applied a supervised ML, and classified the HCA imaging dataset into multinucleated and non-multinucleated cells. With the utilization of a Spearman correlation analysis, we aimed to decipher the association of multinucleation with DNA damage responses and F-actin. FIG. 12G, shows the higher resulting positive correlation between total F-actin and γ-H2AX intensity observed in the multinucleated cells, as compared to the intensities in the non-multinucleated cells with BPAF treatment at a dose of 5 μM for 24, 48, and 72 h. Thus, this data suggests that cytoskeleton perturbations might co-occur with DNA damage in the multinucleated cells.

### BPA and its Analogs Perturbed DNA Synthesis and Induced M Phase Arrest in the Testicular Cell Co-Culture Model.

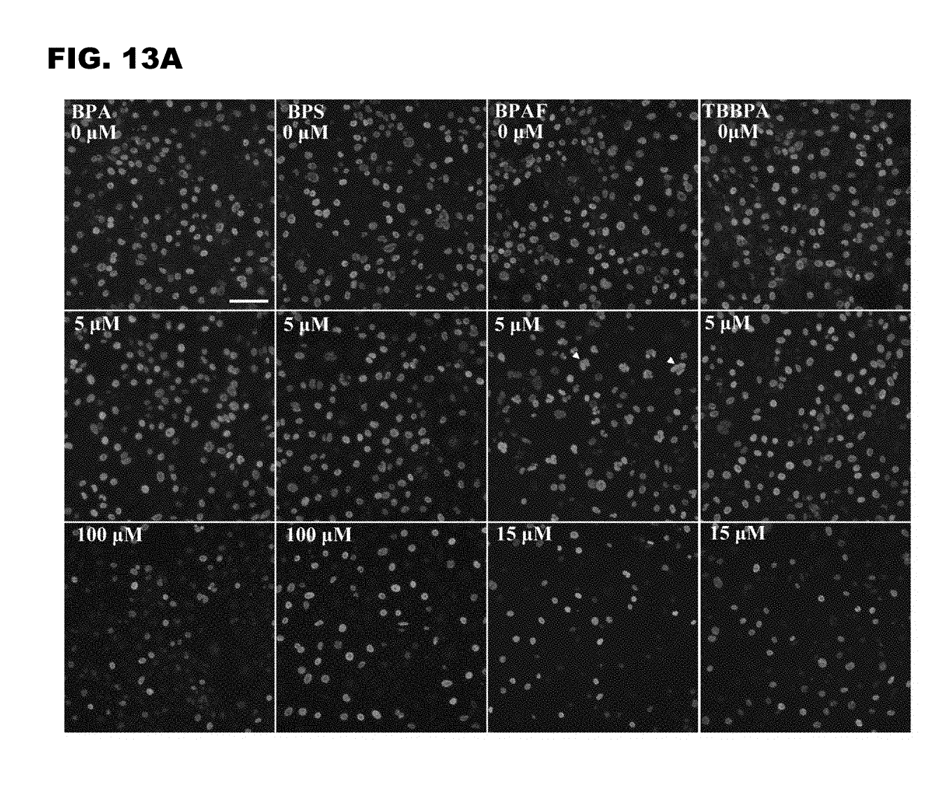

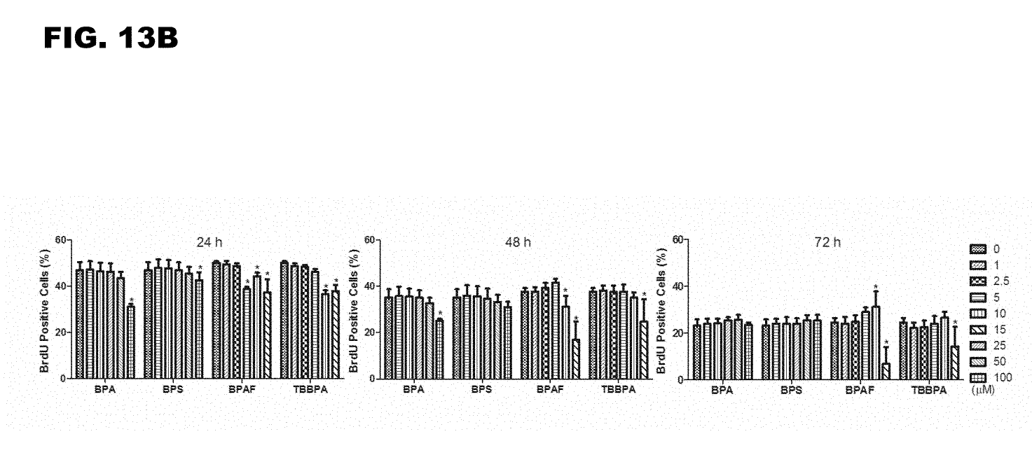

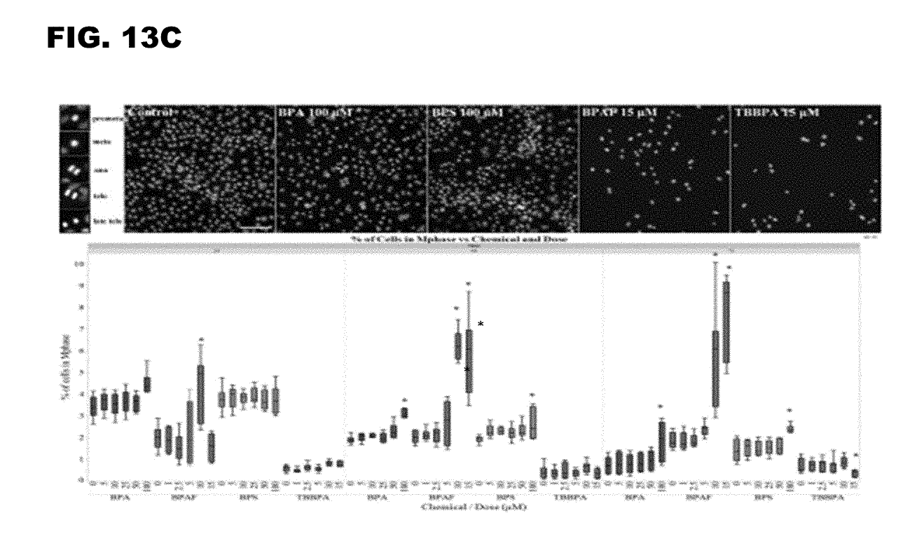

Cell cycle progression is essential for germ cell renewal and progeny cell production (De Rooij and Russell, 2000). We previously developed a HCA assay to measure DNA synthesis and cell cycle progression. FIG. 13A shows representative images of BrdU incorporation. Notable decreases in BrdU-positive cells were observed in BPA, BPS, BPAF and TBBPA treatments after the 24 h time point. In BPAF treatment, multinucleated cells exhibited BrdU-positive staining at a dose of 5 μM (arrow). Significant decreases in BrdU-positive cells were observed in the co-cultures treated with BPA at a dose of 100 μM for 24 and 48 h, BPS at a dose of 100 μM for 24 h, BPAF at doses of 5, 10, and 15 μM for 24 h, 10 and 15 μM for 48, and 15 μM for 72 h, and TBBPA at doses of 10 and 15 μM at 24 h, and 15 μM for 48 and 72 h, which suggested DNA synthesis inhibition due to these chemical treatments. BPAF treatment induced BrdU-positive cells at a dose of 10 μM for 72 h (FIG. 13B). BPAF treatment at a dose of 10 μM inhibited DNA synthesis at first, but interestingly promoted it at longer exposure periods, suggesting that BPAF could potentially induce abnormal DNA synthesis at later time points.

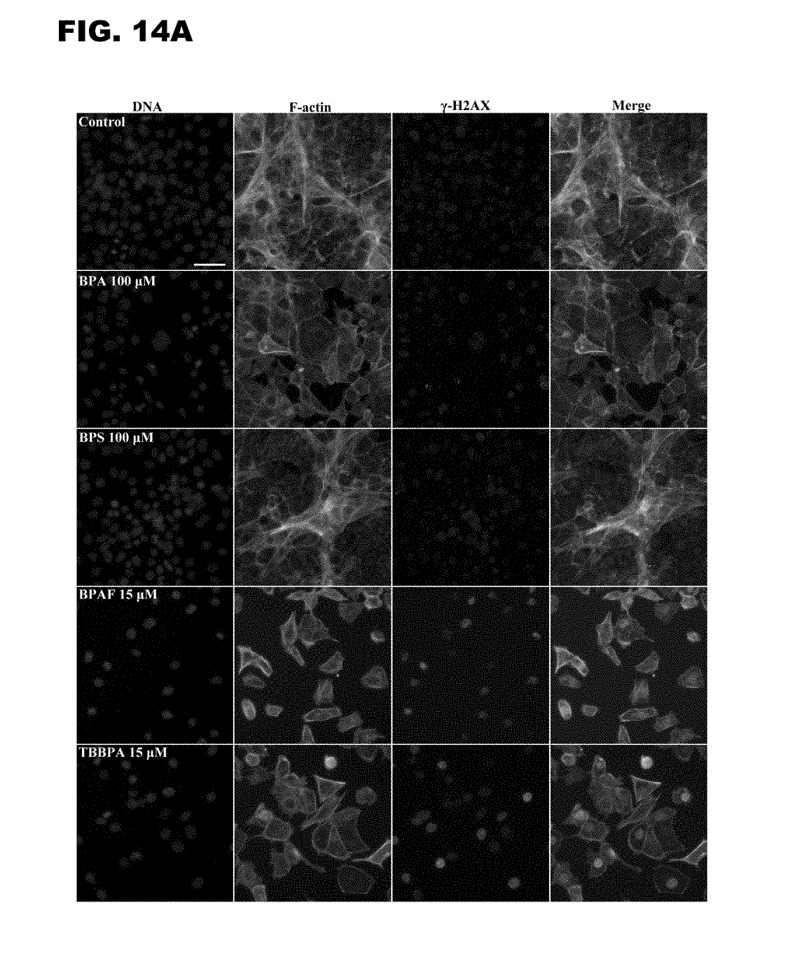

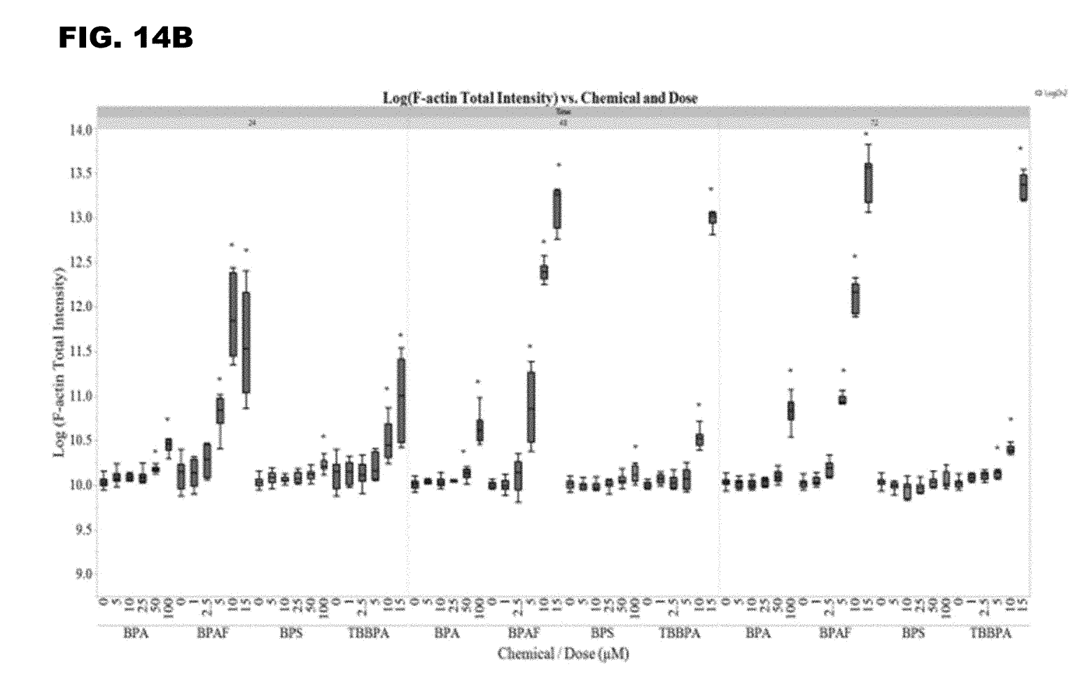

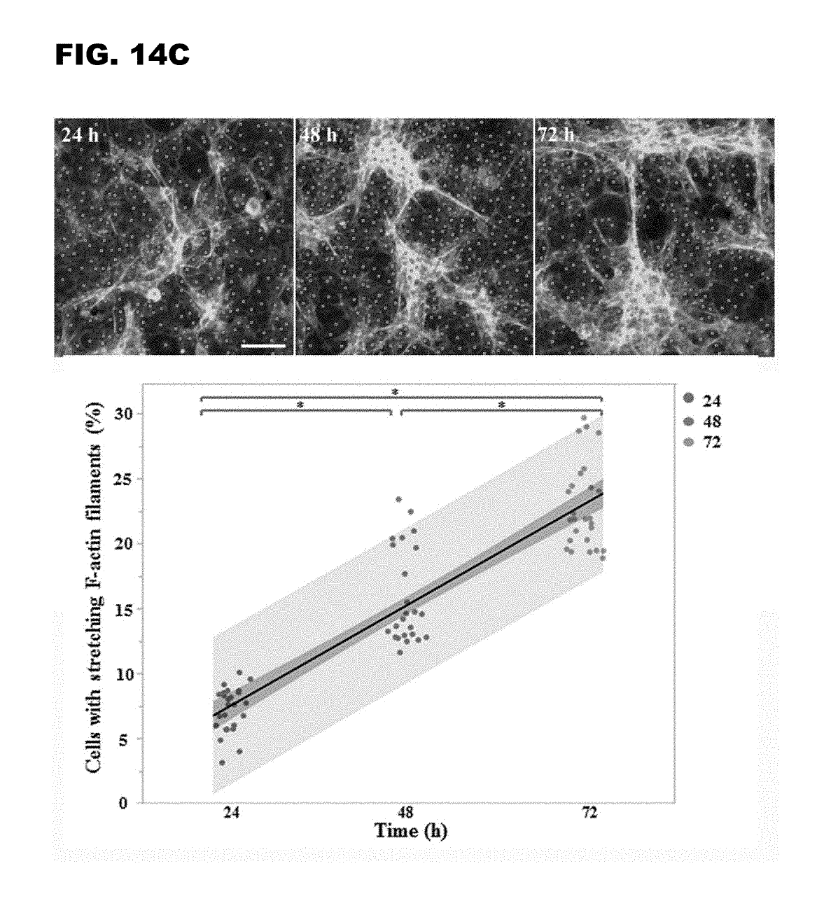

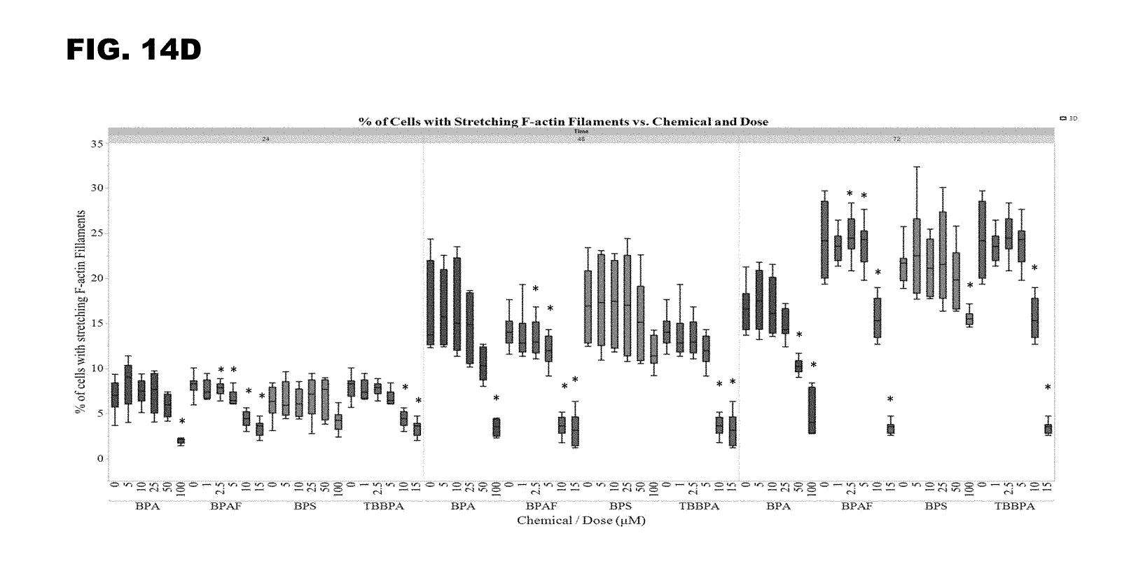

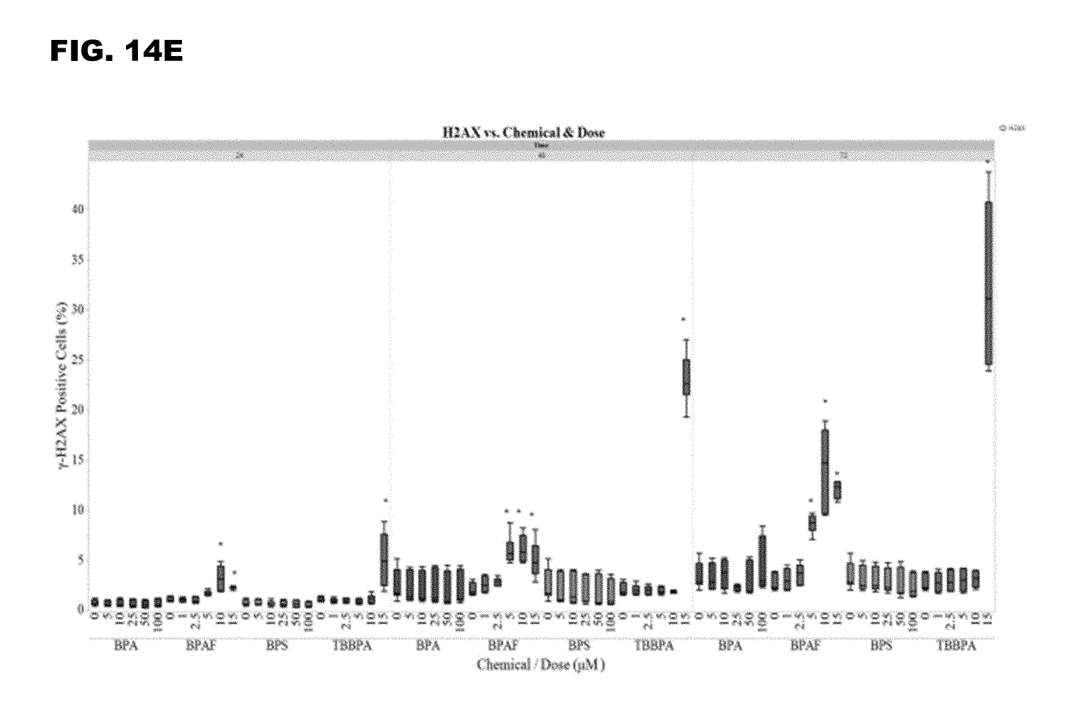

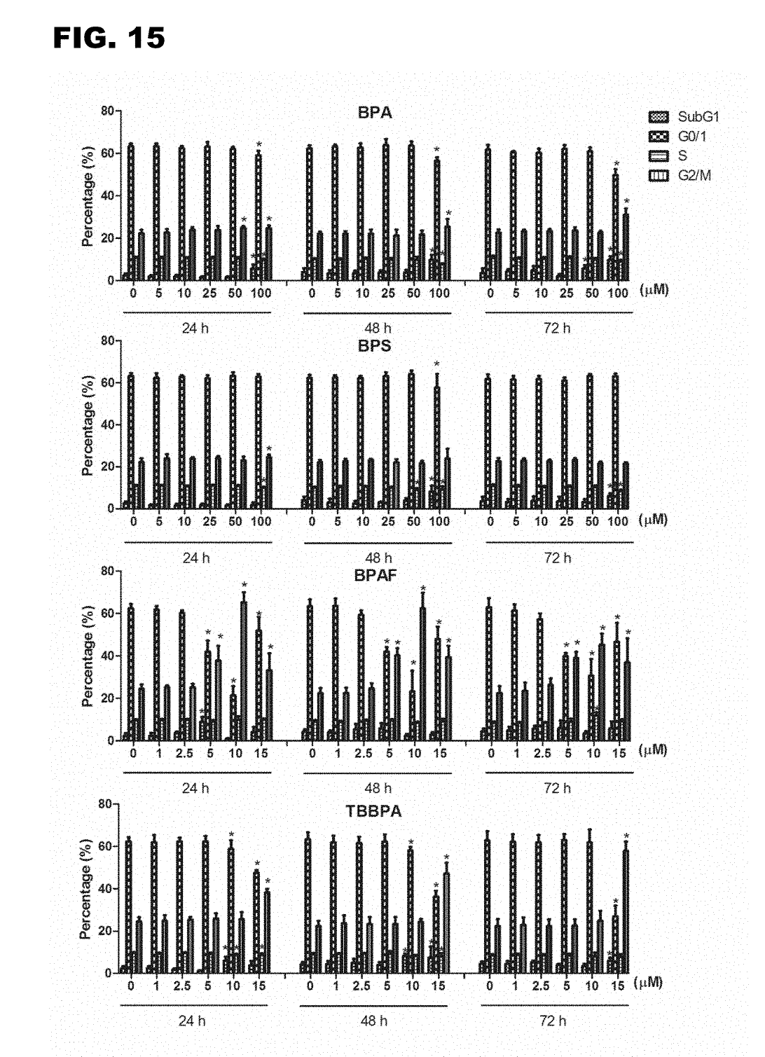

In addition, we observed that BPA and its Analogs preturbed cell cycle progression in the co-culture modelin a dose-and time-dependent manner (FIG. 14). To identify the effects of BPA and its Analogs on mitosis progression, a supervised ML approach was conducted and various phenotypic features of M phase were extracted. The various mitotic phenotypes included:nuclei in pro-metaphase (pro meta), metaphase (meta), anaphase (ana), telophase (telo) and late-telophase (late-telo) which were observed in the co-cultures (FIG. 13C). Significant inductions of cells in M phase were observed in the co-cultures treated with BPA and BPS at a dose of 100 μM for 48 and 72 h; BPAF at a dose of 10 μM for 24 h, and at doses of 10 and 15 μM for 48 and 72 h; and TBBPA at a dose of 15 μM for 24, 48, and 72 h. The results suggested that BPA and its Analogs induced M-phase arrest in the co-cultures.

### BPA and its Analogs Perturbed F-Actin Cytoskeleton, Induced Phenotypic 3D Structure Changes, and DNA Damage Responses in the Testicular Cell Co-Culture Model.

F-actin structures are involved in various cellular processes including germ cell nuclei remodeling, cytoplasm reduction, and cell movement during spermatogenesis (Niedenberger et al., 2013). In Sertoli cells, parallel actin bundles form ectoplasmic specialization (ES) to provide an immunological barrier for germ cells and regulate elongated spermatid orientation and spermatozoa release (Cheng and Mruk, 2002; Setchell, 2008; Wong et al., 2008). As shown in FIG. 14A, F-actin filaments in the untreated co-cultures exhibited two typical patterns, cortical actin filaments adjacent to the cell edge and thick stress fiber bundles through the cytosol. The cells with stretching F-actin bundles in the cytoplasm further formed unique cord-like structures in the co-cultures. BPA, BPAF, and TBBPA treatments dramatically perturbed these structures and induced gel-like networks of cross-branched actin filaments, whereas BPS treatment induced no notable changes. FIG. 14B shows the quantification of the log-transformed total intensity of F-actin. Significant increases in F-actin total intensity were observed in the co-cultures treated with 50 and 100 μM BPA for 24 and 48 h; 100 μM BPA for 72 h; 100 μM BPS for 24 and 48 h; 5, 10 and 15 μM BPAF for 24, 48 and 72 h; 10 and 15 μM TBBPA for 24 and 48 h, and 5 to 15 μM TBBPA for 72 h.

In order to quantify phenotypic 3D structure in the co-cultures and the cells' response to chemical perturbations, the cells with F-actin bundles stretching across the cytosol were quantified based on a supervised ML approach. FIG. 14C shows time-dependent increases of cellular phenotypic 3D structure in the controls for 24, 48, and 72 h, indicating the formation of 3D structure in the co-cultures. As shown in FIG. 14D, significance decreases of this phenotype were observed in the co-cultures treated with BPA at a dose of 100 μM for 24 h; 50 and 100 μM for 48 and 72 h; BPS at a dose of 100 μM for 72 h; BPAF at doses of 2.5, 5, 10 and 15 μM for 24 and 48 h, and 5, 10 and 15 μM for 72 h; and TBBPA at doses of 10 and 15 μM for 24, 48, and 72 h.

### Discussion:

Although current HCA has emerged as a powerful tool for analyzing multiparametric data for toxicity profiling, data exploration lags behind. High-content analysis has focused on one or two image-related features and has generated population-averaged readouts that simply reflect alterations of morphological features in each sample (Singh et al., 2014). In addition, the aggregation of single cell-level data usually masks phenotypic heterogeneity within cells, especially when a phenotypic change only occurred in a small specific subpopulation (Altschuler and Wu, 2010). Some phenotypic changes could be quantified by specific fluorescent labeling; however, it is difficult to evaluate multiple parameters precisely due to the multiplex fluorescent labels used within a single assay (Heynen-Genel et al., 2012). Therefore, it is essential to employ an advanced computational approach to integrate multi-dimensional HCA data on the single-cell level to precisely quantify the complex phenotypes of interest. ML that selects and integrates multiple features for automated phenotypic classification has been used to score various phenotypes in an unbiased manner (Bakal et al., 2007; Jones et al., 2009; Loo et al., 2007; Neumann et al., 2006). The classification algorithm was generated from a training dataset by manual annotation of some representative images according to the predefined classes. After the training period, the ML algorithm could automatically discriminate among the classes in the full dataset. This supervised ML approach reduced the workload that comes with the manual adaption of parameter sets, and increased objectivity, consistency, and accuracy in large-scale data sets (Sommer and Gerlich, 2013; Tarca et al., 2007). In recent years, ML combined with HCA has been used to generate phenotypic profiling in various research fields (Conrad and Gerlich, 2010; Fuchs et al., 2010; Fuller et al., 2016; Leonard et al., 2015; Mata et al., 2016; Schmitz et al., 2010).

Sertoli and Leydig cells play critical roles in maintaining spermatogenesis and reproductive functions by providing physiological and nutritional support for germ cell mitosis, meiosis and movement. These two cell types have been employed in various co-culture systems to improve the physical relevance of in vitro models, and examine the reproductive toxicities of various chemicals. Recently, our lab combined the C18-4 spermatogonial cell line, the TM3 Leydig cell line, and the TM4 Sertoli cell line to construct an animal-free testicular cell co-culture model that mimics the in vivo testicular structure. Leydig cells, TM3, and Sertoli cells, TM4, were previously established from the testis of immature BALB/c mice and exhibited distinct morphology and growth response to hormones (Mather, 1980). The incorporation of these testicular somatic cells in this co-culture model showed a distinct cord-like structure, high specificity, and high sensitivity in the classification of reproductive toxicants (Example 1).

Nuclear morphological features have been suggested as useful indicators in various adverse cellular events (Eidet et al., 2014; Ikeguchi et al., 1999). In HCA assays, the quantitative assessment of multiple nuclear parameters have been demonstrated as sensitive markers for detecting early cytotoxic effects. In the present study, the quantification of nuclear morphology revealed that BPA treatment significantly altered nuclear area at a non-lethal dose, which is consistent with previous in vivo studies in which exposure to BPA induced abnormal nuclear morphology in rat mammary glands and mice testes (Ibrahim et al., 2016; Takao et al., 1999). In addition, the significant changes of P2A and LWR in the cells treated with BPA and BPS could be detected at lower doses in the co-cultures, when compared to the single cell cultures. These data suggested that the alteration of nuclear morphology in the co-cultures could be a sensitive endpoint in the detection of chemical toxicity.

In this study, we developed a ML-based phenotypic classification based on multiple subcellular features extracted from nuclear morphology, the texture and intensity of targeted proteins. These results revealed that BPAF induced dose- and time-dependent multinucleated cells in the co-cultures. The induction of multinucleated gonocytes has been reported as a reproductive toxicity marker in animal models and in humans in response to environmental chemicals, including di-(n-butyl) phthalate (DBP), BPA, andrographolide, and aflatoxin (Akbarsha and Murugaian, 2000; Barlow et al., 2004; Faridha et al., 2007; Gallegos-Avila et al., 2010; Mylchreest et al., 2002; Takao, et al., 1999). Although the underlying mechanism of multinucleated cell formation is still unclear, Faridha et al. reported that the generation of multinucleated spermatids was through the opening of the cytoplasmic bridge and merging of multiple cells (Faridha, et al., 2007). In addition, the overexpression of p190RhoGAP, an actin stress fiber regulator, has been associated with multinucleated phenotypes (Su et al., 2003). Furthermore, the perturbation of the cytoskeleton texture co-occurred with the formation of multinucleated cells, which could be explained by the alteration of compressive forces driven by perinuclear actin networks (Chen et al., 2015). Compared to non-multinucleated cells in the same treatment conditions, multinucleated cells exhibited higher correlations between cytoskeleton perturbation and DNA damage responses on the single-cell level, showing the unique biological characteristics of these cells. It has been reported that DNA damage induced dramatic alterations to nuclear and cytoplasmic actin, and F-actin polymerization served as a negative modulator in DNA damage responses (Belin et al., 2015; Chang et al., 2015; Wang et al., 2013; Zuchero et al., 2012). In future studies, the underlying mechanism of BPAF-induced multinucleation of testicular cells should be examined.

M phase, one of the most important events for successful cell reproduction, in which replicated chromosomes were segregated into two daughter cells (Nurse, 1990). The identification and quantification of cell populations in M phase usually requires additional staining using mitotic specific markers, such as anti-phosphorylated (ser10) H3 (Lyman et al., 2011). In one of our lab's previous studies, we developed an HCA approach to generate a cell cycle profile with discrete SubG1, G0/1, S and G2/M phases in the spermatogonial cell line (Liang, et al., 2017b). However, this profiler could not provide a quantification of cell population in M phase. Blasi et. al recently reported a label-free quantification of mitotic cell cycle phase by applying supervised ML to multi-dimensional features of single cells (Blasi et al., 2016). Our lab has since established a ML analysis pipeline in the co-cultures, which was able to recognize and quantify the cells in M phase based on morphological, texture and intensity features extracted from the multi-channel fluorescence staining. We have shown M phase arrest in the co-cultures treated with BPA and its Analogs in a dose- and time-dependent manner, illustrating the chemical specific effect on cell cycle progression. The results presented here are consistent with previous findings showing BPA exposure significantly perturbed spermatogenesis in animal models and inhibited cell proliferation in Sertoli cell TM4 and Leydig cell TM3 (Ali et al., 2014; Chen et al., 2016b; Liu et al., 2013; Pereira et al., 2014).

Actin, one of major component of the cytoskeleton, has been shown to play essential roles in cell movement, cargo transportation, acrosome reaction, and nuclear modification during spermatogenesis (Kierszenbaum and Tres, 2004; Sun et al., 2011). Alteration of F-actin intensity is a sensitive indicator for monitoring the adverse effects of environmental exposure. However, the quantification of F-actin total intensity might not reflect spatial alterations of F-actin filaments in the cytoplasm of cells. In the co-culture model, we observed two types of F-actin fibers, which included the dense cortex F-actin on the cell edge and the F-actin filaments stretching across the cytosol that assembled forming the 3D structure. When an ML approach was applied to recognize and quantify the cells with stretching F-actin bundles across cytosol, it demonstrated significant decreases of this specific phenotype in the co-cultures treated with different chemicals. In the previous study, a combination of cell-type specific markers and HCA cytoskeleton analysis revealed that Sertoli cells exhibited stretching F-actin bundles in the co-cultures (Example 1). Within the Sertoli cells, parallel actin bundles formed ectoplasmic specialization, which participates in spermatid head formation, cell movement, elongated spermatid orientation, and spermatozoa release. Damage to Sertoli cells has often resulted in germ cell degeneration and loss (Vidal and Whitney, 2014). Thus, the alteration of cells with stretching F-actin filaments suggested a potential loss of Sertoli cells and perturbation of 3D structure in the co-cultures. Findings supported the concept that if Sertoli cells are damaged then the blood-testis barrier which is crucial in the maintenance and initiation of spermatogenesis after toxicant-induced spermatogenesis, during the stages of spermatogonial differentiation. If the exposure to the toxicant is long enough this can permanently affect the morphology of F-actin in the Sertoli cells leading to infertility. These observations were consistent with previous in vivo studies that BPA or its Analogs treatment altered seminiferous tubule morphology in animal models.

γ-H2AX has been considered a highly specific and sensitive cellular marker for monitoring initiation of DNA damage (Ando et al., 2014; Fu et al., 2012; Garcia-Canton et al., 2013). Studies have shown that exposure to BPA or BPS induced DNA damage response in germ cells (Chen et al., 2016a; Liang et al., 2017a; Liu et al., 2014). In addition, the genotoxicity of BPA and its selected Analogs have been detected in multiple cell lines and followed a cell-type-specific and chemical-specific manner. In the co-cultures, we observed that BPA and BPS treatment did not induce γ-H2AX expression. One of the possible explanations of inconsistency between the co-culture and previous single cell type cultures could be due to differential biotransformation of BPA and its Analogs, and differing DNA damage repair capacity among the cell models. In human breast cancer cells, BPA was able to induce γ-H2AX at a low dose of 10 nM, but in HepG2 cell line, BPA was not able to induce γ-H2AX even at a dose of 100 μM, but bisphenol F was (Audebert et al., 2011; Pfeifer et al., 2015). For the other two compounds tested in the co-culture model, BPAF treatment significantly induced DNA damage response starting at 5 μM, and TBBPA induced a higher degree of DNA damage response at a dose of 15 μM, suggesting higher genotoxicity compared to BPA.

We observed a similar toxicity ranking of BPA and its selected Analogs in the co-culture model as compared to the single spermatogonial cell cultures. BPAF exerted the highest toxicity, followed by TBBPA, BPA, and BPS. This in vitro finding was further supported by an in vivo study that demonstrated that BPAF exposure uniquely impaired pregnancies and sexual development in rats at doses of ˜80 and ˜280 mg/kg, whereas BPA exposure did not alter these reproductive endpoints at similar levels (Sutherland. et al., 2017). Given the similarity of these two chemicals' in vitro estrogen and androgen receptor (ER, AR) activities, the differential reproductive toxicity both in vitro and in vivo potentially suggested that BPAF might partially exert its adverse effects on the reproductive system in an estrogen-independent manner. In addition, recent data has demonstrated that TBBPA did not interact with ER α/β or AR in a panel of in vitro bioassays, but it showed higher toxicity in both testicular cell models, when compared to BPA (Molina-Molina et al., 2013). Future studies will be critical to validate current findings and elucidate the mechanisms of action.

The ML-based HCA for the testicular cell co-culture model still has several limitations and several advantages. Although current supervised ML recognize certain phenotypes of interest based on the predefined knowledge, it still requires the creation of a training set spanning all replicates to improve accuracy. This is a time and labor extensive task, as it involves the manual sorting of images for each parameter examined and fluorescent label used. For the initial training batches, the individual sorting the images should be highly knowledgeable in the histopathology of the cells being examined. Another potential hang up on the ML-based HCA assay could be the computing power needed to efficiently operate this software. The amount of data that HCA produces can slow down most computers without the proper hardware. This ML-based HCA pipeline could take up to three hours per data set on a standard desktop computer. The better the computing power the less time that the extensive classification system will take to perform its tasks. This specific pipeline is set for the parameters examined in this study, if one wanted they would easily be able to manually adjust these parameters for a pipeline and if needed re-train their data set. The individual would then be able to examine the type of cells and parameters that were selected for their study. This in vitro model mimicked a functional in vivo physiology, through including three cell types, this model allowed for the creating of the BTB and also specialized niche's for spermatogonial cell differentiation, allowing our lab to establish more relevant data regarding toxicity to a human cell based model, rather than just one type of cell culture. This will allow for our lab to analyze the differential biotransformation of BPA and its Analogs and an understanding of how these metabolites can affect the different cell types. Another advantage portrayed by this HCA model is that future studies conducted regarding similar parameters examined, that the training set is already created and has shown with high sensitivity and specificity to be an accurate classifier of male reproductive toxicants. This pipeline may be able to help with a quicker and more efficient way of examining and classifying chemical toxicant's and may allow for regulatory decisions to be based off these findings.

In summary, we developed a ML-based HCA approach to characterize the testicular toxicity in a testicular cell co-culture model. Through the utilization of ML, our lab explored the added value of HCA to classify multiple cellular phenotypes and characterize the compound specific testicular toxicity of BPA and its selected Analogs. By integrating machine-learning based approaches with established HCA algorithms, it will be possible to uncover multi-dimensional data and quantify these phenotypic changes in large-scale exposure to environmental chemicals.

### REFERENCES

Akbarsha, M. A., and Murugaian, P. (2000). Aspects of the male reproductive toxicity/male antifertility property of andrographolide in albino rats: effect on the testis and the cauda epididymidal spermatozoa. Phytother Res 14(6), 432-5.

Ali, S., Steinmetz, G., Montillet, G., Perrard, M. H., Loundou, A., Durand, P., Guichaoua, M. R., and Prat, O. (2014). Exposure to low-dose bisphenol A impairs meiosis in the rat seminiferous tubule culture model: a physiotoxicogenomic approach. Plos One 9(9), e106245.

Altschuler, S. J., and Wu, L. F. (2010). Cellular Heterogeneity: Do Differences Make a Difference? Cell 141(4), 559-563.

Ando, M., Yoshikawa, K., Iwase, Y., and Ishiura, S. (2014). Usefulness of monitoring gamma-H2AX and cell cycle arrest in HepG2 cells for estimating genotoxicity using a high-content analysis system. J Biomol Screen 19(9), 1246-54.

Audebert, M., Dolo, L., Perdu, E., Cravedi, J. P., and Zalko, D. (2011). Use of the gamma H2AX assay for assessing the genotoxicity of bisphenol A and bisphenol F in human cell lines. Arch Toxicol 85(11), 1463-1473.

Bakal, C., Aach, J., Church, G., and Perrimon, N. (2007). Quantitative morphological signatures define local signaling networks regulating cell morphology. Science 316(5832), 1753-1756.

Barlow, N. J., McIntyre, B. S., and Foster, P. M. D. (2004). Male reproductive tract lesions at 6, 12, and 18 months of age following in utero exposure to di(n-butyl) phthalate. Toxicol Pathol 32(1), 79-90.

Belin, B. J., Lee, T., and Mullins, R. D. (2015). DNA damage induces nuclear actin filament assembly by Formin-2 and Spire-1/2 that promotes efficient DNA repair. Elife 4.

Blasi, T., Hennig, H., Summers, H. D., Theis, F. J., Cerveira, J., Patterson, J. O., Davies, D., Filby, A., Carpenter, A. E., and Rees, P. (2016). Label-free cell cycle analysis for high-throughput imaging flow cytometry. Nat Commun 7.

Breiman, L. (2001). Random forests. Mach Learn 45(1), 5-32.

Buchser, W., Collins, M., Garyantes, T., Guha, R., Haney, S., Lemmon, V., Li, Z., and Trask, O. J. (2004). Assay Development Guidelines for Image-Based High Content Screening, High Content Analysis and High Content Imaging. In Assay Guidance Manual (G. S. Sittampalam, N. P. Coussens, K. Brimacombe, A. Grossman, M. Arkin, D. Auld, C. Austin, J. Baell, B. Bejcek, T. D. Y. Chung, et al., Eds.), Bethesda (MD).

Calafat, A. M., Ye, X. Y., Wong, L. Y., Reidy, J. A., and Needham, L. L. (2008). Exposure of the US population to bisphenol A and 4-tertiary-octylphenol: 2003-2004. Environ Health Persp 116(1), 39-44.

Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). CellProfiler: image analysis software for identifying and quantifying cell phenotypes. Genome Biology 7(10).

Chang, C. Y., Leu, J. D., and Lee, Y. J. (2015). The Actin Depolymerizing Factor (ADF)/Cofilin Signaling Pathway and DNA Damage Responses in Cancer. Int J Mol Sci 16(2), 4095-4120.

Chen, B., Co, C., and Ho, C. C. (2015). Cell shape dependent regulation of nuclear morphology. Biomaterials 67, 129-136.

Chen, Y. C., Shu, L., Qiu, Z. Q., Lee, D. Y., Settle, S. J., Hee, S. Q., Telesca, D., Yang, X., and Allard, P. (2016a). Exposure to the BPA-Substitute Bisphenol S Causes Unique Alterations of Germline Function. Plos Genet 12(7).

Chen, Z. J., Zhang, K. S., Ge, L. C., Liu, H., Chen, L. K., Du, J., and Wang, H. S. (2016b). Signals involved in the effects of bisphenol A (BPA) on proliferation and motility of Leydig cells: a comparative proteomic analysis. Toxicol Res-Uk 5(6), 1573-1584.

Cheng, C. Y., and Mruk, D. D. (2002). Cell junction dynamics in the testis: Sertoli-germ cell interactions and male contraceptive development. Physiol Rev 82(4), 825-874.

Chong, Y. T., Koh, J. L. Y., Friesen, H., Duffy, S. K., Cox, M. J., Moses, A., Moffat, J., Boone, C., and Andrews, B. J. (2015). Yeast Proteome Dynamics from Single Cell Imaging and Automated Analysis (vol 161, pg 1413, 2015). Cell 162(1), 221-221.

Conrad, C., and Gerlich, D. W. (2010). Automated microscopy for high-content RNAi screening. Journal of Cell Biology 188(4), 453-461.

De Rooij, D. G., and Russell, L. D. (2000). All you wanted to know about spermatogonia but were afraid to ask. Journal of Andrology 21(6), 776-798.

Driffield, M., Harmer, N., Bradley, E., Fernandes, A. R., Rose, M., Mortimer, D., and Dicks, P. (2008). Determination of brominated flame retardants in food by LC-MS/MS: diastereoisomer-specific hexabromocyclododecane and tetrabromobisphenol A. Food Addit Contam A 25(7), 895-903.

Eidet, J. R., Pasovic, L., Maria, R., Jackson, C. J., and Utheim, T. P. (2014). Objective assessment of changes in nuclear morphology and cell distribution following induction of apoptosis. Diagn Pathol 9.

Elmore, S. A., Ryan, A. M., Wood, C. E., Crabbs, T. A., and Sills, R. C. (2014). FutureTox II: Contemporary Concepts in Toxicology: “Pathways to Prediction: In Vitro and In Silico Models for Predictive Toxicology”. Toxicol Pathol 42(5), 940-942.

EU. (2016). amending Annex XVII to Regulation (EC) No 1907/2006 of the European Parliament and of the Council concerning the Registration, Evaluation, Authorisation and Restriction of Chemicals (REACH) as regards bisphenol A. In (Vol. 2016/2235.

Faridha, A., Faisal, K., and Akbarsha, M. A. (2007). Aflatoxin treatment brings about generation of multinucleate giant spermatids (symplasts) through opening of cytoplasmic bridges: Light and transmission electron microscopic study in Swiss mouse. Reproductive Toxicology 24(3-4), 403-408.

FDA. (2013). Indirect Food Additives: Adhesives and Components of Coatings. In (Vol. 21 CFR 175, pp. 41840 -41843.

FDA. (2012). Indirect Food Additives: Polymers. In (Vol. 21 CFR 177, pp. 41899 -41902.

Fu, S. B., Yang, Y., Tirtha, D., Yen, Y., Zhou, B. S., Zhou, M. M., Ohlmeyer, M., Ko, E. C., Cagan, R., Rosenstein, B. S., et al. (2012). gamma-H2AX Kinetics as a Novel Approach to High Content Screening for Small Molecule Radiosensitizers. Plos One 7(6).

Fuchs, F., Pau, G., Kranz, D., Sklyar, O., Budjan, C., Steinbrink, S., Horn, T., Pedal, A., Huber, W., and Boutros, M. (2010). Clustering phenotype populations by genome-wide RNAi and multiparametric imaging. Mol Syst Biol 6, 370.

Fuller, J. A., Berlinicke, C. A., Inglese, J., and Zack, D. J. (2016). Use of a Machine Learning-Based High Content Analysis Approach to Identify Photoreceptor Neurite Promoting Molecules. Adv Exp Med Biol 854, 597-603.

Gallegos-Avila, G., Ancer-Rodriguez, J., Niderhauser-Garcia, A., Ortega-Martinez, M., and Jaramillo-Rangel, G. (2010). Multinucleation of spermatozoa and spermatids in infertile men chronically exposed to carbofuran. Reprod Toxicol 29(4), 458-60.

Garcia-Canton, C., Anadon, A., and Meredith, C. (2013). Assessment of the in vitro γH2AX assay by High Content Screening as a novel genotoxicity test. Mutation Research/Genetic Toxicology and Environmental Mutagenesis 757(2), 158-166.

Harris, S., Hermsen, S. A. B., Yu, X. Z., Hong, S. W., and Faustman, E. M. (2015). Comparison of toxicogenomic responses to phthalate ester exposure in an organotypic testis co-culture model and responses observed in vivo. Reproductive Toxicology 58, 149-159.

Harris, S., Shubin, S. P., Wegner, S., Van Ness, K., Green, F., Hong, S. W., and Faustman, E. M. (2016a). The presence of macrophages and inflammatory responses in an in vitro testicular co-culture model of male reproductive development enhance relevance to in vivo conditions. Toxicol in Vitro 36, 210-215.

Harris, S., Wegner, S., Hong, S. W., and Faustman, E. M. (2016b). Phthalate metabolism and kinetics in an in vitro model of testis development. Toxicol in Vitro 32, 123-131.

Haywood, M., Spaliviero, J., Jimemez, M., King, N. J. C., Handelsman, D. J., and Allan, C. M. (2003). Sertoli and germ cell development in hypogonadal (hpg) mice expressing transgenic follicle-stimulating hormone alone or in combination with testosterone. Endocrinology 144(2), 509-517.

Heynen-Genel, S., Pache, L., Chanda, S. K., and Rosen, J. (2012). Functional genomic and high-content screening for target discovery and deconvolution. Expert Opin Drug Discov 7(10), 955-68.

Hofmann, M. C., Braydich-Stolle, L., Dettin, L., Johnson, E., and Dym, M. (2005). Immortalization of mouse germ line stem cells. Stem cells (Dayton, Ohio) 23(2), 200-10.

Ibrahim, M. A. A., Elbakry, R. H., and Bayomy, N. A. (2016). Effect of bisphenol A on morphology, apoptosis and proliferation in the resting mammary gland of the adult albino rat. Int J Exp Pathol 97(1), 27-36.

Ikeguchi, M., Sakatani, T., Endo, K., Makino, M., and Kaibara, N. (1999). Computerized nuclear morphometry is a useful technique for evaluating the high metastatic potential of colorectal adenocarcinoma. Cancer 86(10), 1944-1951.

Jones, T. R., Carpenter, A. E., Lamprecht, M. R., Moffat, J., Silver, S. J., Grenier, J. K., Castoreno, A. B., Eggert, U. S., Root, D. E., Golland, P., et al. (2009). Scoring diverse cellular morphologies in image-based screens with iterative feedback and machine learning. P Natl Acad Sci USA 106(6), 1826-1831.

Jones, T. R., Kang, I. H., Wheeler, D. B., Lindquist, R. A., Papallo, A., Sabatini, D. M., Golland, P., and Carpenter, A. E. (2008). CellProfiler Analyst: data exploration and analysis software for complex image-based screens. Bmc Bioinformatics 9.

Kang, J. H., Kondo, F., and Katayama, Y. (2006). Human exposure to bisphenol A. Toxicology 226(2-3), 79-89.

Kierszenbaum, A. L., and Tres, L. L. (2004). The acrosome-acroplaxome-manchette complex and the shaping of the spermatid head. Arch Histol Cytol 67(4), 271-84.

Kitamura, S., Suzuki, T., Sanoh, S., Kohta, R., Jinno, N., Sugihara, K., Yoshihara, S., Fujimoto, N., Watanabe, H., and Ohta, S. (2005). Comparative study of the endocrine-disrupting activity of bisphenol A and 19 related compounds. Toxicological Sciences 84(2), 249-259.

Krewski, D., Acosta, D., Andersen, M., Anderson, H., Bailar, J. C., Boekelheide, K., Brent, R., Charnley, G., Cheung, V. G., Green, S., et al. (2010). Toxicity Testing in the 21st Century: A Vision and a Strategy. J Toxicol Env Heal B 13(2-4), 51-138.

Lakind, J. S., and Naiman, D. Q. (2011). Daily intake of bisphenol A and potential sources of exposure: 2005-2006 National Health and Nutrition Examination Survey. J Expo Sci Env Epid 21(3), 272-279.

Leonard, A. P., Cameron, R. B., Speiser, J. L., Wolf, B. J., Peterson, Y. K., Schnellmann, R. G., Beeson, C. C., and Rohrer, B. (2015). Quantitative analysis of mitochondrial morphology and membrane potential in living cells using high-content imaging, machine learning, and morphological binning. Bba-Mol Cell Res 1853(2), 348-360.

Liang, S., Yin, L., Shengyang Yu, K., Hofmann, M. C., and Yu, X. (2017a). High-Content Analysis Provides Mechanistic Insights into the Testicular Toxicity of Bisphenol A and Selected Analogs in Mouse Spermatogonial Cells. Toxicological sciences : an official journal of the Society of Toxicology 155(1), 43-60.

Liang, S., Yin, L., Shengyang Yu, K., Hofmann, M. C., and Yu, X. (2017b). High-Content Analysis Provides Mechanistic Insights into the Testicular Toxicity of Bisphenol A and Selected Analogs in Mouse Spermatogonial Cells. Toxicol Sci doi: 10.1093/toxsci/kfw178.

Liao, C. Y., and Kannan, K. (2013). Concentrations and Profiles of Bisphenol A and

Other Bisphenol Analogs in Foodstuffs from the United States and Their Implications for Human Exposure. J Agr Food Chem 61(19), 4655-4662.

Liao, C. Y., Liu, F., Guo, Y., Moon, H. B., Nakata, H., Wu, Q., and Kannan, K. (2012). Occurrence of Eight Bisphenol Analogs in Indoor Dust from the United States and Several Asian Countries: Implications for Human Exposure. Environ Sci Technol 46(16), 9138-9145.

Liu, C., Duan, W., Li, R., Xu, S., Zhang, L., Chen, C., He, M., Lu, Y., Wu, H., Pi, H., et al. (2013). Exposure to bisphenol A disrupts meiotic progression during spermatogenesis in adult rats through estrogen-like activity. Cell Death Dis 4, e676.

Liu, C., Duan, W. X., Zhang, L., Xu, S. C., Li, R. Y., Chen, C. H., He, M. D., Lu, Y. H., Wu, H. J., Yu, Z. P., et al. (2014). Bisphenol A exposure at an environmentally relevant dose induces meiotic abnormalities in adult male rats. Cell Tissue Res 355(1), 223-232.

Loo, L. H., Wu, L. F., and Altschuler, S. J. (2007). Image-based multivariate profiling of drug responses from single cells. Nat Methods 4(5), 445-453.

Lui, W. Y., Mruk, D., Lee, W. M., and Cheng, C. Y. (2003). Sertoli cell tight junction dynamics: Their regulation during spermatogenesis. Biology of reproduction 68(4), 1087-1097.

Lyman, S. K., Crawley, S. C., Gong, R., Adamkewicz, J. I., McGrath, G., Chew, J. Y., Choi, J., Holst, C. R., Goon, L. H., Detmer, S. A., et al. (2011). High-Content, High-Throughput Analysis of Cell Cycle Perturbations Induced by the HSP90 Inhibitor XL888. Plos One 6(3).

Martin, H. L., Adams, M., Higgins, J., Bond, J., Morrison, E. E., Bell, S. M., Warriner, S., Nelson, A., and Tomlinson, D. C. (2014). High-Content, High-Throughput Screening for the

Identification of Cytotoxic Compounds Based on Cell Morphology and Cell Proliferation Markers. Plos One 9(2).

Martin, M. T., Knudsen, T. B., Reif, D. M., Houck, K. A., Judson, R. S., Kavlock, R. J., and Dix, D. J. (2011). Predictive Model of Rat Reproductive Toxicity from ToxCast High Throughput Screening. Biol Reprod 85(2), 327-339.

Mata, G., Radojevic, M., Smal, I., Morales, M., Meijering, E., and Rubio, J. (2016). Automatic Detection of Neurons in High-Content Microscope Images Using Machine Learning Approaches. I S Biomed Imaging, 330-333.

Mather, J. P. (1980). Establishment and characterization of two distinct mouse testicular epithelial cell lines. Biology of reproduction 23(1), 243-52.

Mattiazzi Usaj, M., Styles, E. B., Verster, A. J., Friesen, H., Boone, C., and Andrews, B. J. (2016). High-Content Screening for Quantitative Cell Biology. Trends Cell Biol 26(8), 598-611.

Merrick, B. A., Paules, R. S., and Tice, R. R. (2015). Intersection of toxicogenomics and high throughput screening in the Tox21 program: an NIEHS perspective. Int J Biotechnol 14(1), 7-27.

Molina-Molina, J. M., Amaya, E., Grimaldi, M., Saenz, J. M., Real, M., Fernandez, M. F., Balaguer, P., and Olea, N. (2013). In vitro study on the agonistic and antagonistic activities of bisphenol-S and other bisphenol-A congeners and derivatives via nuclear receptors. Toxicol Appl Pharm 272(1), 127-136.

Mylchreest, E., Sar, M., Wallace, D. G., and Foster, P. M. D. (2002). Fetal testosterone insufficiency and abnormal proliferation of Leydig cells and gonocytes in rats exposed to di(n-butyl) phthalate. Reproductive Toxicology 16(1), 19-28.

Neumann, B., Held, M., Liebel, U., Erfle, H., Rogers, P., Pepperkok, R., and Ellenberg, J. (2006). High-throughput RNAi screening by time-lapse imaging of live human cells. Nat Methods 3(5), 385-390.

Neumann, B., Walter, T., Heriche, J. K., Bulkescher, J., Erfle, H., Conrad, C., Rogers, P., Poser, I., Held, M., Liebel, U., et al. (2010). Phenotypic profiling of the human genome by time-lapse microscopy reveals cell division genes. Nature 464(7289), 721-727.

Niedenberger, B. A., Chappell, V. K., Kaye, E. P., Renegar, R. H., and Geyer, C. B. (2013). Nuclear localization of the actin regulatory protein Palladin in sertoli cells. Mol Reprod Dev 80(5), 403-13.

Nurse, P. (1990). Universal Control Mechanism Regulating Onset of M-Phase. Nature 344(6266), 503-508.

O'Brien, P. J., Irwin, W., Diaz, D., Howard-Cofield, E., Krejsa, C. M., Slaughter, M. R., Gao, B., Kaludercic, N., Angeline, A., Bernardi, P., et al. (2006). High concordance of drug-induced human hepatotoxicity with in vitro cytotoxicity measured in a novel cell-based model using high content screening. Arch Toxicol 80(9), 580-604.

Paul Friedman, K., Watt, E. D., Hornung, M. W., Hedge, J. M., Judson, R. S., Crofton, K. M., Houck, K. A., and Simmons, S. O. (2016). Tiered High-Throughput Screening Approach to Identify Thyroperoxidase Inhibitors Within the ToxCast Phase I and II Chemical Libraries. Toxicological Sciences 151(1), 160-80.

Payne, A. H. (1990). Hormonal-Regulation of Cytochrome-P450 Enzymes, Cholesterol Side-Chain Cleavage and 17-Alpha-Hydroxylase C17-20 Lyase in Leydig-Cells. Biology of reproduction 42(3), 399-404.

Pereira, E. F., Aracava, Y., DeTolla, L. J., Jr., Beecham, E. J., Basinger, G. W., Jr., Wakayama, E. J., and Albuquerque, E. X. (2014). Animal models that best reproduce the clinical manifestations of human intoxication with organophosphorus compounds. J Pharmacol Exp Ther 350(2), 313-21.

Peretz, J., Vrooman, L., Ricke, W. A., Hunt, P. A., Ehrlich, S., Hauser, R., Padmanabhan, V., Taylor, H. S., Swan, S. H., VandeVoort, C. A., et al. (2014). Bisphenol A and Reproductive Health: Update of Experimental and Human Evidence, 2007-2013. Environ Health Persp 122(8), 775-786.

Pfeifer, D., Chung, Y. M., and Hu, M. C. T. (2015). Effects of Low-Dose Bisphenol A on DNA Damage and Proliferation of Breast Cells: The Role of c-Myc. Environ Health Persp 123(12), 1271-1279.

Repetto, G., del Peso, A., and Zurita, J. L. (2008). Neutral red uptake assay for the estimation of cell viability/cytotoxicity. Nat Protoc 3(7), 1125-1131.

Richter, C. A., Birnbaum, L. S., Farabollini, F., Newbold, R. R., Rubin, B. S., Talsness, C. E., Vandenbergh, J. G., Walser-Kuntz, D. R., and vom Saal, F. S. (2007). In vivo effects of bisphenol A in laboratory rodent studies. Reprod Toxicol 24(2), 199-224.

Rochester, J. R. (2013). Bisphenol A and human health: A review of the literature. Reproductive Toxicology 42, 132-155.

Schmitz, M. H., Held, M., Janssens, V., Hutchins, J. R., Hudecz, O., Ivanova, E., Goris, J., Trinkle-Mulcahy, L., Lamond, A. I., Poser, I., et al. (2010). Live-cell imaging RNAi screen identifies PP2A-B55alpha and importin-betal as key mitotic exit regulators in human cells. Nat Cell Biol 12(9), 886-93.

Setchell, B. P. (2008). Blood-Testis Barrier, Junctional and Transport Proteins and Spermatogenesis. Adv Exp Med Biol 636, 212-233.

Shukla, S. J., Huang, R., Austin, C. P., and Xia, M. (2010). The future of toxicity testing: a focus on in vitro methods using a quantitative high-throughput screening platform. Drug Discov Today 15(23-24), 997-1007.

Singh, S., Carpenter, A. E., and Genovesio, A. (2014). Increasing the Content of High-Content Screening: An Overview. Journal of Biomolecular Screening 19(5), 640-650.

Sommer, C., and Gerlich, D. W. (2013). Machine learning in cell biology—teaching computers to recognize phenotypes. Journal of Cell Science 126(24), 5529-5539.

Stossi, F., Bolt, M. J., Ashcroft, F. J., Lamerdin, J. E., Melnick, J. S., Powell, R. T., Dandekar, R. D., Mancini, M. G., Walker, C. L., Westwick, J. K., et al. (2014). Defining Estrogenic Mechanisms of Bisphenol A Analogs through High Throughput Microscopy-Based Contextual Assays. Chem Biol 21(6), 743-753.

Su, L., Agati, J. M., and Parsons, S. J. (2003). p190RhoGAP is cell cycle regulated and affects cytokinesis. Journal of Cell Biology 163(3), 571-582.

Sun, X., Kovacs, T., Hu, Y. J., and Yang, W. X. (2011). The role of actin and myosin during spermatogenesis. Mol Biol Rep 38(6), 3993-4001.

Sutherland., V., McIntyre., B., Pelch., K., Waidyanatha., S., Conley., J. M., Gray., L. E., and Foster, P. M. A Comparison of In Vivo Reproductive and Developmental Toxicity (DART) Endpoints for Bisphenol AF and Bisphenol A2017, Baltimore, Md.

Takao, T., Nanamiya, W., Nagano, I., Asaba, K., Kawabata, K., and Hashimoto, K. (1999). Exposure with the environmental estrogen bisphenol A disrupts the male reproductive tract in young mice. Life Sci 65(22), 2351-2357.

Tarca, A. L., Carey, V. J., Chen, X. W., Romero, R., and Draghici, S. (2007). Machine learning and its applications to biology. Plos Comput Biol 3(6), 953-963.

Vandenberg, L. N., Hauser, R., Marcus, M., Olea, N., and Welshons, W. V. (2007). Human exposure to bisphenol A (BPA). Reproductive Toxicology 24(2), 139-177.

Vidal, J. D., and Whitney, K. M. (2014). Morphologic manifestations of testicular and epididymal toxicity. Spermatogenesis 4(2), e979099.

vom Saal, F. S., Akingbemi, B. T., Belcher, S. M., Birnbaum, L. S., Crain, D. A., Eriksen, M., Farabollini, F., Guillette, L. J., Hauser, R., Heindel, J. J., et al. (2007). Chapel Hill bisphenol A expert panel consensus statement: Integration of mechanisms, effects in animals and potential to impact human health at current levels of exposure. Reproductive Toxicology 24(2), 131-138.

Wang, L., Wang, M., Wang, S. Y., Qi, T. Y., Guo, L. J., Li, J. J., Qi, W. J., Ampah, K. K., Ba, X. Q., and Zeng, X. L. (2013). Actin Polymerization Negatively Regulates p53 Function by Impairing Its Nuclear Import in Response to DNA Damage. Plos One 8(4).

Wong, E. W. P., Mruk, D. D., and Cheng, C. Y. (2008). Biology and regulation of ectoplasmic specialization, an atypical adherens junction type, in the testis. Bba-Biomembranes 1778(3), 692-708.

Yin, L., Wei, H., Liang, S., and Yu, X. (2017). An animal-free in vitro three-dimensional testicular cell co-culture model for evaluating male reproductive toxicants. Toxicological sciences : an official journal of the Society of Toxicology, 43-60.

Yu, X. Z., Hong, S., Moreira, E. G., and Faustman, E. M. (2009). Improving in vitro Sertoli cell/gonocyte co-culture model for assessing male reproductive toxicity: Lessons learned from comparisons of cytotoxicity versus genomic responses to phthalates. Toxicol Appl Pharm 239(3), 325-336.

Yu, X. Z., Sidhu, J. S., Hong, S., and Faustman, E. M. (2005). Essential role of extracellular matrix (ECM) overlay in establishing the functional integrity of primary neonatal rat sertoli cell/gonocyte co-cultures: An improved In vitro model for assessment of male reproductive toxicity. Toxicological Sciences 84(2), 378-393.

Zanella, F., Lorens, J. B., and Link, W. (2010). High content screening: seeing is believing. Trends Biotechnol 28(5), 237-245.

Zuchero, J. B., Belin, B., and Mullins, R. D. (2012). Actin binding to WH2 domains regulates nuclear import of the multifunctional actin regulator JMY. Mol Biol Cell 23(5), 853-863.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A composition comprising cells and a protein matrix,
wherein the cells comprise immortalized spermatogonial cells, immortalized Sertoli cells, and immortalized Leydig cells,
wherein the spermatogonial cells are present at 70-90%, the Sertoli cells are present at 10-20%, the Leydig cells are present at 1-10%, and the spermatogonial cells, Sertoli cells, and Leydig cells add up to 100% of the cells in the composition,
wherein the protein matrix comprises a protein mixture representing an extracellular matrix.

2. The composition of claim 1 wherein the Sertoli cells comprise TM3 cells.

3. The composition of claim 1 wherein the Leydig cells comprise TM4 cells.

4. The composition of claim 1 wherein the spermatogonial cells comprise C18-4 cells.

5. The composition of claim 1 wherein the protein mixture represents an extracellular microenvironment comprising extracellular matrix proteins.

6. The composition of claim 1 wherein the composition comprises a three-dimensional F-actin cytoskeleton.

7. A method for producing a cell culture, the method comprising:
combining cells and a protein matrix in a container to result in a cell culture,
wherein the cells are immortalized spermatogonial cells, immortalized Sertoli cells, and immortalized Leydig cells,
wherein the spermatogonial cells are present at 70-90%, the Sertoli cells are present at 10-20%, the Leydig cells are present at 1-10%, and the spermatogonial cells, Sertoli cells, and Leydig cells add up to 100%,
wherein the protein matrix comprises a protein mixture representing an extracellular microenvironment.
incubating the cell culture under conditions suitable for maintaining viability of the cells.

8. The method of claim 7 wherein the Sertoli cells comprise TM3 cells.

9. The method of claim 7 wherein the Leydig cells comprise TM4 cells.

10. The method of claim 7 wherein the spermatogonial cells comprise C18-4 cells.

11. The method of claim 7 wherein at least 10 micrograms/ml (μg/ml) to no greater than 200 μg/ml protein matrix is combined.

12. The method of claim 7 wherein the incubating comprises incubation until a three-dimensional F-actin cytoskeleton is formed by the cell culture.

13. A method comprising:
providing the composition of claim 1;
contacting cells in the composition with a compound to form a mixture;
incubating the mixture under conditions suitable for maintaining viability of the cells in the absence of the compound; and
determining the status of cells.

14. The method of claim 13 wherein the status comprises cell viability.

15. The method of claim 14 wherein the compound reduces the cell viability of cells.

16. The method of claim 14 wherein the determining comprises measuring neutral red uptake capacity of the cells.

17. The method of claim 14 further comprising determining whether the compound affects cell viability of the spermatogonial cells, the Sertoli cells, the Leydig cells, or a combination thereof.

18. The method of claim 14 wherein the cell viability of cells is not reduced by the compound.

19. The method of claim 14 wherein the determining comprises calculating an inhibitory concentration (IC) of the compound.

20. The method of claim 19 wherein the IC calculated is IC₅₀.

21. A method for identifying a toxic compound, the method comprising contacting the composition of claim 1 with a compound and analyzing viability of the cells, wherein a reduction of viability indicates the compound is a toxic compound.
